SEQUENCE LISTING (76 sequences)

SEQ ID NO: 1 - DNA, 1122 bp, Bacillus subtilis
atgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaa

SEQ ID NO: 2 - protein, 373 aa, Bacillus subtilis
MSQHVETKLAQIGNRSDEVTGTVSAPIYLSTAYRHRGIGESTGFDYVRTKNPTRQLVEDAIANLENGARGLAFSSGMAAIQTIMALFKSGDELIVSSDLYGGTYRLFENEWKKYGLTFHYDDFSDEDCLRSKITPNTKAVFVETPTNPLMQEADIEHIARITKEHGLLLIVDNTFYTPVLQRPLELGADIVIHSATKYLGGHNDLLAGLVVVKDERLGEEMFQHQNAIGAVLPPFDSWLLMRGMKTLSLRMRQHQANAQELAAFLEEQEEISDVLYPGKGGMLSFRLQKEEWVNPFLKALKTICFAESLGGVESFITYPATQTHMDIPEEIRIANGVCNRLLRFSVGIEHAEDLKEDLKQALCQVKEGAVSFE

SEQ ID NO: 3 - DNA, 195 bp, Artificial Sequence
  Description of Artificial Sequence: Synthetic promoter sequence
gctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgtt

SEQ ID NO: 4 - DNA, 156 bp, Artificial Sequence
  Description of Artificial Sequence: Synthetic promoter sequence
ccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttt

SEQ ID NO: 5 - DNA, 22 bp, Artificial Sequence
  Description of Artificial Sequence: Synthetic oligonucleotide
tctagaaggaggagaaaacatg

SEQ ID NO: 6 - DNA, 24 bp, Artificial Sequence
  Description of Artificial Sequence: Synthetic oligonucleotide
tctagaccaggaggacatacagtg

SEQ ID NO: 7 - DNA, 8970 bp, Artificial Sequence
  Description of Artificial Sequence: Synthetic vector sequence
ggatcggcggccagggccctcatgagatatcgagtggatttgtgcaaaactttcaggtgtgcgatgcatgagaatctgcccaataaaattaagtttgcctcggcgatagaggtctccgtcaataacatcgtcatgaaccaaaagggaaaaatgcagtagttctaaagccactgctacctgtaaaacggtgttgagtttgacctcaatgtcatcgtctacaagcgtgttgtatagccccagtagcattcgagggcggattaacttgccacctcgcaaagcttggaaagcagcatctaggcaggtacggaactctggttgatatgtgctgcactgttgagatagcgaagcgcagatgcggtttagttcccgataaatctcatcattgaaatcaagatcaggatgagttgaatgttctgtggtgattgtcatgccattgtccattcgagtatcacacggccagttatctcgcaaaaattcccaatcgttgtatatggcgctttattttgatgaagtacagaaagtgtgaatttgggtccataaaaataatgtgcctacaagaaatttatagtatcccatgagttaatatttttaaaaataaactttatctgactttgtagaaaaaggtgattactatgctgaatatgcaggaaccagataaaatccatccggcagaacctacacttcgtaatatttatgacgttaaaactagtgatcccaaaagtgaattagttgatcgttctggcatgtcggaagaagacattgcgcaaattgggcggctaatgaaatcgttggccagtcttcgcgatgtggaacgtagtattggtgaagcctcggcacgttatatggagctaagtgcccctgatatgcgagctttgcactatttgattgtggcgggcaatgcgggcgaagtggtgactccaggaatgcttggagctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggagggattcatcatgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatagggggatcgatatccgtttaggctgggcggatccgccctcccgcacgctttgcgggagggcggtaccagctcaccttaagctttccccggcatctgtaacaaagacgcttaataggctagaaaaaggtgggcatattgttcgtaatgtgcaccccgtcgaccgcagggctttcgccctcatggtcactgatgccactcgtggagaggcgatgcggacgcttggtaagcatcaggcgcgtcgttttgatgctgctaaacgattaactccacaagagcgtgaagtggttatccgattccttcaggatatggcacaggagttatcccttaataatgcaccatggctcaacacggagtagatgaccatctacgttaattaaagtgtgcagagcggagtggcggtgtttaagccacctgtcgctgggactgtaatgaatgcgcatggccaccacccactgtcctctgtaatgttccgaacgtgagaccattggtcactactgagctgtggcgtgcgggatagtataaatcctgaggaccggcttgggctgccgacgattgctagtgaataatcatcttcgatataggtcacgcggtagtttgcttgattgtcttcactctgaaatggaatacctgggaagctaacctttaatgaagcattggaaactactttagcgctgccttcaataactgaaggcccaaagaaagtgccacacttatttgttacagagattgtgtccgagtcgatcacgccgtaatcagcggtaacgtcatgtgagcactgtaaagagaatggttggggaattgctgcgacttgataccacttgcctttgtagcgttctaggtcaatgctattttcaatttcgggcagcgctaggttttcaggaaccgaacttaggttagatacctgcgaggagccacctgcaagtcgtccgccgtcaaaaatgtcttgggcttgtgccgtggatatcccgaaaagtgaaatggctgcgagtagtgctgtggtgacaagtttgcttgaaatgcgcataaagcaaatcctttcttcatgtttatattaactcaatagttattacttctaaaagtatagtagatagttgtggatgggtgaagaatttcatagaaatcgcactcgattcactaaagacccaagagtaaaatcccaggatttgcttatacttgcgctcatggataatcaacttcgtcccactttgcattatcaagctcaaaacccgcaccctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 8 - DNA, 12383 bp, Artificial Sequence
  Description of Artificial Sequence: Synthetic vector sequence
ggatcggcggccagggccctcatgagatatcgagtcagcgctgtattgcccgtgaagttgatggtgtttccgctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagagatccccagcttgttgatacactaatgcttttatatagggaaaaggtggtgaactactgtggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggggatccgccctcccgcacgctttgcgggagggcttttcttttaccggtaccagctcagattagcttcccggtctgcattaacatcctgtactgctccaaggatctgactggccatgccccacaagaaaaaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 9 - DNA, 11378 bp, Artificial Sequence
  Description of Artificial Sequence: Synthetic vector sequence
ggatcggcggccagggccctcatgagatatcgagtggatttgtgcaaaactttcaggtgtgcgatgcatgagaatctgcccaataaaattaagtttgcctcggcgatagaggtctccgtcaataacatcgtcatgaaccaaaagggaaaaatgcagtagttctaaagccactgctacctgtaaaacggtgttgagtttgacctcaatgtcatcgtctacaagcgtgttgtatagccccagtagcattcgagggcggattaacttgccacctcgcaaagcttggaaagcagcatctaggcaggtacggaactctggttgatatgtgctgcactgttgagatagcgaagcgcagatgcggtttagttcccgataaatctcatcattgaaatcaagatcaggatgagttgaatgttctgtggtgattgtcatgccattgtccattcgagtatcacacggccagttatctcgcaaaaattcccaatcgttgtatatggcgctttattttgatgaagtacagaaagtgtgaatttgggtccataaaaataatgtgcctacaagaaatttatagtatcccatgagttaatatttttaaaaataaactttatctgactttgtagaaaaaggtgattactatgctgaatatgcaggaaccagataaaatccatccggcagaacctacacttcgtaatatttatgacgttaaaactagtgatcccaaaagtgaattagttgatcgttctggcatgtcggaagaagacattgcgcaaattgggcggctaatgaaatcgttggccagtcttcgcgatgtggaacgtagtattggtgaagcctcggcacgttatatggagctaagtgcccctgatatgcgagctttgcactatttgattgtggcgggcaatgcgggcgaagtggtgactccaggaatgcttggagctgcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagagatccccagcttgttgatacactaatgcttttatatagggaaaaggtggtgaactactgtggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggggatccgccctcccgcacgctttgcgggagggcttttcttttaccggtaccagctcaccttaagctttccccggcatctgtaacaaagacgcttaataggctagaaaaaggtgggcatattgttcgtaatgtgcaccccgtcgaccgcagggctttcgccctcatggtcactgatgccactcgtggagaggcgatgcggacgcttggtaagcatcaggcgcgtcgttttgatgctgctaaacgattaactccacaagagcgtgaagtggttatccgattccttcaggatatggcacaggagttatcccttaataatgcaccatggctcaacacggagtagatgaccatctacgttaattaaagtgtgcagagcggagtggcggtgtttaagccacctgtcgctgggactgtaatgaatgcgcatggccaccacccactgtcctctgtaatgttccgaacgtgagaccattggtcactactgagctgtggcgtgcgggatagtataaatcctgaggaccggcttgggctgccgacgattgctagtgaataatcatcttcgatataggtcacgcggtagtttgcttgattgtcttcactctgaaatggaatacctgggaagctaacctttaatgaagcattggaaactactttagcgctgccttcaataactgaaggcccaaagaaagtgccacacttatttgttacagagattgtgtccgagtcgatcacgccgtaatcagcggtaacgtcatgtgagcactgtaaagagaatggttggggaattgctgcgacttgataccacttgcctttgtagcgttctaggtcaatgctattttcaatttcgggcagcgctaggttttcaggaaccgaacttaggttagatacctgcgaggagccacctgcaagtcgtccgccgtcaaaaatgtcttgggcttgtgccgtggatatcccgaaaagtgaaatggctgcgagtagtgctgtggtgacaagtttgcttgaaatgcgcataaagcaaatcctttcttcatgtttatattaactcaatagttattacttctaaaagtatagtagatagttgtggatgggtgaagaatttcatagaaatcgcactcgattcactaaagacccaagagtaaaatcccaggatttgcttatacttgcgctcatggataatcaacttcgtcccactttgcattatcaagctcaaaacccgcaccctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 10 - DNA, 10322 bp, Artificial Sequence
  Description of Artificial Sequence: Synthetic plasmid sequence
ggatcggcggccagggccctcatgagatatcgagtcagcgctgtattgcccgtgaagttgatggtgtttccgctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaaggaggagaaaacatgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 11 - DNA, 10324 bp, Artificial Sequence
  Description of Artificial Sequence: Synthetic plasmid sequence
ggatcggcggccagggccctcatgagatatcgagtcagcgctgtattgcccgtgaagttgatggtgtttccgctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaccaggaggacatacagtgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 12 - DNA, 10470 bp, Artificial Sequence
  Description of Artificial Sequence: Synthetic vector sequence
ggatcggcggccagggccctcatgagatatcgagtcagcgctgtattgcccgtgaagttgatggtgtttccgctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggagaaaacatgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 13 - DNA, 10484 bp, Artificial Sequence
  Description of Artificial Sequence: Synthetic plasmid sequence
ggatcggcggccagggccctcatgagatatcgagtcagcgctgtattgcccgtgaagttgatggtgtttctctagaccaggaggacatacagtggctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgagaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccacgatctagaccaggaggacatacagtgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 14 - DNA, 12531 bp, Artificial Sequence
  Description of Artificial Sequence: Synthetic vector sequence
ggatcggcggccagggccctcatgagatatcgagtcagcgctgtattgcccgtgaagttgatggtgtttccgctgccctgctgggtgggattggaggtgtaatcaatgaaccaaccaggagttccggtgccagtgagatcaaataccacgcggtcaaagccactgtgagagccaatccgaacatcggtgaccatgagctgtgcaggcgcatcaggtcggagagtcttcattgctacatcggcttcgcccaatgcggttgggccggtggaagcttcgttggacaactgtgcgccatccgcagttgcggacatagtttgggttacagaagaagcatcgttggtggtggaattggaggttccacaacccgcaagagtcaacgcgctagcgccgacaatcgctagagtcttcaggcgggcacgatgctttgaatgagaagttggctgcacaatcatgcacacaccgtaaccctgggtcacccccgaaacctaagcaagacgcccaatttcgctcaatcgtgaacgaattgttgtaattcgtcttaaaaacgccaggagacgtgaaaattacagacaccccagacatcagatggaggcggcgatactagggtagaggacatgactcttcgctgttctgacgtcaatgttgaacccctgccgggaacggcaaaaacaggttctgggtttgttctccttgaacatgctggctcgtggagccgtgatgttttagacggcggaacatttgatcctgagttgactgatcaattgaagaggcacctgaaagcttccggaatgggtctgcaattaattaggaagccgggaagggagggtcgaaacgtcgaaaagcataatctttttctcgtttttgctgaggcctcaattattgagcacctggtggtggacgcgccggctgatgttttggatcttgatttaagcgggccgggcaaaaacaatgcgcagcgcatggatgatccgatgctgctgatttgtacgcattcgaagcgcgatgtgtgctgcgcgatcaaggggcgtccgctggcagctgccgtggagccacaatttgggccgctgcatgtgtgggaggcttcgcacaccaagggccaccgttttgcgccatcgatgctgctcatgccgtggaattactcttatggcctacttgatgaggccgaaaccgtgcagcttttccaaggcgcgttggacaacaaactcttcctgccgggcaaccgtggccgaggaaccttagatgctcgtggccaggttgcagaaattgccgtggcggaagctttcggcgaggcggttgctcctgcgagtttgcaggttgaattcgaagatgattctgttttggttactcatcccgatgggcgcacgtgggttgtggagcttgaacgcatcgaggtcgacggcgtggtgtcctcgtgtggtgatcagccgaaaactggaaaagcgtgggtggctaggcaagttacagaactgatcggataaaagcagagttatatctgatgaattgctattagcagtatcgttatcacagcaccaacaaagtagttcagccacaggaaaactttccaactgcgattagcctgttcacaactggcatctgtaatgttccaaaatcgtgcggcattaaatacgtaagttagaatcgcaatcccgatgatccacgccggattaggcaaagtagtgactaacacagcagctagtaaataaagtactactgaaagccgaatggctccacgcgccccaattacagtggcaattgagctgcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagagatccccagcttgttgatacactaatgcttttatatagggaaaaggtggtgaactactgtggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggggatccgccctcccgcacgctttgcgggagggcttttcttttaccggtaccagctcagattagcttcccggtctgcattaacatcctgtactgctccaaggatctgactggccatgccccacaagaaaaaggatcccagtgctatccacatcgctgctgaaggagatgttccagtgatcgttgcaccgattaatgcaggtgaagtgaagtgagtagaagatgttagagcatcgataaaggggcgttctttaaaacgcaatttcggtgctgaataagcaatcactgctagcactgagagtgtcagccataaagacgacatccaggtgccaaatatgaaaagaataactaggaaaggaattgttgagatagccgaggcccataacagtgtgctgtgggaacttttcggtagcacggccccctcgacgccgcctttgcggggattacgcatatcagattcgtaatcaaaaacatcgttgataccatacatggcgatgttatacgggataagaaaaaatacgatgcctagccaaaacagccagtcaatctctcctgcatttaataggtaggccagaccaaaggggtaggcggtattgatccagctaatggggcgagatgacaatagaattagtcttattttttccatcatgactacggcttttctggctcagattgcgtggtggtggatctagtagtgatgcttccattggcgatggtgggtaaggaatggtgtggacgttttttcctgcgtttaaacatatttccaggcaaccatagggcaggaatcagaagtactgcgaagagcggatagaaaagatcctctagggggattaaaccgagccaaatgccaaggtgctgggtatcgccatatccaaagagatcagcccaaaccatgaggttatcaaatatgatagttagggaacatagggtaagggcactgacagcggtgattggtaaaagtttaggtgttccagactgcagctttaagacaaataggaccatggctattgctaaaaaaggaatgcttataaaaatataagtcatggttcaacctcgggagtggtagttggttggaaagtatcgcgctgtggtgtgaggggagactttttaccgggttttttaggcagtggtgctttaagccataatgctgctgccgaggtaaggttgagggtgatgtagcagaggaagaataagaaaaaaagttcttcaatgggcatatggggtgcaaggttaataccggacataaacgctgagtctccgcgataaaaagtgccagtaataatgccaaatatatcccataaaagaaatccaatatatgcagcacctaccgaaagaattgctcgtaacggatggcggaagaacgctagcttccaacggtggtcgcacaaagccatgcacccaatgagaactaggagagtacctagataaataaaggccataaaaatatcgctatcttgctcattttgtgaaatatcgatgatagggatcaaaatttaatgatcgtatgaggtcttttgagatggtgtcgttttaggcggcaatggttcggctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 15 - DNA, 8554 bp, Artificial Sequence
  Description of Artificial Sequence: Synthetic vector sequence
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggagggattcatcatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgctcgatttaaatc

SEQ ID NO: 16 - DNA, 183 bp, Artificial Sequence
  Description of Artificial Sequence: Synthetic promoter sequence
accctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccacga

SEQ ID NO: 17 - DNA, 192 bp, Artificial Sequence
  Description of Artificial Sequence: Synthetic promoter sequence
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 18 - DNA, 83 bp, Artificial Sequence
  Description of Artificial Sequence: Synthetic promoter sequence
tacgttaaatctatcaccgcaagggataaatatctaacaccgtgcgtgttgactattttacctctgcggtgataatggttgca

SEQ ID NO: 19 - DNA, 86 bp, Artificial Sequence
  Description of Artificial Sequence: Synthetic promoter sequence
taaaaaacatacagataaccatctgcggtgataaattatctctggcggtgttgacataaataccactggcggtgatactgagcaca

SEQ ID NO: 20 - DNA, 9462 bp, Artificial Sequence
  Description of Artificial Sequence: Synthetic plasmid sequence
ttgatcagcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggagaaaacatgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaagcctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatccgccctcccgcacgctttgcgggagggcttttcttttcccggtatttatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggataaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 21 - DNA, 6779 bp, Artificial Sequence
  Description of Artificial Sequence: Synthetic plasmid sequence
agaggatccgactgtttcagaagtgatgactcctgaaaatttgggcgcgctgtatgacatgtcggtgtcgttggaaactgtgcgcagccggtggttcgcgttcgatgctctgcattaaaaggggctagttttacacaaaagtggacagcttggtctatcattgccagaagaccggtccttttagggccatagaattctgattacaggagttgatctaccttgtcttttgacccaaacacccagggtttctccactgcatcgattcacgctgggtatgagccagacgactactacggttcgattaacaccccaatctatgcctccaccaccttcgcgcagaacgctccaaacgaactgcgcaaaggctacgagtacacccgtgtgggcaaccccaccatcgtggcattagagcagaccgtcgcagcactcgaaggcgcaaagtatggccgcgcattctcctccggcatggctgcaaccgacatcctgttccgcatcatcctcaagccgggcgatcacatctgtttaagtttagtggatgggccaggtctgaagaaccacccaggccacgaagtcgcagcgaagcagatgaagcgcttcggcggcatgatctccgtccgtttcgcaggcggcgaagaagcagctaagaagttctgtacctccaccaaactgatctgtctggccgagtccctcggtggcgtggaatccctcctggagcacccagcaaccatgacccaccagtcagctgccggctctcagctcgaggttccccgcgacctcgtgcgcatctccattggtattgaagacattgaagacctgctcgcagatgtcgagcaggccctcaataacctttagaaactatttggcggcaagcagcttttcaatataagcaatgcgagcctccaccatgtagccgaagagttcgtcagaagttgagacggactcttcgactgctttacgggtcagtggcgcttccacatctgggttctcatcaagccatggcttaggaaccggagcaaacacatccggcttttcgccctctggacgattgtcaaaggtgtagtcggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactct

SEQ ID NO: 22 - DNA, 6934 bp, Artificial Sequence
  Description of Artificial Sequence: Synthetic plasmid sequence
tcgagaggcctgacgtcgggcccggtaccacgcgtcatatgactagttcggacctagggatcttcaaccctcctctcgccgcttgagactgtgattgggcttacctctttggcttcgaacaccggggttgaggtagtggttacttccatggtttcctcagcggaaacggcttggctatcagcactttcacccgaacagcctgcaagaagtgcgacggctaacagggctgggattgtcctcaacttcacttcgggctccttcttagtaataggttcgtagaaaagtttactagcctagagagtatgcgatttcctgaactcgaagaattgaagaatcgccggaccttgaaatggacccggtttccagaagacgtgcttcctttgtgggttgcggaaagtgattttggcacctgcccgcagttgaaggaagctatggcagatgccgttgagcgcgaggtcttcggatacccaccagatgctactgggttgaatgatgcgttgactggattctacgagcgtcgctatgggtttggcccaaatccggaaagtgttttcgccattccggatgtggttcgtggcctgaagcttgccattgagcatttcactaagcctggttcggcgatcattgtgccgttgcctgcataccctcctttcattgagttgcctaaggtgactggtcgtcaggcgatctacattgatgcctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggagggattcatcatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttggatccgccctcccgcacgctttgcgggagggcggtaccagctcaccgatattgcggcgaagtacgatgcccgcatcatcgtcgatgagatccacgcgccactggtttatgaaggcacccatgtggttgctgctggtgtttctgagaacgctgcaaacacttgcatcaccatcaccgcaacttctaaggcgtggaacactgctggtttgaagtgtgctcagatcttcttcagtaatgaagccgatgtgaaggcctggaagaatttgtcggatattacccgtgacggtgtgtccatccttggattgatcgctgcggagacagtgtacaacgagggcgaagaattccttgatgagtcaattcagattctcaaggacaaccgtgactttgcggctgctgaactggaaaagcttggcgtgaaggtctacgcaccggactccacttatttgatgtggttggacttcgctggcaccaagatcgaagaggcgccttctaaaattcttcgtgaggagggtaaggtcatgctgaatgatggcgcagcttttggtggtttcaccacctgcgctcgtcttaattttgcgtgttccagagagacccttgaggaggggctgcgccgtatcgccagcgtgttgtaaataatgagtaaaaagtctgtcctgattacttctttgatgctgttttccatgttcttcggagctggaaacctcatcttcccgccgatgcttggattgtcggcaggaaccaactatctaccagctatcttaggatttctagcaacgagtgttctgctcccggtgctggcgattatcgcggtggtgttgtcgggagaaaatgtcaaggacatggcttctcgtggcggtaagatctttggcctggtgtttcctattgctgcctatttgtctatcggcgcgttttacgcgctgccgaggactggggcggtgagctattcgacggcggttggcgtcgataatgcgctttattcgggcttgtttaactttgtgttttttgcggtggcactggcgttgtcgtggaatccgaatggcattgcagacaagttgggtaagtggctcacgccagcgttgctcacgttgattgtggtgctggtggtgttgtcggtagctaagttgatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaacaccccgagtccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgcatgcatccatttaaatggaagaaataaaagaaaatgccaataggatattggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 23 - DNA, 1851 bp, Artificial Sequence
  Description of Artificial Sequence: Synthetic plasmid sequence
gttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagaataaatttataccacacagtctattgcaatagaccaagctgttcagtagggtgcatgggagaagaatttcctaataaaaactcttaaggacctccaaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagctttaaatagactcaccccagtgcttaaagcgctgggtttttctttttcagactcgtgagaatgcaaactagactagacagagctgtccatatacactggacgaagttttagtcttgtccacccagaacaggcggttattttcatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagcc

SEQ ID NO: 24 - DNA, 8102 bp, Artificial Sequence
  Description of Artificial Sequence: Synthetic plasmid sequence
ttgatcagcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggagaaaacatgtcacagcacgttgaaacgaaattagctcaaattgggaaccgtagcgatgaagtcacgggaacagtgagtgctcctatctatttatcaacagcataccgccacagagggatcggagaatctaccggatttgattatgtccgcacaaaaaatccgacacgccagcttgttgaggacgcgatcgctaacttagaaaacggcgcgagagggcttgcctttagttcgggaatggctgctatccaaacgattatggcgctgtttaaaagcggagatgaactgatcgtttcatcggacctatatggcggcacgtaccgtttatttgaaaatgaatggaaaaaatacggattgacttttcattatgatgatttcagcgatgaggactgtttacgctctaagattacgccgaatacaaaagcggtgtttgtggaaacgccgacaaaccccctcatgcaggaggcggacattgaacatattgcccggattacaaaggagcacggtcttctgctgatcgtagataatacattttatacaccggtcttgcagcggccgcttgagctgggagctgacattgtcattcacagcgcaaccaagtatttaggcgggcataacgatctgcttgctggacttgtcgtggtgaaggatgagcggctcggagaggaaatgtttcagcatcaaaatgcaatcggcgccgtcctgccgccatttgattcgtggcttctgatgagaggaatgaagacgctgagcctcagaatgcgccagcatcaggcaaacgcgcaggagcttgcggcgtttttagaagagcaggaagaaatttcggatgtgctgtatcccggaaaaggcggcatgctgtccttccgtctgcaaaaagaagaatgggtcaatccgtttttaaaagcactgaagaccatttgttttgcagaaaacctcggcggggtggaaagctttattacataccctgcgacccagacgcacatggatattcctgaagagatccgcatcgcaaacggggtgtgcaatcggttgctgcgcttttctgtcggtattgaacatgcggaagatttaaaagaggatctaaaacaggcattatgtcaggtcaaagagggagctgtttcatttgagtaaacacaattggacgctggaaacccagctcgtgcacaatccatttaaaacagacggcggaaccggggcagtcagtgtaccgattcagcacgcctcaggatccgccctcccgcacgctttgcgggagggcttttcttttcccggtatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 25 - DNA, 9627 bp, Artificial Sequence
  Description of Artificial Sequence: Synthetic vector sequence
ggatcggcggccagggcccagatttcttcttgttctctgttgtgatgctagtagacatcaaattcacttccgttaaaggcgtaaaggttagttctaagatttgcggctggctagttgctcaacttgtttaagtaataaattttgagccttgggatctacctgggggcttagtgttttgatcatcgaacggcagatatttaagtggtcggagatggcattcaaggagtgattctttgctccacactgtatgagaagatgttgaattcgctctgcctgagaggtgctcagatctgcggagccgaagttgtcttgaatatcagtccatttagcagtatctcgagcgaaggcgataattgtagtttcttttccttctcgaaggtcagaaaaggcatctttgccgtgttcggctgcgtcaccaaaagtagataagtaatcgtcctgcaattgataagcgatgcccaagtttgtgcctatctcacctatctttgtttcaatctcctgaggtagttccgcgagaattgccgctgctctcattggaagttcaaaagtgtatgtggctgtttttagccgactcatttctagagcaatgtccatattggggctgatggctttgctgcttaatcccacatcaagaaactcacccacaatagtgtcatttatcgtgtggttgagtaaatctaaaagtcgaacccgttggtgatgtggaaggtcaaggcgcgcgaagatttgatgggtggcagcaagaaaaagatttcccataagcagtccattagattgtgcccaatctagatgctctggatcgcgctggatttgtgcaaaactttcaggtgtgcgatgcatgagaatctgcccaataaaattaagtttgcctcggcgatagaggtctccgtcaataacatcgtcatgaaccaaaagggaaaaatgcagtagttctaaagccactgctacctgtaaaacggtgttgagtttgacctcaatgtcatcgtctacaagcgtgttgtatagccccagtagcattcgagggcggattaacttgccacctcgcaaagcttggaaagcagcatctaggcaggtacggaactctggttgatatgtgctgcactgttgagatagcgaagcgcagatgcggtttagttcccgataaatctcatcattgaaatcaagatcaggatgagttgaatgttctgtggtgattgtcatgccattgtccattcgagtatcacacggccagttatcacgcatgtattctcctccttactagaacaggcggggttgcccccgcctgtaattaaattattacacaccctgtagggaaagtcaatacctttttgtaaaatttttacacagcgtggatctcttctagggacacctctttgtacccctcaagggagaaatattggcggtactgagcacagttttggttggtggacagtgaaccatagctgtcgtcaatagcctcgagttatggcagttggttaaaaggaaacaaaaagaccgttttcacacaaaacggtctttttcgatttctttttacagtcacagccacttttgcaaaaaccggacagcttcatgccttataactgctgtttcgaagcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggagggattcatcatgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatagggggatcgatatccgtttaggctgggcggatccgccctcccgcacgctttgcgggagggcggtaccagctcaccttaagctttccccggcatctgtaacaaagacgcttaataggctagaaaaaggtgggcatattgttcgtaatgtgcaccccgtcgaccgcagggctttcgccctcatggtcactgatgccactcgtggagaggcgatgcggacgcttggtaagcatcaggcgcgtcgttttgatgctgctaaacgattaactccacaagagcgtgaagtggttatccgattccttcaggatatggcacaggagttatcccttaataatgcaccatggctcaacacggagtagatgaccatctacgttaattaaagtgtgcagagcggagtggcggtgtttaagccacctgtcgctgggactgtaatgaatgcgcatggccaccacccactgtcctctgtaatgttccgaacgtgagaccattggtcactactgagctgtggcgtgcgggatagtataaatcctgaggaccggcttgggctgccgacgattgctagtgaataatcatcttcgatataggtcacgcggtagtttgcttgattgtcttcactctgaaatggaatacctgggaagctaacctttaatgaagcattggaaactactttagcgctgccttcaataactgaaggcccaaagaaagtgccacacttatttgttacagagattgtgtccgagtcgatcacgccgtaatcagcggtaacgtcatgtgagcactgtaaagagaatggttggggaattgctgcgacttgataccacttgcctttgtagcgttctaggtcaatgctattttcaatttcgggcagcgctaggttttcaggaaccgaacttaggttagatacctgcgaggagccacctgcaagtcgtccgccgtcaaaaatgtcttgggcttgtgccgtggatatcccgaaaagtgaaatggctgcgagtagtgctgtggtgacaagtttgcttgaaatgcgcataaagcaaatcctttcttcatgtttatattaactcaatagttattacttctaaaagtatagtagatagttgtggatgggtgaagaatttcatagaaatcgcactcgattcactaaagacccaagagtaaaatcccaggatttgcttatacttgcgctcatggataatcaacttcgtcccactttgcattatcaagctcaaaacccgcaccctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattctagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 26 - protein, 373 aa, Bacillus subtilis; identical to SEQ ID NO: 2

SEQ ID NO: 27 - protein, 374 aa, Bacillus licheniformis
MTEHVQTTLAQIGNRSDEITGTVNPPVYFSSAYRHKGIGESTGFDYIRTKNPTRQLVEDAIAKLEGGTRGFAFSSGMAAIQTIMALFQSGDELIVSSDLYGGTYRLFENEWKKYGLRFLYDDFSDEDCIKSKITDNTKALFVETPTNPLMQEADIQKIAQIAKENDLLLIVDNTFYTPVLQRPIELGADLVIHSATKYLGGHNDLLAGLVVAKGEELSEEMFQHQNAIGAVLSPFDSWLLMRGLKTLALRMRQHQENARELAAFLEEQEEIADVLYPGKGGMLSFRVQKEEWVNPFLKNLKTICFAESLGGVESFITYPATQTHMDIPEDIRIANGVCNRLLRFSVGIEHVSDLKQDLKAALEKVKGEAVPHES

SEQ ID NO: 28 - protein, 387 aa, Bacillus licheniformis
MKKGFLLFKGWCHMTEHVQTTLAQIGNRSDEITGTVNPPVYFSSAYRHKGIGESTGFDYIRTKNPTRQLVEDAIAKLEGGTRGFAFSSGMAAIQTIMALFQSGDELIVSSDLYGGTYRLFENEWKKYGLRFLYDDFSDEDCIKSKITDNTKALFVETPTNPLMQEADIQKIAQIAKENDLLLIVDNTFYTPVLQRPIELGADLVIHSATKYLGGHNDLLAGLVVAKGEELSEEMFQHQNAIGAVLSPFDSWLLMRGLKTLALRMRQHQENARELAAFLEEQEEIADVLYPGKGGMLSFRVQKEEWVNPFLKNLKTICFAESLGGVESFITYPATQTHMDIPEDIRIANGVCNRLLRFSVGIEHVSDLKQDLKAALEKVKGEAVPHES

SEQ ID NO: 29 - protein, 367 aa, Geobacillus kaustophilus
MEKLETLLAQIGNRSETVTGTVNPPVYFSTAYRHAGIGESTGFDYIRTGNPTRKIVEEAIARLEGGDQGYAFSSGMAAIQTVLALFESGDEFLVSADLYGGTYRLFERGWRKYGLGFHYVDFADLAAVEACITEKTKAIFLETPTNPLMHETDIRAVSEFAKRHGLLLIVDNTFYTPVLQRPIEQGADIVIHSATKYLGGHNDVLAGLVVAKGEELCQRLAEYQNAIGAVLSPFDSWLLIRGMKTLALRMRQHEENAKRISAFLREHEDVTDVLYPGRGGMLSFRIADEKWVNGFLKSLRLITFAESLGGVESFITYPATQTHADIPEEIRIQNGICNRLLRFSVGIEHADDLIADLAQALKNMKEV

SEQ ID NO: 30 - protein, 378 aa, Bacillus halodurans
MNRKELETALVQIGNRMDDRTGAINTPVYFSTAYRHSGIGESTGYDYARTGNPTREVLEKAIATLENGDQGFACSSGMAAIQTVFSLFQSGDEIIASQDLYGGTYRLFEGGWKKWGLSFSYADPRNLAALEQQITEKTRALFIETPTNPLMQEANIRELAALANKHGLLLIVDNTFYTPLLQQPLNEGTHIVIHSASKYLGGHNDVIAGLIVAKGQELCEQIAYYHNGIGGTLSAFDSWLLIRGMKTLALRMEQHQNNARAIASYLEKHEGVTDVLYPGRGGMLSFRIQSESWVNPFLQSLKLISFAESLGGVESLMTYPATQTHADIPEDVRIANGVCNRLLRFSVGIEHVGDLIADLDQAFNRVIEQSAVKGSEAQ

SEQ ID NO: 31 - protein, 370 aa, Bacillus cereus
MSTIETKLAQIGNRSETTTGTVNPPVYFSTAYRHEGIGKSTGFDYSRTGNPTRGLLEQAIADLEYGEQGYACSSGMAAVLLVLSLFRSGDELIVSEDLYGGTYRLFSEHEKKWNVRCRYVNTQSIKQIEQAITTETKAIFIETPTNPLMQVTDIAAVATVAKRHGLLLIVDNTFYTPYIQQPLTEGADIVLHSATKYLGGHNDVLSGLVVAKGKELCEEIAHYHNASGAVLSPFDSWLLIRGMKTLALRMRQHEENAKAVVAYLNDEDGVTDVFYPGRGGMISFRLKDEEWINPFLQSLSLITFAESLGGVESLMTYPATQTHADIPEEIRTANGVCNRLLRFSVGIENSNDLIQDLQQAIKLVKEGVRI

SEQ ID NO: 32 - protein, 370 aa, Bacillus cereus
MSTIETKLAQIGNRSETTTGTVNPPVYFSTAYRHEGIGQSTGFDYSRTGNPTRGLLEQAIADLEYGEQGYACSSGMAAVLLVLSLFRSGDELIVSEDLYGGTYRLFSEHEKKWNVRCRYVNTQSIKQIEQAITTETKAIFIETPTNPLMQVTDIAAVATVAKRHGLLLIVDNTFYTPYIQQPLTEGADIVLHSATKYLGGHNDVLSGLVVAKGKELCEEIAHYHNASGAVLSPFDSWLLIRGMKTLALRMRQHEENAKAVVAYLNDEDGVTDVFYPGRGGMISFRLQDETWINPFLQSLSLITFAESLGGVESLMTYPATQTHADIPEDIRTANGVCNRLLRFSVGIENSNDLIQDLQQAIKLVKEGVRI

SEQ ID NO: 33 - protein, 370 aa, Bacillus thuringiensis
MSTIETKLAQIGNRSETTTGTVNTPVYFSTAYRHEGIGKSTGFDYSRTGNPTRGLLEQAIADLEYGEQGYACSSGMAAVLLVLSLFRSGDELIVSEDLYGGTYRLFSEHEKKWNVRCRYVNTQSIKQIEQAITTETKAIFIETPTNPLMQVTDIAAVATVAKRHGLLLIVDNTFYTPYIQQPLTEGADIVLHSATKYLGGHNDVLSGLVVAKGKELCEEIAHYHNASGAVLSPFDSWLLIRGMKTLALRMRQHEENAKAVVAYLNDEDGVTDVFYPGRGGMISFRLKDETWINPFLQSLSLITFAESLGGVESLMTYPATQTHADIPEEIRTANGVCNRLLRFSVGIENSNDLIQDLQQAIKLVKEGVRI

SEQ ID NO: 34 - protein, 370 aa, Bacillus anthracis
MSTIETKLAQIGNRSETTTGTVNPPVYFSTAYRHEGIGKSTGFDYSRTGNPTRGLLEQAIADLEYGEQGYACSSGMAAVLLVLSLFRSGDELIVSEDLYGGTYRLFSEHEKKWNVRCRYVNTQSIKQIEQAITTETKAIFIETPTNPLMQVTDIAAVATVAKRHGLLLIVDNTFYTPYIQQPLTEGADIVLHSATKYLGGHNDVLSGLVVAKGKELCEEIAHYHNASGAVLSPFDSWLLIRGMKTLALRMRQHEKNAKAVVAYLNDEDGVTDVFYPGRGGMISFRLKDETWINPFLQSLSLITFAESLGGVESLMTYPATQTHADIPEEIRTANGVCNRLLRFSVGIENSNDLIQDLQQAIKLVKEGVRI

SEQ ID NO: 35 - protein, 370 aa, Bacillus cereus
MSTIETKLAQIGNRSETTTGTVNPPVYFSTAYRHEGIGKSTGFDYSRTGNPTRGLLEQAIADLECGEQGYACSSGMAAVLLVLSLFRSGDELIVSEDLYGGTYRLFSEHEKKWNVRCRYVNTQSIKQIEQAITTKTKAIFIETPTNPLMQVTDIAAVATVAKRHGLLLIVDNTFYTPYIQQPLTEGADIVLHSATKYLGGHNDVLSGLVVAKGKELCEEIAHYHNASGAVLSPFDSWLLIRGMKTLALRMRQHEENAKAVVAYLNDEDGVTDVFYPGRGGMISFRLKDETWINPFLQSLSLITFAESLGGVESLMTYPATQTHADIPEEIRTANGVCNRLLRFSVGIENSNDLIQDLQQAVKLVKEGVRI

SEQ ID NO: 36 - protein, 370 aa, Bacillus cereus
MSTIETKLAQIGNRSETTTGTVNPPVYFSTAYRHEGIGKSTGFDYSRTGNPTRGLLEQAIADLEYGEQGYACSSGMAAVLLVLSLFRSGDELIVSEDLYGGTYRLFSEHEKKWNVRCRYVNTQSIKQIEQAITTETKAIFIETPTNPLMQVTDIAAVATVAKRNGLLLIVDNTFYTPYIQQPLTEGADIVLHSATKYLGGHNDVLSGLVVAKGKELCEEIAHYHNASGAVLSPFDSWLLIRGMKTLALRMRQHEENAKAVVAYLNDEDGVTDVFYPGRGGMISFRLKDEAWINPFLQSLSLITFAESLGGVESLMTYPATQTHADIPEEIRTANGVCNRLLRFSVGIENSNDLIQDLKQAIKLVKEGVRI

SEQ ID NO: 37 - protein, 369 aa, Pasteurella multocida
MTQHYSIETLLAQAGNRTDERTGAVSTPIFLSTAYAHHGIGESTGYDYTRTKNPTRSVLEETIAKLEGGERGFACASGMAAIQLIMSLFTSPDEWIVSRDVYGGTYRLLDFAYKNTQGVKPVYVNTSEVSCIEAAITSNTKAIFVETPSNPLMEECDVAAIAKIAKKHNLLLIVDNTFLTPVLFRPIEAGADIVIHSGTKYLAGHNDALVGLVVAKGEELCQRLFYIQNGAGAVLSPFDSWLTIRGMKTLALRMERHEQNAKQLAAFLASQPQVKNVLYPNKGGMLSFRLHEAHWVNPFLKALKLITFAESLGGTESFITYPATQTHMDIPEEERIARGVCNCLLRFSVGLENVEDIKADLLQAFAQLN

SEQ ID NO: 38 - protein, 369 aa, Haemophilus somnus
MTQQYALDTLLAQAGNRTDERTGAVSTPIFLSTAYAHHGIGESTGFDYTRTKNPTRTVLEETIAKLEGGDRGFACSSGMAAIQLLMNLFASPDEWIVSSDVYGGTYRLLDFAHKNIHGVKPVYVNTASSEEIEKAITENTKAIFVETPSNPLMEECDVAEIAKIAKKYNLLLIVDNTFLTPVLFRPMEHGADIVIHSGTKYIAGHNDTLVGLIVAKGQEICDRLYYIQNGAGPVLSPFDSWLTIRGMKTLALRMERHQKNAQELANFLREQPQVKDVLYPNKGGMLSFRLQNENWVNPFLKAMKLITFAESLGGTESFITYPATQTHMDIPEVERVARGVCNRLLRFSVGLENVEDIKADLLQAFSQLK

SEQ ID NO: 39 - protein, 369 aa, Mannheimia succiniciproducens
MTQNYSIETILAQAGNKSDARTGAVSTPIFLSTAYGHRGIGESTGFDYTRTKNPTRLVLEETIAKLENGDQGFAFSSGMAAIQVLMTLFTAPDEWIVSSDVYGGTYRLLDFAYKNNNSVKPVYVNTASVEAIETAITPNTKAIFVETPSNPLMEECNVTEIAKIAKKYNLLLIVDNTFLTPVFSRPLDLGADIVIHSATKYLAGHNDTLAGLVVAKGQALCERIFYIQNGAGAVLSPFDSWLTIRGLKTLALRMERHQANAAAIAEFLKAQPQVKDVLYPNKGGMLSFRLQDENWVNPFLKAINLITFAESLGGTESFITYPTTQTHMDIPAEERIARGVTNDLLRFSVGLENVEDIKADLAQAFAQFK

SEQ ID NO: 40 - protein, 369 aa, Haemophilus somnus
MTQQYALDTLLAQTGNRTDERTGAVSTPIFLSTAYGHHGIGESTGFDYTRTKNPTRTVLEETIAKLEGGDRGFACSSGMAAIQLLMNLFASPDEWIVSSDVYGGTYRLLDFAHKNIHGVKPVYVNTASSEEIEKAITENTKAIFVETPSNPLMEECDVAEIAKIAKKYKLLLIVDNTFLTPVLFRPMEHGADIVIHSGTKYIAGHNDTLVGLIVAKGQEICNRLYYIQNGAGPVLSPFDSWLTIRGMKTLALRMERHQKNAQELANFLREQPQVKDVLYPNKGGMLSFRLQNENWVNPFLKAMKLITFAESLGGTESFITYPATQTHMDIPEVERVARGVCNRLLRFSVGLENVEDIKADLLQAFSQLK

SEQ ID NO: 41 - protein, 381 aa, Haemophilus influenzae
MRSIFSLFLEDVMTQQYAIDTLLAQAGNRSDERTGAVSAPIFLSTAYGHCGIGESTGFDYTRTKNPTRTVLEETIAKLENGDRGFAFSSGMAAIQVLMTLFTAPDEWIVSSDVYGGTYRLLDFSYKNNNSVKPVYVNTASASAIEAAINPNTKAIFIETPSNPLMEECDVVEIAKLAKKHNLMLIVDNTFLTPVLSRPLDLGADVVIHSGTKYIAGHNDALVGLIVAKGQELCDRIAYIQNGAGAVLSPFDSWLTIRGMKTLSLRMKRHQENAQAIAEFLKAQPQVESVLYPNKGGMLSFRLQDEAWVNTFLKSIKLITFAESLGGTESFITYPATQTHMDIPESERVARGITNTLLRFSVGIEDVEDIKADLLQAFANLK

SEQ ID NO: 42 - protein, 369 aa, Haemophilus influenzae
MTQQYAIDTLLAQAGNRSDERTGAVSAPIFLSTAYGHCGIGESTGFDYTRTKNPTRTVLEETIAKLENGDRGFAFSSGMAAIQVLMTLFTAPDEWIVSSDVYGGTYRLLDFSYKNNNSVKPVYVNTASASAIEAAINPNTKAIFIETPSNPLMEECDVVEIAKLAKKHNLMLIVDNTFLTPVLSRPLDLGADVVIHSGTKYIAGHNDALVGLIVAKGQELCDRIAYIQNGAGAVLSPFDSWLTIRGMKTLSLRMKRHQENAQAIAEFLKAQPQVESVLYPNKGGMLSFRLQDEAWVNTFLKSIKLITFAESLGGTESFITYPATQTHMDIPESERVARGITNTLLRFSVGIEDVEDIKADLLQAFANLK

SEQ ID NO: 43 - protein, 369 aa, Haemophilus influenzae
MTQQYAIDTLLAQAGNRSDERTGAVSAPIFLSTAYGHCGIGESTGFDYTRTKNPTRTVLEETIAKLENGDRGFAFSSGMAAIQVLMTLFTAPDEWIVSSDVYGGTYRLLDFSYKNNNSVKPVYVNTASASAIEAAINPNTKAIFIETPSNPLMEECDVVEIAKLAKKHNLMLIVDNTFLTPVLSRPLDLGADVVIHSGTKYIAGHNDALVGLIVAKGQELCDRIAYIQNGAGAVLSPFDSWLTIRGMKTLSLRMKRHQENAQAIAEFLKAQPQVESVLYPNKGGMLSFRLQDEAWVNTFLKSIKLITFAESLGGTESFITYPATQTHMDIPEPERVARGITNTLLRFSVGIEDVEDIKADLLQAFANLK

SEQ ID NO: 44 - protein, 369 aa, Haemophilus influenzae
MTQQYAIDTLLAQAGNRSDERTGAVSAPIFLSTAYGHCGIGESTGFDYTRTKNPTRTVLEETIAKLENGDRGFAFSSGMAAIQVLMTLFTAPDEWIVSSDVYGGTYRLLDFSYKNNNSVKPVYVNTAFASEIEAAINPNTKAIFIETPSNPLMEECDVVEIAKLAKKHNLMLIVDNTFLTPVLSRPLDLGADVVIHSGTKYIAGHNDALVGLIVAKGQELCDRIAYIQNGAGAVLSPFDSWLTIRGMKTLSLRMKRHQENAQAIAEFLKDQPQVESVLYPNKGGMLSFRLQDEAWVNTFLKSIKLITFAESLGGTESFITYPATQTHMDIPESERVARGITNTLLRFSVGIEDVEDIKADLLQAFANLK

SEQ ID NO: 45 - protein, 369 aa, Haemophilus influenzae
MTQQYAIDTLLAQAGNRSDERTGAVSAPIFLSTAYGHCGIGESTGFDYTRTKNPTRTVLEETIAKLENGDRGFAFSSGMAAIQVLMTLFTALDEWIVSSDVYGGTYRLLDFSYKNNNSVKPVYVNTASASEIEAAINPNTKAIFIETPSNPLMEECDVVEIAKLAKKHNLMLIVDNTFLTPVLSRPLDLGADVVIHSGTKYIAGHNDALVGLIVAKGQELCDRIAYIQNGAGAVLSPFDSWLTIRGMKTLSLRMKRHQENAQAIAEFLKDQPQVESVLYPNKGGMLSFRLQDEAWVNTFLKSIKLITFAESLGGTESFITYPATQTHMDIPETERVARGITNTLLRFSVGIEDVEDIKADLLQAFANLK

SEQ ID NO: 46 - protein, 368 aa, Bacillus clausii
MEKRAETILAQIGNRRDEHTGAVNTPVYFSTAYRHPGIGESTGYDYARTGNPTRDVLEKAIAELEEGERGFATSSGMAAVQIVLSLFEQGDGIICSKDLYGGTYRLFEGGWTRWGVSFTYVDPRNLQEVEQAIHSNVKAIFIETPTNPLMQEASIPALAALAKKHDLLLIVDNTFYTPLLQKPLTEGADIVIHSATKYLGGHNDVVAGLIVAKGADLCERLAYYHNGAGGILSAFDSWLLIRGMKTLALRMAKHEENAKKVVHALEQTDGIVDVLYPGRGGMLSFRVQNEAWVNPLLKHLQLISFAESLGGVESLMTYPATQTHADIPEEIRLANGVDNGLLRFSVGIENGEDIVADLLQAIAAAKKS

SEQ ID NO: 47 - protein, 380 aa, Actinobacillus pleuropneumoniae
MKMTKYSNIETTLVQLGNRTDPRTGAVATPIVLSTAYGRGGLGESTGWDYIRTKNPTRAVLEQGIADLEGGDAGFAMASGMAAIQLVMSLFKAPDEWIISSDVYGGSYRLFDFSHKHHNTVKPVYVNTADLAAIEAAITPNTKAIFVETPSNPLMEECDVDAISKIAKKHNLMLIVDNTFLTPILFRPIEHGADIVIHSATKYLSGHNDVLAGLIVAKDSEATKNEAGQKLSERLFYFQNCAGAVLSPFDSYLAVRGLKTLALRMERHQSNATELARFLSEQPEIECVLYSGKSGMLSFRLQKEEWVPKFLKAIKLITFAESLGGTESFITYPSTQTHMDIPEAERIARGITNNLLRFSVGLEHVEDLKVDLRQAFGQLK

SEQ ID NO: 48 - protein, 374 aa, Listeria monocytogenes
MAKLKQETIAAQIGNRKCERTGAVNMPVYFSTAYQHADLGVSTGYDYTRTGNPTRDALQEALAELENGTHAFATSSGMSAIQLVFQLFKTGEHIISSQDLYGGTFRYFEQFGAQYNIGFSYWDGAEIADLEKLVRPETKAIFIETPTNPLMQETDIAAVSEWAGAHDLLVIVDNTFYTPILQQPLTLGADIVIHSATKYLGGHNDVLAGAVIVKEERLGNFFFNQLNATGAVLSPFDSWLLIRGLKTLVLRVRQHQANAEKIAAFLESHELVEEVRYPGRGGMISFFIQDAALVSPLLKELELFTFAESLGGVESLITYPTTQTHADIPEELRNSYGLTDKLLRISVGIEASEDLIADLSKALDAVLEGVSARG

SEQ ID NO: 49 - protein, 374 aa, Listeria monocytogenes
MAKLKQETIAAQIGNRKCERTGAVNMPVYFSTAYQHADLGVSTGYDYTRTGNPTRDALQEALAELENGTHAFATSSGMSAIQLVFQLFKTGEHIISSQDLYGGTFRYFEQFGAQYQIGFSYWDGAEITDLEKLIRPETKAIFIETPTNPLMQETDIATVAKWAHAHDLLVIVDNTFYTPVLQQPLSLGADIVIHSATKYLGGHNDVLAGAVIVKEEKLGKFFFDQLNATGTVLSPFDSWLLIRGLKTLVLRVRQHQANAQKIAAFLEEHKLVEEVRYPGRGGMISFFIRDAALVSPLLKELELFTFAESLGGVESLITYPTTQTHADIPVELRNSYGLTDKLLRISVGIEASEDLIADLSKALDAVLEEVSARG

SEQ ID NO: 50 - protein, 374 aa, Listeria monocytogenes
MAKLKQETIAAQIGNRKCERTGAVNMPVYFSTAYQHADLGVSTGYDYTRTGNPTRDALQEALAELENGTHAFATSSGMSAIQLVFQLFKTGEHIISSQDLYGGTFRYFEQFGAQYQIGFSYWDGAEIADLEKLIRPETKAIFIETPTNPLMQETDIATVAKWAHAHDLLVIVDNTFYTPVLQQPLSLGADIVIHSATKYLGGHNDVLAGAVIVKEEKLGKFFFDQLNATGTVLSPFDSWLLIRGLKTLVLRVRQHQANAQKIAAFLEEHKLVEEVRYPGRGGMISFFIRDAALVSPLLKELELFTFAESLGGVESLITYPTTQTHADIPVELRNSYGLTDKLLRISVGIEASEDLIADLSKALDAVLEEVSARG

SEQ ID NO: 51 - protein, 374 aa, Listeria innocua
MAKLKQETIAAQIGNRKCERTGAVNMPVYFSTAYQHADLGVSTGYDYTRTGNPTRDALQEALAELENGTHAFATSSGMSAIQLVFQLFKTGEHIISSQDLYGGTFRYFEQFGAQYQIGFSYWDGANVNDLEKLVRPETKAIFIETPTNPLMQETDIASVSRWAHANDLLVIVDNTFYTPVLQQPLTLGADIVVHSATKYLGGHNDVLAGAVIVKEEWLGKFFFDQLNATGTVLSPFDSWLLIRGLKTLVLRVRQHQANAEKIAAFLEEHELVEEVRYPGRGGMISFFIKDAALVSPLLKELELFTFAESLGGVESLITYPTTQTHADIPLELRNSYGLTDKLLRISVGIEASEDLIADLSKALDAVLKGVTARG

SEQ ID NO: 52 - protein, 384 aa, Clostridium acetobutylicum
MGEIDCRNFETKAVHGESGFESRTGAISYPIYQSSTFRHEGLNKGTGYDYSRTGNPTRDEVEKTVAALENGRACLAYSSGMAAISSVLTIFKGGDHIIVSDDLYGGTYRIFEEIYEHYGIEVTYTDTTSTENIEKELRENTKAIYLETPTNPLMKITDIREVSKLAKEHNTLLIVDNTFMTPYYQKPLELGADIVLHSGTKYLCGHNDALAGFVILNDERLIEKLRFIQNSVGAVLAPFDSWLILRGIKTLHIRLDRQQENAIKIANFLKKHKKITKVLYPGLEEHVGHDILKSEASGFGAMISFYVDSKETVEKVLESVKVIIFAESLGGVESLITYPYTQTHADIPDDIRKRLGVTDKLLRFSVGIENVDDLIKDLDKALQA

SEQ ID NO: 53 - protein, 359 aa, Symbiobacterium thermophilum
MKLDTVLVHAGVRRDPAYGAVSVPVYQSATFQHPALGESTGYDYSRSGNPTRAALEEALARAEGGARALAFASGMAALTCALGLFGPGDHLVVTEDLYGGTYRLLEQVLALPHTYADTADLDAVRAAIRPDTRAVLVESLTNPRMKRADVAALAGLCRAHGLLLIVDNTFLTPWLCRPLELGADIVVHSATKYLAGHNDVVAGALITRDTALGDRLSFLQNAVGSVLGPQDSWLVLRGLKTLALRMERHQQNAARIAAWLREHPRVEEVLYPGVGGMLSFTVTEPSLVPQVLRRVKLILFAESLGGVESLITFPWTQTHADMPEEVRRRLGITDRLLRLSVGIEDADDLIADLAQALEG

SEQ ID NO: 54 - protein, 373 aa, Lactobacillus plantarum
MTKQAEKLHIETRLAQAGNRSDDEKTGAISAPIYLSTAYRHAGLGQSTGFDYPREAQPTRCILERVLAEMEHGIAAYALTSGMAAIQLVFTLFNSGDKIIVSDDLYGGSYRFFDLLHDHYHLDFAVWDGQDQATLAALADDQTVALWLETPSNPTMKVIDITATAATAHAHDLKLIVDNTFYTPLIQKPLDLGADIVVHSATKYLAGHNDVLAGAVVVKSQADADALEFNLVTTGAVLDPFDAWLLLRSLKTLPLRLHQQEANAQELVTVLEADEHVERVLYSGRGGMISFYLATGTDVDTFLRALNVISFAESLGGVESLLTVPAVQTHADLTEEQRQSKGITANLLRLSVGIENSADLAADLKQALIRATK

SEQ ID NO: 55 - protein, 367 aa, Staphylococcus epidermidis
MKDTDLAQIALTQDHTGAIANPIYLSTAYQHPHLGESTGYDYTRTKNPTRTAFEEAFAQLEKGIASFATSSGMAAIQLICNIFKPGDEILVAFDLYGGTFRLFDFYEKQYGLKFKYVDFLNYEEVEKNITPQTRALFIEPISNPQMIEIDVETYYILSKKHQLLTIIDNTFLTPYLSTPLEEGADIVLHSATKYIGGHNDVLAGVVTVKDAQLAEQLNQFHNMIGATLSPLDSYLLQRGLKTLHLRIERSQENAQKLAQRCRQSDSIDEVLYSGRTGMLSLRLNQAYSVAKFLENLEICIFAESLGGTETFITFPYTQTHVDMPDEEKDKRGIDEYLIRLSVGIEDYNDIEADIIQALENSKVGVIS

SEQ ID NO: 56 - protein, 367 aa, Staphylococcus epidermidis
MKDTDLAQIALTQDHTGAIVNPIYLSTAYQHPHLGESTGYDYTRTKNPTRTAFEEAFAQLEKGIASFATSSGMAAIQLICNIFKPGDEILVAFDLYGGTFRLFDFYEKQYGLKFKYVDFLNYEEVEKNITPQTRALFIEPISNPQMIEIDVEPYYILSKKHQLLTIIDNTFLTPYLSTPLEEGADIVLHSATKYIGGHNDVLAGVVTVKDAQLAEQLNQFHNMIGATLSPLDSYLLQRGLKTLHLRIERSQENAQKLAQRCRQSDSIDEVLYSGRTGMLSLRLNQAYSVAKFLENLEICIFAESLGGTETFITFPYTQTHVDMPDEEKDKRGIDEYLIRLSVGIEDYNDIEADIIQALENSKVGVIS

SEQ ID NO: 57 - protein, 386 aa, Clostridium thermocellum
MMKVGNVSNYSISTKVVHGSKCYDPHTGAVSFPIYQSATFRHPALYQTTGYDYSRLQNPTREELENTIANIENGKFGFAFSSGMAAVSTILSLFSPKDHIIVSDDLYGGTYRLFEEIYKKYGLEFSYVNTSRIQDIEEAVKENTKAFFIETPTNPMMKVADLKTISRFAKDRKILLIVDNTFLTPYFQRPLELGADIVVHSGTKYLGGHNDTLAGLVVVNDEELAERIKLIQKSEGAVLSPFDSWLILRGIKTLGVRLEKQQENAMKIAKWLCTHKNVTKVNYVGLPDHEGYEISKSQASGFGAMISFNVKDVQTVEKVLSKVQLVMFAESLGGVESLITYPAVQTHAAIPEEMRNRIGVTDTLLRLSVGIEDADDIIADLEQALE

SEQ ID NO: 58 - protein, 378 aa, Moorella thermoacetica
MRLATELVQLGVGYDSKTGAISTPIYQSATFRHPALGQSTGFDYSRTGNPTRQVLEEGLAGLEGGCRALAFASGMAAITAVLCLFRPGDHLVVSEDLYGGTYRLLNQVAVPLGLEFSLVDTTDLAALAASIRNNTKGIFLETPTNPLMKITDIAAVVALARQRGLLTIVDNTFMTPYLQRPLELGADLVVHSATKYLGGHNDVVMGAAIAAREDLSERLAFIQNTIGAIPGPQDCWLVIRGLKTLAVRLERAQANAFELARWLAEHPLVTRVYYPGLPHHPGHEICKKQSSGFGAMLSFEVKHAGLVEQILQRLKIISFAESLGGVESLITFPERQTHAEIPAEMRLKLGINDRLLRLSVGLEDLNDLKADLDQALAC

SEQ ID NO: 59 - protein, 364 aa, Streptococcus thermophilus
MTQDYQLETILAHAGINSDEATGALASPIHFSTTYQHPEFGQSTGFDYTRTKNPTRATVEKTLAAIEKADYAIATSSGMSAIVLAFEIFPVGSKVVAARDLYGGSFRWFNDKEKEGRFFFEYTNTEDEMIAAIAEDTDIVYIETPTNPLMIEFDIEKVAQTAHEKGAVVIVDNTFYSPIYQTPITQGADIVVHSATKYLSGHNDVLAGVVVTSNPEFYDKLYYNLNTTGPNLSPFDSYMLMRGLKTLKLRMEASTANAKEVVAFLEKSPAVKEVLYPGKGGMISFKVANQDKIPTIINTLKVFTFAESLGGVESLITYPATQTHADIPSEVRASYGLTDDLLRLSIGIEAAEDLIADLENALSL

SEQ ID NO: 60 - protein, 364 aa, Streptococcus pneumoniae
MSKELHINTILAQAGIKSDEATGALVTPLHFSTTYQHPEFGRSTGFDYTRTKNPTRSKAEEVLAAIESADYALATSSGMSAIVLAFSVFPVGSKVLAVRDLYGGSFRWFNQVEQEGRFHFTYANTEEELIAELEKDVDVLYIETPTNPLMLEFDIEKLAKLAHAKGAKVVVDNTFYSPIYQRPIEDGADIVLHSATKYLAGHNDVLAGVVVTNSLELYEKLFYNLNTTGAVLSPFDSYQLLRGLKTLSLRMERSTANAQEVVAFLKDSPAVKEVLYTGRGGMISFKVADETRIPHILNSLKVFSFAESLGGVESLITYPTTQTHADIPAEVRHSYGLTDDLLRLSIGIEDARDLIADLRQALEG

SEQ ID NO: 61 - protein, 377 aa, Geobacter sulfurreducens
MNIATQAAQIGLDWDTRTGAVTVPIYQTATFRHPGLGQSTGYDYSRSGNPTRQALEEGIARLDGGARGFAYASGMAAIANLLLLFKSGDHLVVTEDLYGGTCRLFDQILVQYGLSFTYVDTSDPEAVRDAIRPETRALFVESLTNPLLKVADIAALSTLCRERGLLCIVDNTFLTPYLLRPLDLGADITVYSGTKYLSGHNDTVSGLVVVKEPALAERVYFLQNSVGAVLGPQDAWLTIRGMKTLSVRLDRQQENAGRIAEWLARHPRVPRVFYPGLPGHPGHELLARQARGFGAMIAFEVDDKALVERLLLKTELISFAESLGGVETLITFPQVQTHADIPPEVRERLGINDVLLRLSVGIEDADDLIADLSRAFA

SEQ ID NO: 62 - protein, 391 aa, Geobacter metallireducens
MNIATQTAQIGLEWDTRTGAVTVPIYQTATFRHPGLGQSTGYDYTRSGNPTRQALEEGIARLEGGARGFAYASGMAAIANLLLLFKKGDHLVVTEDLYGGTCRLFDQIFTQYELSFTYVDTSDIKAVRAAIRPETKALFVESLTNPLLKVADIAALSALCRERGLLHIVDNTFLTPYLLRPFDHGADITVYSATKYLAGHNDTVSGLVAVKDPQLAERVYFLQNSVGAVLGPQDSWLTIRGMKTLSVRLDRQQENAGRVAQWLSNHPRVRKVYYPGLSGHPGHPGHELLARQARGFGAMIAFEVDEHALVERLLLKTEVISFAESLGGVETLITFPQVQTHADIPPELRQRLGINDVLLRLSVGIEDADDLIDDLAQAFEGGDQGSGTGDR

SEQ ID NO: 63 - protein, 364 aa, Streptococcus pneumoniae
MSKELHINTILAQAGIKSDEATGALVTPLHFSTTYQHPEFGRSTGFDYTRTKNPTRSKAEEVLAAIESADYALATSSGMSAIVLAFSVFPVGSKVLAVRDLYGGSFRWFNQVEQEGHFHFTYANTEEELIAELEKDVDVLYIETPTNPLMLEFDIEKLAKLAHAKGAKVVVDNTFYSPIYQRPIEDGADIVLHSATKYLAGHNDVLAGVVVTNSLELYEKLFYNLNTTGAVLSPFDSYQLLRGLKTLSLRMERSTANAQEVVAFLKDSPAVKEVLYTGRGGMISFKVADETRIPHILNSLKVFSFAESLGGVESLITYPTTQTHADIPAEVRHSYGLTDDLLRLSIGIEDARDLIADLRQAIEG

SEQ ID NO: 64 - protein, 364 aa, Streptococcus anginosus
MDKKLQLDTILAHAGIKTDEATGALTTPLHFSTTYQHPEFGKSTGYDYTRTKNPTRSSLEKTLAAIEHADYALATSSGMSAIVLAFSVFPIGSRVIAVRDLYGGSFRWFHQVEQEGRFHFTYANTEEELLAALTEDIDVVYLETPTNPLMLEFDVAFIAEKAHAKGAKVIVDNTFYTPIYQRPLENGADLVLHSATKYLGGHNDVLAGALMTSDKELYENLFYNLNTTGAVLSPFDSYLLLRGLKTLSLRMERSTKNAQAVAAFLKDSPAVKEVLYPGKGGMISFKVKDEAVIPHLLNTLKVFTFAESLGGVESLITYPATQTHADIPAEVRKSYGLTDDLLRLSIGIEDSQDLITDLKFALEV

SEQ ID NO: 65 - protein, 364 aa, Streptococcus mutans
MTEDYKLDTILAHAGINTDKTTGALTAPIHLSTTYQHPQFGQSTGFDYTRTKNPTRTVLEETLAKIEKAKYALVTSSGMAALVLLFTGFPIGSKVVAARDLYGGSFRWFNEQEKAGRFSFVYTNTETDMIAAISDETDYVFIETPTNPLMIEFDISKVAQAAHKHGAKVIVDNTFYSPIYQNPLVLGADVVLHSATKYLSGHNDVLAGVLMTSDQEIYDKLFYDQNTTGPTLSPLDTYLLMRGLKTLKLRMEKATQNAKTVVAYLEKSPAVKEVLYTGKGGMISFKVVDEKKIPQILNHLQLFTFAESLGGVESLITYPATQTHLDIPEEVRHSYGLTDDLLRLSIGIEDAEDLIDDLKAALEA

SEQ ID NO: 66 - protein, 380 aa, Bacillus licheniformis
MKPKTKMIHGGITGDEKTGAVSVPIYQVSTYKQPRAGQHTGYEYSRTGNPTRTALESLIADVEGGAAGYAFGSGMAAITAVMMLFKSGDHIVLTDDVYGGTYRVMTKVLNRIGIEATFSDTSSIEDIEKAIKPNTKAIYVETPTNPLLKITDIKKTAETAKKHDLLLIVDNTFYTPYFQNPISLGADIVLHSATKYLGGHSDVVGGLVVAASKELAEEIHFIQNSTGGILGPQDSWLLMRGMKTLGLRMEAHEQNARKIAAFLDDHPAVKKVYYPGLPSHPGHELAKRQSTGFGGMISFDIGKEENVDLVLGRLKWFTIAESLGAVESLISVPARMTHASIPPERRLELGITDGLIRISAGVEDIDDLLEDLQQALAPLS

SEQ ID NO: 67 - protein, 362 aa, Lactococcus lactis
MDKRLDTLLAQVGIHQDEATGALVSPLHFSTTYQHPEFGQSTGFDYTRTKNPTRATLEEALASIESGQFALATSSGMAAIVLAFSVFPIGSKIVASRDLYGGSFRWFDEQEKEGRFYFSYAKTEKEMLELIDENTDIVYIETPTNPMMVKYNIEKIANKAQAYHAKVIVVNTFYTPIYQKPLELGADLVIHSATKYLSGHNDVLAGAVIVYDEELYERLLYQLNTTGAVLSPFDSYLVMRGLKTLSLRMERATKNAQKIVTFLKKLPSVKEVLYSGLGGMISLKVTDKTKIPAILNHLGVFTFAESLGGVESLITYPTAQTHHDIPLEIRESYGLTDDLLRLSIGIEDVRDLIEDLKEALEN

SEQ ID NO: 68 - protein, 367 aa, Staphylococcus aureus
MKDTQLAQITLTDDSTGAIANPIHLSTAYKHPKLGQSTGFDYTRTKNPTRSTFETCFAKLEHGIASFATSSGMSAIQLICNLFKPHDEILVSFDLYGGTFRLFEFYEQQYNIKFKYVDFTDYEQVEKEITDKTVALFIEPISNPQMIAIDVKPYYQLCKAKGLLSIIDNTFLTPYLSTPLAEGADIVLHSATKYIGGHNDVLAGVVTVKDESLAQQLFDFHNMTGATLSPIDSYLLLRGLKTLHLRIERAQSNARKLAKKCQSLQAIDEVLYSGQTGMLSLRLNKAYSVAKLLENLDICIFAESLGGTETLVTFPYTQTHVDMPDAEKDKRGIDEYLIRLSLGVENYEDIERDIIQALDKAQIGEIV

SEQ ID NO: 69 - protein, 367 aa, Staphylococcus aureus
MKDTQLAQITLTDDSTGAIANPIHLSTAYKHPKLGQSTGFDYTRTKNPTRSTFETCFAKLEHGIASFATSSGMSAIQLICNLFKPHDEILVSFDLYGGTFRLFEFYEQQYDIKFKYVDFTDYEQVEKEITDKTVALFIEPISNPQMIAIDVKPYYQLCKAKGLLSIIDNTFLTPYLSTPLAEGADIVLHSATKYIGGHNDVLAGVVTVKDESLAQQLFDFHNMTGATLSPIDSYLLLRGLKTLHLRIERAQSNARKLAKKCQSLQAIDEVLYSGQTGMLSLRLNKAYSVAKLLENLDICIFAESLGGTETLVTFPYTQTHVDMPDAEKDKRGIDEYLIRLSLGVENYEDIERDIIQALDKAQIGEIV

SEQ ID NO: 70 - protein, 367 aa, Staphylococcus aureus
MKDTQLAQITLTDDSTGAIANPIHLSTAYKHPKLGQSTGFDYTRTKNPTRSTFETCFAKLEHGIASFATSSGMSAIQLICNLFKPHDEILVSFDLYGGTFRLFEFYEQQYDIKFKYVDFTDYEQVEKEITDKTVALFIEPISNPQMIAIDVKPYYQLCKAKGLLSIIDNTFLTPYLSTPLAEGADIVLHSATKYIGGHNDVLAGVVTVKDESLAQKLFDFHNMTGATLSPIDSYLLLRGLKTLHLRIERAQSNARKLAEKCQSLQAIDEVLYSGQTGMLSLRLNKAYSVAKLLENLDICIFAESLGGTETLVTFPYTQTHVDMPDAEKDKRGIDEYLIRLSLGVENYEDIERDIIQALDKAQIGEIV

SEQ ID NO: 71 - protein, 383 aa, Helicobacter hepaticus
MKSTLDTLLIHGGATTDPRTGAVNIPIYQTSTYAQSALGEHLGYEYSRTKNPTRDGIESLIAQCEGGKFGFAFASGMAAIGTILSLFQSGDCIIISNNVYGGTFRILDKVFSHFNISYKIVDTRDLKALESAITPEVKAVLIETPANPLLSVTPLEQVAILAKKKGILSIVDNTFMTPYLQKPLELGIDIVMHSATKYLGGHSDLIAGLVVVNDSALAERIGFLQNSIGGVLAPFDSFLLIRGMKTLGVRLQRHCENALFLAQALSEHSGVEKVYYPGLKSDEGYQIQNSQARSGGGMLSFELKKNYDYRIFFKSTQTIVLAESLGGVESLLCHPASMTHASIPKDVRERMGISEHLIRLSVGIEYAQDLLDDLNQAIKKAKV

SEQ ID NO: 72 - protein, 381 aa, Enterococcus faecium
MHIQTKLIHGGISEDPTTGAVSVPIYQTSTYRQDGVGQPKQYEYSRSGNPTRFALEELIADLEGGVRGFAFSSGLSGIHAVFSLFQAGDHILLGDDVYGGTFRLFDKVLTKNGLEYTIIDTSNLDKIEQSIKPNTKALYLETPSNPLLKITDLEKSATLAHQHGLIVIADNTFATPYFQRPLDLGSDIVVHSGTKYLGGHSDVVAGLVTSNHKDLADQIGFYQNAIGAVLGPQDSWLLQRGIKTLSVRMEEHQKNAFVVADFLFSHPAVEKVYYPGLPDHELHGVAKQQMSGFSGMISFTLKNEESAIPFVESLQLFTLAESLGGVESLVEIPSVMTHASIPKEKREEAGIKDGLIRLSVGIEYGQDLINDLAQAFDRIKN

SEQ ID NO: 73 - protein, 378 aa, Anabaena variabilis
MEFETKAIHEGQQSDPQTGAVIVPIYLTSTYQQEAIGQHKGYEYSRTGNPTRNALEEALAAIENGEYGLAFASGLAATTTVLSLLKSCDHIVAGDDLYGGTYRLLERVVKNWGVTTTYVDIDDISNFAKAIQPNTKLIWVETPTNPLLKIIDIAALANIAEQNNLILVVDNTFASPYFQRPLDNGADIVVHSTTKYLGGHSDIIGGAVVTSNEQLYTELKFYQNAIGAVPSPFDSWLVLRGIKTLAVRMREHEKNALLLAQFLEQHPKVERVYYPGLPSHEQHQLAKSQMSGFGGMISLELKGDFADVEKFASRLQLFLLAESLGGVESLLCYPAKMTHGSLPQEERYKRGINDNLVRLSVGIENVLDLQADLENALS

SEQ ID NO: 74 - protein, 363 aa, Streptococcus suis
MTDYKIDTILAHTGINSDERTGALISPIHLSTTYQHPEFGQSTGYDYTRTKNPTRASLETTLAAIEKADYALATSSGMAALVLLFNGFPVGSQVVAARDLYGGSFRWFNEQESIGRFQFTYANTEEELIAAITEETDYVYLETPTNPLMVEFDIAKVSAIAHAKGAKVIVDNTFYSPIYQNPLVLGADVVLHSATKYLSGHNDVLAGALMTNDQDLYDKLFYDQNTSGPTLSPLDSYLLMRGLKTLSLRMERTTQNAQKIVAYLEKSPAVKQVYYTGKGGMISVKVVDESRIPHILNTLKVFTFAESLGGVESLITYPATQTHADIPAEIRHSYGLTDDLLRLSIGIEDAEDLIADLKTALEG

SEQ ID NO: 75 - protein, 380 aa, Lactococcus lactis
MTSIKTKVIHGGISTDKTTGAVSVPIYQTSTYKQNGLGQPKEYEYSRSGNPTRHALEELIADLEGGVQGFAFSSGLAGIHAVLSLFSAGDHIILADDVYGGTFRLMDKVLTKTGIIYDLVDLSNLDDLKAAFKEETKAIYFETPSNPLLKVLDIKEISAIAKAHDALTLVDNTFATPYLQQPIALGADIVLHSATKYLGGHSDVVAGLVTTNSKELASEIGFLQNSIGAVLGPQDSWLVQRGIKTLALRMEAHSANAQKIAEFLETSKAVSKVYYPGLNSHPGHEIAKKQMSAFGGMISFELTDENAVKDFVENLSYFTLAESLGGVESLIEVPAVMTHASIPKELREEIGIKDGLIRLSVGVEAIEDLLTDIKEALEKK

SEQ ID NO: 76 - protein, 15 aa, Artificial Sequence
  Description of Artificial Sequence: Synthetic amino acid motif
  MOD_RES (1): Asp or Gln
  MOD_RES (2): Leu, Ile, Val, Met or Phe
  MOD_RES (3)..(5): Any amino acid
  MOD_RES (6): Ser, Thr, Ala, Gly or Cys
  MOD_RES (7): Ser, Thr, Ala, Gly, Cys or Ile
  MOD_RES (10): Phe, Tyr, Trp or Gln
  MOD_RES (11): Leu, Ile, Val, Met or Phe
  MOD_RES (12): Any amino acid
  MOD_RES (14): His or Gln
  MOD_RES (15): Ser, Gly, Asn or His
XXXXXXXTKXXXGXX

(19) United States
(12) Patent Application Publication
(10) Pub. No.: US 2009/0221027 A1
(43) Pub. Date: Sep. 3, 2009

(54) Use of a bacillus meti gene to improve methionine production in microorganisms

(75) Inventors: Oskar Zelder, Speyer (DE); Andrea Herold, Ludwigshafen (DE); Corinna Klopprogge, Mannheim (DE); Hartwig Schröder, Nussloch (DE); R. Rogers Yocum, Lexington, MA (US); Mark Williams, Revere, MA (US)

Correspondence Address: LAHIVE & COCKFIELD, LLP;FLOOR 30, SUITE 3000, ONE POST OFFICE SQUARE, BOSTON, MA, 02109, US

(73) Assignee: BASF AG, Ludwigshafen (DE)

(21) Appl. No.: 11/988,977

(22) PCT Filed: Jul. 18, 2006
(86) PCT No.: PCT/US2006/027617

§ 371 Date: Apr. 10, 2009

Related U.S. Application Data
(60) Provisional application No. 60/700,557, filed on Jul. 18, 2005.
(60) Provisional application No. 60/713,905, filed on Sep. 1, 2005.

(51) Int. Cl. C12P 21/06; C12P 13/22; C12P 13/14; C12P 13/12; C12P 13/08; C12P 13/06; C12P 5/02; C12N 1/21; C12N 15/63; C07H 21/04

(52) U.S. Cl. 435/69.1; 435/108; 435/110; 435/113; 435/115; 435/116; 435/167; 435/252.32; 435/320.1; 536/23.7

(57) ABSTRACT

The present invention pertains to improved microorganisms and methods for the production of methionine and other sulfur containing fine chemicals using the metI gene from Bacillus subtilis or a gene related to metI. In some embodiments of the present invention, the metI gene or another gene is integrated in a fashion that allows for co-production of a water soluble compound such as methionine or other amino acid and a caortenoid compound.

### RELATED APPLICATIONS

This application claims the benefit of priority to U.S. Provisional Patent Application No. 60/700,557, filed on Jul. 18, 2005, and U.S. Provisional Patent Application No. 60/713,905, filed on Sep. 1, 2005, both entitled “Use of a Bacillus metI Gene to Improve Methionine Production in Microorganisms,” the entire disclosure of each of which is incorporated by reference herein.

Additionally, this application is related to U.S. Provisional Patent Application No. 60/700,698, filed on Jul. 18, 2005, and U.S. Provisional Patent Application No. 60/713,907, filed Sep. 1, 2005, both entitled “Use of Dimethyl Disulfide for Methionine Production in Microrganisms,” the entire disclosure of each of which is incorporated by reference herein.

This application is also related to U.S. Provisional Patent Application No. 60/700,699, filed Jul. 18, 2005, and U.S. Provisional Patent Application No. 60/714,042, filed Sep. 1, 2005, both entitled “Methionine Producing Recombinant Microorganism,” the entire disclosure of each of which is incorporated by reference herein.

### BACKGROUND OF THE INVENTION

The biosynthesis of sulfur-containing fine chemicals, for example, methionine, homocysteine, S-adenosylmethionine, glutathione, coenzyme A, coenzyme M, mycothiol, cysteine, biotin, thiamine, and lipoic acid, occurs in cells via natural metabolic processes. These compounds, collectively referred to as “sulfur-containing fine chemicals”, include organic acids, both proteinogenic and nonproteinogenic amino acids, vitamins, and cofactors, and are used in many branches of industry, including food, animal feed, cosmetics, and pharmaceutical industries. These compounds can potentially be produced on a large scale by means of cultivating microorganisms, such as bacteria, and in particular coryneform bacteria, that have been developed in order to produce and secrete large amounts of the substance desired.

There exists a need for improved production processes for sulfur-containing fine chemicals, such as methionine, due to the great importance of these chemicals across a wide range of industries.

### SUMMARY OF THE INVENTION

The present invention relates to improved microorganisms and methods (e.g., microbial biosyntheses, microbial fermentation) for the production of methionine and other fine sulfur containing chemicals. In particular, the present inventors have discovered that certain useful enzymes involved in methionine biosynthetic pathways in e.g. Bacillus are not subject to methionine feedback inhibition. More specifically, it is demonstrated herein that Bacillus metI gene, when expressed at higher than normal levels or expressed constitutively or introduced (via ,e.g., transformation) into a heterologous microorganism, allows for the increased production of methionine.

The present invention, therefore, further relates to recombinant microorganisms having the ability to more effectively produce methionine. These microorganisms may employ the transsulfuration pathway or the direct sulfhydrylation pathway, wherein, introducing a gene, such as Bacillus metI gene, yields increased levels of methionine production. In exemplary microorganisms, endogenous enzymes subject to methionine feedback inhibition are complemented, added to, or circumvented by introduction of a methionine feedback resistant enzyme, thereby yielding increased methionine production. In certain embodiments of the present invention, microorganisms are utilized which have a diminished or ablated transsulfuration-based methionine biosynthetic pathway. These organisms may produce methionine only through the direct sulfhydrylation pathway and hence are particularly suited for increased production of methionine using exogenously introduced Bacillus met I.

In some embodiments, this invention relates to recombinant microorganisms lacking or having repressed MetB or MetC, where such a microorganism is deregulated for MetI. In some embodiments, recombinant microorganisms deregulated for metI lack MetB or include repressed MetB.

The metI in case of some recombinant microorganisms encompassed by this disclosure is a Bacillus MetI, such as for example, Bacillus subtilis MetI.

In some embodiments, recombinant microorganisms of the present invention belong to the genus Corynebacterium, such as, for example, Corynebacterium glutamicum.

Deregulation of metI can be achieved by one or more methods described herein and those known in the art. In some embodiments, deregulation of metI is achieved by overexpression of the metI gene.

Also encompassed by this invention are expression cassettes, for example, a MetI expression cassette, comprising the metI gene operatively linked to a heterologous promoter and, optionally a ribosomal binding site.

In some embodiments, a promoter used in a metI cassette is a P15 promoter.

Also encompassed by this invention are vectors for overexpression of metI. In some embodiments, a vector comprises a Met I expression cassette, as described herein.

In some embodiments, recombinant microorganisms described herein include a MetI expression cassette. In some embodiments, microorganisms are repressed for MetB and MetC in addition to including a metI expression cassette.

This invention further relates to a method for producing methionine, by culturing a recombinant microorganism which is repressed for or is lacking MetB and MetC and is deregulated for MetI, under conditions such that methionine is produced. A further step of isolating the methionine may be included in a method for producing methionine.

In some embodiments, methods for increasing methionine production capacity in a methionine-producing microorganism are described herein where such methods include deregulating metI in the microorganism, thereby to increase methionine production capacity of the microorganism.

In some embodiments, a method for increasing methionine production capacity in a microorganism exhibiting methionine feedback inhibition is described, where such method includes deregulating metI to alleviate methionine feedback inhibition, thereby increasing methionine production capacity of the microorganism.

In some embodiments, methionine production capacity is increased by at least 20% relative to a control microorganism.

In yet other embodiments, methionine production capacity is increased by at least 30% relative to a control microorganism.

Further, in some embodiments, methionine production capacity is increased by at least 40% relative to a control microorganism.

Also encompassed are recombinant microorganisms that have an increased capacity for methionine production, however, do not include deregulated MetI.

In another embodiment, installation of a heterologous metI gene in a microorganism is done in such a manner that the resulting engineered microorganism produces a second useful compound, for example a carotenoid compound, such as lycopene or astaxanthin, as a byproduct, such that two useful compounds can be co-produced. In another embodiment, an organism is engineered to co-produce a first compound such as an amino acid (for example, including but not limited to, methionine, lysine, glutamic acid, threonine, isoleucine, phenylalanine, tyrosine, tryptophan, alanine, cysteine, leucine, homoserine, homocysteine, etc.) or other a non-carotenoid compound of commercial interest (for example, including but not limited to, methane, hydrogen, lactic acid, 1,2-propane diol, 1,3-propane diol, ethanol, methanol, propanol, acetone, butanol, acetic acid, propionic acid, citric acid, itaconic acid, glucosamine, glycerol, sugars, vitamins, therapeutic, research and industrial enzymes, therapeutic, research and industrial proteins, and various salts of any of the above listed compounds) and a second compound including a carotenoid compound of commercial interest (for example, including but not limited to, lycopene, astaxanthin, β-carotene, lutein, zeaxanthin, canthaxanthin, decaprenoxanthin, and bixin, etc.). In a preferred embodiment, the first compound is separated as a gas or is secreted into a culture medium while the second, carotenoid compound, remains with the cell mass.

The present invention further relates to improved genetic engineering techniques, i.e. vector constructs, which facilitate the transfer of nucleic acid sequences into target microorganisms. One aspect of the improved methods and materials herein is novel recombinant expression vectors capable of transforming cells and thereby causing the expression of desired nucleic acid sequences. Preferably, these nucleic acid sequences comprise genes that facilitate or improve biosynthetic pathways of the target microorganism such that production of a desired substance is achieved, modified or increased. Such genes may encode enzymes or proteins involved in biosynthesis of e.g. sulfur-containing fine chemicals such as methionine. In preferred embodiments of the present invention the enzyme is an o-acetylhomoserine sulfhydrylase, o-succinylhomoserine sulfhydrylase or similar enzyme involved in the biosynthetic production of methionine.

In certain embodiments of the present invention, the recombinant expression vectors comprise integration cassettes. The recombinant expression cassettes are useful for the integration of nucleic acid sequences into specific, desired genomic regions of a target organism. In certain embodiments of the present invention, recombinant expression vectors comprising integration cassettes have been designed such that specific gene sequences are disrupted by the integration cassette and heterologous nucleic acid sequences inserted. These heterologous sequences may encode desired proteins or enzymes (e.g., methionine biosynthetic enzymes)

Also embodied herein are improved methods and materials useful for efficient screening of recombinant organisms comprising desired traits. In certain embodiments the screening is calorimetric screening. In preferred embodiments, the colorimetric screening is achieved by modifying levels of production of carotenoid compounds, such as, for example, lycopene, astaxanthin, β-carotene, lutein, zeaxanthin, canthaxanthin, decaprenoxanthin, and bixin, and the like in target cells. Accordingly the present invention provides material and methods for recombinantly modifying the carotenoid biosynthesis operon and thereby yielding genetically engineered transformants which may be selected based on phenotypic changes related to carotenoid production (e.g., color change).

The present invention further relates to novel expression vector designs for introducing nucleic acid sequences optionally comprising gene sequences into microorganisms

Compositions produced according to the above-described methodologies are also featured as are microorganisms utilized in said methodologies.

### BRIEF DESCRIPTION OF THE DRAWINGS

FIG. 1. provides a graphic illustration of methionine biosynthetic pathway utilized in the microorganisms of the invention

FIG. 2. is a graphic representation of experimental data derived from Example 2 showing the relative sensitivities of C. glutamicum Met Y and B. subtilis Met I to methionine inhibition.

FIG. 3. is a schematic representation the pOM284 plasmid for integration of a cassette comprising the metI gene.

FIG. 4. is a schematic representation of the carotenoid biosynthesis operon present in Corynebacterium glutamicum.

FIG. 5. is a schematic representation the pOM246 plasmid for integration of a cassette comprising the metI gene.

FIG. 6. is a schematic representation of carotenoid biosynthetic pathway of C. glutamicum.

FIG. 7A-C depicts a multiple sequence alignment (MSA) of the Bacillus subtilis Met I amino acid sequence set forth in SEQ ID NO:2 to fifty closest sequences found in NCBI's GENBANK® database. SEQ ID NOs:26-75 correspond to the amino acid sequences of Bacillus subtilis hypothetical protein (GENBANK®Accession No. NP_{—}389069.1) (SEQ ID NO:26), Bacillus licheniformis Cys/Met metabolism pyridoxal-phosphate-dependent enzyme (GENBANK® Accession No. AAU22849.1) (SEQ ID NO:27), Bacillus licheniformis clone ATCC 14580 (GENBANK® Accession No. YP_{—}090888.1) (SEQ ID NO:28) Geobacillus kaustophilus cystathionine gamma-synthase (GENBANK® Accession No YP_{—}146719.1) (SEQ ID NO:29), Bacillus halodurans cystathionine gamma-synthase (GENBANK® Accession No. BAB05346.1) (SEQ ID NO:30), Bacillus cereus cystathionine beta-lyase (GENBANK® Accession No. YP_{—}085587.1) (SEQ ID NO:31), Bacillus cereus cystathionine gamma-synthase (GENBANK® Accession No. ZP_{—}00238525.1) (SEQ ID NO:32), Bacillus thuringiensis cystathionine beta-lyase (GENBANK® Accession No. YP_{—}038316.1) (SEQ ID NO:33), Bacillus anthracis cystathionine beta-lyase (GENBANK® Accession No. YP_{—}021123.1) (SEQ ID NO:34), Bacillus cereus cystathionine beta-lyase ATCC 10987 (GENBANK® Accession No. NP_{—}980629.1) (SEQ ID NO:35), Bacillus cereus cystathionine gamma-synthase ATCC 14579 (GENBANK® Accession No. NP_{—}833967.1) (SEQ ID NO:36), Pasteurella mitocida subspecies (GENBANK® Accession No. NP_{—}245932.1) (SEQ ID NO:37), Hemophilus somnus COGO626 cystathioine beta-lyase/cystathionine gamma-synthase (GENBANK® Accession No. ZP_{—}00132603.1) (SEQ ID NO:38), Manheimia succiniciproducens MetC protein (GENBANK® Accession No. YP_{—}088819.1) (SEQ ID NO:39), Hemophilus somnus OGO626 cystathioine beta-lyase/cystathionine gamma-synthase (GENBANK® Accession No. ZP_{—}00122714.1) (SEQ ID NO:40), Hemophilus influenzae cystathionine gamma-synthase (GENBANK® Accession No. NP_{—}438259.1) (SEQ ID NO:41), cystathionine gamma synthase (GENBANK® Accession No. P44502) (SEQ ID NO:42), Hemophilus influenzae COGO626 cystathionine beta-lyase/cystathionine gamma-synthase (GENBANK® Accession No. ZP_{—}00322320.1) (SEQ ID NO:43), Hemophilus influenzae COGO626 cystathionine beta-lyase/cystathionine gamma-synthase (GENBANK® Accession No. ZP_{—}00157594.2) (SEQ ID NO:44), Hemophilus influenzae COGO626 cystathionine beta-lyase/cystathionine gamma-synthase (GENBANK® Accession No. ZP_{—}00154815.2) (SEQ ID NO:45), Bacillus clausii cystathionine gamma-synthase (GENBANK® Accession No. YP_{—}175363.1) (SEQ ID NO:46), Actinobacillus pleuropneumoniae COGO626 cystathionine beta-lyase/cystathionine gamma-synthase (GENBANK® Accession No. ZP_{—}00134030.2) (SEQ ID NO:47), Listeria monocytogenes cystathionine beta/gamma-lyase (GENBANK® Accession No. YP_{—}014300.1) (SEQ ID NO:48), Listeria monocytogenes cystathionine beta/gamma-lyase (GENBANK® Accession No. ZP_{—}00234337.1) (SEQ ID NO:49), Listeria monocytogenes hypothetical protein 1mo1680 (GENBANK® Accession No. NP_{—}465205.1) (SEQ ID NO:50), Listeria iinocua hypothetica protein lin1788 (GENBANK® Accession No. NP_{—}471124.1) (SEQ ID NO:51), Clostridium acetobutylicum cystathionine gamma-synthase (GENBANK® Accession No. NP_{—}347010.1) (SEQ ID NO:52), Symbiobacterium thermophilium cystathionine gamma-synthase (GENBANK® Accession No. YP_{—}076192.1) (SEQ ID NO:53), Lactobacillus plantarum O-succinylhomoserine (thiol)-lyase (GENBANK® Accession No. NP_{—}786043.1) (SEQ ID NO:54), Staphylococcus epidermis trans-sulfuration enzyme family protein (GENBANK® Accession No. YP_{—}187637.1) (SEQ ID NO:55), Staphylococcus epidermis ATCC 12228 (GENBANK® Accession No. NP_{—}765934.1) (SEQ ID NO:56), Clostridium thermocellum COGO0626 cystathionine beta-lyase/cystathionine gamma-synthase (GENBANK® Accession No. ZP_{—}00313823.1) (SEQ ID NO:57), Moorella thermoacetica COGO0626 cystathionine beta-lyase/cystathionine gamma-synthase (GENBANK® Accession No. ZP_{—}0030849.1) (SEQ ID NO:58), Streptococcus thermophilus cystathionine gamma-synthase (GENBANK® Accession No. YP_{—}140770.1) (SEQ ID NO:59), Streptococcus pneumoniae cystathionine gamma-synthase (GENBANK® Accession No. NP_{—}358970.1) (SEQ ID NO:60), Geobacter sulfurreducens cystathionine beta-lyase (GENBANK® Accession No. NP_{—}951998.1) (SEQ ID NO:61), Geobacter metallireducens COGO0626 cystathionine beta-lyase/cystathionine gamma-synthase (GENBANK® Accession No. ZP_{—}00298719.1) (SEQ ID NO:62), Streptococcus pneumonia transsulfuration enzyme family protein (GENBANK® Accession No. NP_{—}345975.1) (SEQ ID NO:63), Streptococcus anginosus cystathionine gamma-synthase (GENBANK® Accession No. BAC41490.1) (SEQ ID NO:64), Streptacoccus mutans putative cystathionine gamma-synthase (GENBANK® Accession No. AAN59314.1) (SEQ ID NO:65), Bacillus lichenformis cystathionine gamma-lyase (GENBANK® Accession No. AAU24359.1) (SEQ ID NO:66), Lactococcus lactis cystathionine gamma-synthase (GENBANK® Accession No. NP_{—}268074.1) (SEQ ID NO:67), Staphylococcus aureus Cys/Met metabolism PLP-dependent enzyme (GENBANK® Accession No. CAG42106.1) (SEQ ID NO:68), Staphylococcus aureus trans-sulfuration enzyme family protein (GENBANK® Accession No. YP_{—}185322.1) (SEQ ID NO:69), Staphylococcus aureus Cys/met metabolism PLP-dependent enzyme (GENBANK® Accession No. CAG39379.1) (SEQ ID NO:70), Helicobacter hepaticus cystathionine gamma-synthase (GENBANK® Accession No. AAP76659.1) (SEQ ID NO: 71), Enterococcus faecium COGO0626 cystathionine beta-lyase/cystathionine gamma-synthase (GENBANK® Accession No. ZP_{—}00285445.1) (SEQ ID NO:72), Anabaena variabilis COGO0626 cystathionine beta-lyase/cystathionine gamma-synthase (GENBANK® Accession No. ZP_{—}00351535.1) (SEQ ID NO:73), Streptococcus suis COGO0626 cystathionine beta-lyase/cystathionine gamma-synthase (GENBANK® Accession No. ZP_{—}00332320.1) (SEQ ID NO:74), and Lactococcus lactis cystathionine gamma synthase (GENBANK® Accession No. NP_{—}266937.1) (SEQ ID NO:75). The alignment was generated using ClustalW MSA software at the GenomeNet CLUSTALW Server at the Institute for Chemical Research, Kyoto University. The following parameters were used: Pairwise Alignment, K-tuple (word) size=1, Window size=5, Gap Penalty=3, Number of Top Diagonals=5, Scoring Method=Percent; Multiple Alignment, Gap Open Penalty=10, Gap Extension Penalty=0.0, Weight Transition=No, Hydrophilic residues=Gly, Pro, Ser, Asn, Asp, Gln, Glu, Arg and Lys, Hydrophobic Gaps=Yes; and Scoring Matrix=BLOSUM.

### DETAILED DESCRIPTION OF THE INVENTION

The present invention is based, at least in part, on the discovery that certain Bacillus genes/enzymes involved in the biosynthesis of methionine are not subject to methionine feedback inhibition. These genes, when utilized in heterologous microorganisms, enhance the endogenous methionine biosynthetic pathway, thus providing recombinant microorganisms capable of increased methionine output.

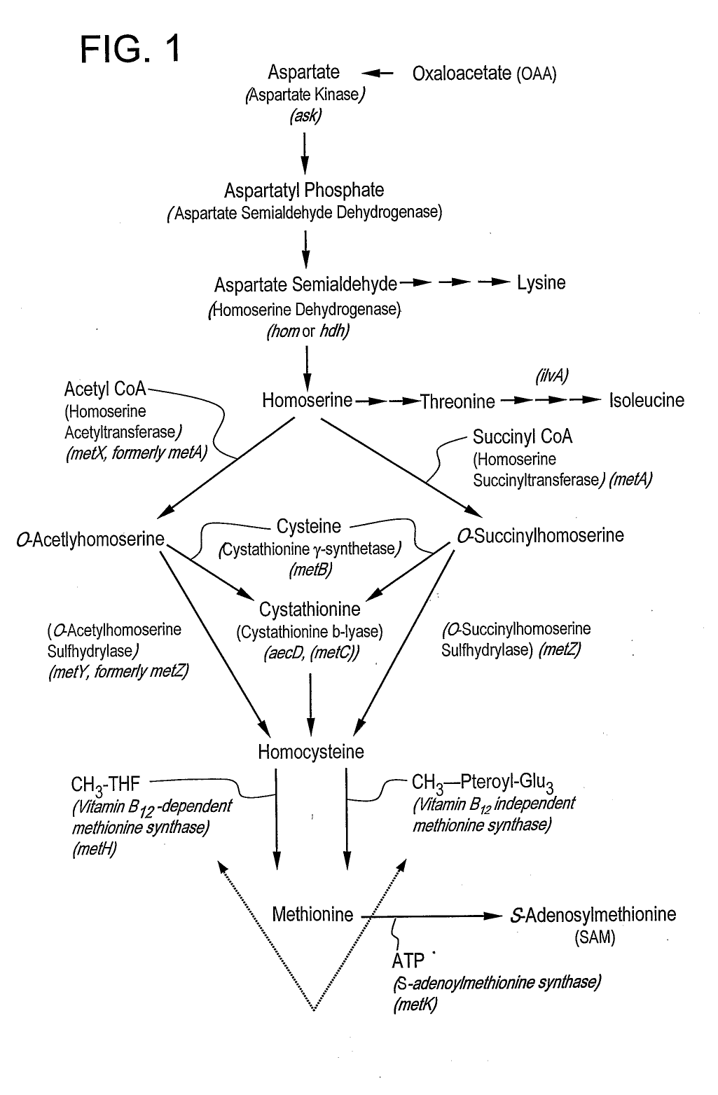

Two alternative pathways exist for the addition of sulfur atoms to precursor substrates in methionine synthesis in microorganisms (see FIG. 1). E. coli, e.g., utilizes a transsulfuration pathway, whereas, other microorganisms such as Saccharomyces cerevisiae and Corynebacterium glutamicum have, in addition, developed a direct sulfhydrylation pathway. Although many microorganisms use either transsulfuration or direct sulfhydrylation, but not both, C. glutamicum employs both pathways for synthesis of methionine.

The transsulfuration and direct sulfhydrylation pathways both begin with either O-acetyl-homoserine or O-succinyl-homoserine, and result in the intermediate homocysteine, a precursor to methionine. In the transsulfuration pathway, cysteine is the sulfur donor contributing to formation of cystathionine, a reaction catalyzed by the enzyme MetB (cystathionine-gamma-synthase). Cystathionine is subsequently cleaved to homocysteine and pyruvate, in a reaction catalyzed by MetC (cystathione-beta-lyase). In the direct sulfhydrylation pathway utilizing O-acetyl-homoserine, MetY (O-acetylhomoserine sulfhydrylase) catalyzes. the direct addition of sulfide to O-acetyl-homoserine to form homocysteine. Production of homocysteine directly from O-succinyl-homoserine is similarly accomplished by MetZ (O-succinyl-homoserine sulfhydrylase). In some of the prior art, the terms MetY and MetZ are used interchangeably, in part because MetY is known to be active on O-succinyl-homoserine in addition to its normal substrate, O-acetyl-homoserine (Hwang et al., (2002) J. Bacteriol. 184:1277-1286).

A number of experiments performed by the present inventors have indicated that MetY activity is a rate limiting step in methionine biosynthesis in strains of Corynebacterium engineered to favor the direct sulfhydrylation pathway (with a repressed metB), for example, the related M2014 and OM99 (McbR⁺) strain backgrounds. In particular, O-acetyl-homoserine, one of the substrates for MetY, builds up to relatively high levels in strains containing the replicating plasmid H357, which expresses metA (sometimes referred to as metY) and metY. Further, it is known from enzyme assays that MetY is sensitive to feedback inhibition by methionine. A recent publication (Auger et al., 2002 Microbiology 148: 507-518) characterizes the Bacillus subtilis gene, metI, which encodes an O-acetyl-homoserine sulfhydrylase that carries out the same function as C. glutamicum MetY. Interestingly, the metI enzyme also has substantial MetB-like activity, cystathionine-gamma-synthase (see Table 1). Furthermore, the B. subtilis genome contains no MetB homolog other than MetI. It is thus presumed that metI performs the functions of both MetY and MetB in its native host. This hypothesis is supported by the fact that the B. subtilis metI complements an E. coli metB⁻ auxotroph. In most, if not all, other microorganisms that have been studied to date, MetY-like activity is feedback inhibited by methionine, while MetB activity is not. Thus, it may be inferred that Bacillus metI evolved to be resistant to methionine inhibition in order to function efficiently in the MetB-like pathway.

TABLE 1
Reported specific activities of MetZ, MetB, and MetI.

| | O—Ac-Hse | | Cystathionine | | |
|---|---|---|---|---|---|
| | sulfhydrylase | | γ-synthase | | Inhibition by |
| Enzyme (reference) | activity | | activity | | methionine |
| Cg1 MetY (H.-S. Lee, | 6.0 | μMole/min · mg | Not determined | | Yes |
| personal | | | | | |
| communication) | | | | | |
| Cg1 MetB (H.-S. Lee, | 1.4 | μMole/min · mg | 7.4 | μMole/min · mg | No |
| personal | | | | | |
| communication) | | | | | |
| Bsu MetI (Auger et al., | 7.0 | μMole/min · mg | 1.5 | μMole/min · mg | ? |
| from T7 promoter in E. | | | | | |
| coli) | | | | | |
| Bsu MetI in | 199 | nMole/min | 1.9 | μMole/min | No |
| E. coli from P₁₅, | | | | | |
| crude extracts | | | | | |
| Bsu MetI in | 6.3 | nMole/min | 30.2 | nMole/min | No |
| C. glutamicum from | | | | | |
| P₁₅, | | | | | |
| crude extracts | | | | | |

The present invention provides recombinant microorganisms that have been genetically engineered to express a heterologous methionine biosynthetic enzyme.

In addition, the present invention provides for recombinant expression vectors useful for inserting heterologous nucleic acid sequences in the carotenoid operon of, e.g., Corynebacterium. These recombinant vectors may further comprise integration cassettes that target specific nucleic acid sequences of the carotenoid operon, e.g., protein coding or expression regulatory sequences. Further, these vectors and integration cassettes may be used to modify the operon such that production of carotenoids in the target organism results in phenotypic alteration, e.g. pigmentation change of the organism and alteration of the carotenoid(s) produced. This allows coproduction of a desirable carotenoid together with a desired amino acid, such as, for ex ample, methionine, lysine, glutamic acid, threonine, isoleucine, phenylalanine, tyrosine, tryptophan, alanine, leucine, cysteine, and the like.

In order that the present invention may be more readily understood, certain terms are first defined herein.

The phrase “biosynthetic pathway” or “biosynthetic process” is used herein to mean an in vivo or in vitro process whereby a molecule or compound of interest is produced as the result of one or several biochemical reactions. Generally, beginning with a precursor molecule, a prototypical biosynthetic process involves the action of one or several enzymes functioning in a stepwise fashion to produce a molecule or compound of interest. The end-product is usually a carbon containing molecule. Molecules or compounds of interest comprise e.g. small organic molecules, amino acids, peptides, cellular cofactors, vitamins, nucleotides, and similar chemical entities. Molecules or compounds of interest further comprise fine sulfur containing chemicals such as methionine, homocysteine, S-adenosylmethionine, glutathione, cysteine, biotin, thiamine, mycothiols, coenzyme A, coenzyme M, and lipoic acid. In certain circumstances, an enzyme or enzymes functioning in a biosynthetic pathway may be regulated by chemical products generated in the process. In such cases, a feedback loop is said to exist wherein increasing concentrations of an end or intermediate product modify the level, functioning, or activity of enzymes within the pathway. For example, the ultimate product of a biosynthetic process may act to down-regulate the activity of an enzyme in the biosynthetic process and thereby decrease the rate at which a desired end product is produced. Situations such as this are often undesirable in e.g. large scale fermentative processes used in industry for the production of molecules or compounds of interest. The methods and materials of the present invention are directed, at least in part, to improving industrial scale, fermentative production of compounds of interest. A typical example of a feedback loop occurs in the production of methionine described infra.

The term “methionine biosynthetic pathway” includes the biosynthetic pathway involving methionine biosynthetic enzymes (e.g., polypeptides encoded by biosynthetic enzyme-encoding genes), compounds (e.g., precursors, substrates, intermediates or products), cofactors and the like utilized in the formation or synthesis of methionine. The term “methionine biosynthetic pathway” includes the biosynthetic pathway leading to the synthesis of methionine in a microorganisms (e.g., in vivo) as well as the biosynthetic pathway leading to the synthesis of methionine in vitro. FIG. 1 depicts a schematic representation of the methionine biosynthetic pathway. As outlined in FIG. 1, synthesis of methionine from oxaloacetate (OAA) proceeds via the intermediates, aspartate, aspartate (aspartyl) phosphate and aspartate semialdehyde. Aspartate semialdehyde is converted to homoserine by homoserine dehydrogenase (the product of the hom gene, also known as thrA, metL, hdh, hsd, among other names in other organisms). The subsequent steps in methionine synthesis can proceed through the transsulfuration pathway and/or the direct sulfhydrylation pathway.

The term “methionine biosynthetic enzyme” includes any enzyme utilized in the formation of a compound (e.g., intermediate or product) of the methionine biosynthetic pathway. “Methionine biosynthetic enzyme” includes enzymes involved in e.g., the “transsulfuration pathway” and in the “direct sulfhydrylation pathway”, alternative pathways for the synthesis of methionine. For example, E. coli, utilizes a transsulfuration pathway, whereas, other microorganisms such as Saccharomyces cerevisiae have developed a direct sulfhydrylation pathway.

“Methionine biosynthetic enzymes” encompass all enzymes normally found in microorganisms that contribute to the production of methionine. They include enzymes involved in, for example, the transsulfuration pathway wherein homocysteine is formed from cysteine and O-acetyl-homoserine or cysteine and O-succinyl-homoserine. In the transsulfuration pathway, homoserine is converted to either O-acetyl-homoserine by homoserine acetyltransferase (the product of the metX gene) and the addition of acetyl CoA, or to O-succinyl-homoserine by the addition of succinyl CoA and the product of the metA gene (homoserine succinyltransferase). Donation of a sulfur group from cysteine to either O-acetyl-homoserine or O-succinyl-homoserine by cystathionine-gamma-synthase, the product of the metB gene, produces cystathionine. Cystathionine is then converted to homocysteine by cystathionine beta-lyase, the product of the metC gene (also referred to as the aecD gene in some organisms). Methionine biosynthetic enzymes also comprise enzymes in the direct sulfhydrylation pathway wherein an enzyme with O-acetyl-homoserine sulfhydralase (e.g. the mety gene of Corynebacterium—sometimes also referred to as the metZ gene) activity converts O-acetyl-homoserine to homocysteine in a single step process utilizing sulfide as a source of sulfur atoms. Homocysteine can also be formed in the direct sulfhydrylation pathway by the direct addition of sulfide to O-succinyl-homoserine by O-succinyl-homoserine sulfhydrylase, the product of the metZ gene.

Regardless of which pathway is used, the transsulfuration pathway or the direct sulfhydrylation pathway, methionine is subsequently produced from homocysteine by the addition of a methyl group by vitamin B₁₂-dependent methionine synthase (the product of the metH gene) or vitamin B₁₂-independent methionine synthase (the product of the metE gene).

The present invention is directed, in part, to the enzymes involved in the production of methionine (methionine biosynthetic enzymes) in gram positive bacteria as embodied in the genera Bacillus and Corynebacterium. Exemplary methionine biosynthetic enzymes present in microorganisms are provided in FIG. 1. These enzymes include e.g. aspartate kinase, aspartate semialdehyde dehydrogenase, homoserine dehydrogenase, homoserine acetyltransferase (present e.g. in Bacillus subtilis and C. glutamicum), homoserine succinyltransferase (present e.g. in Escherichia coli), O-acetyl-homoserine sulfhydralase, O-succinyl-homoserine sulfhydrylase, cystathionine γ-synthases, cystathionine β-lyase, methylene tetrahydrofolate reductase, vitamin B12-dependent methionine synthase and cobalamin-independent methionine synthase.

As described herein, a “MetI” enzyme has: (1) both O-acetyl-homoserine sulfhydrylase activity (also known as O-acetyl-homoserine sulfhydrolase; O-acetyl-homoserine thiolyase) and cystathionine-gamma-synthase activity, and optionally also have activity as an O-succinyl-homoserine sulfhydrylase (also known as O-succinyl-homoserine sulfhydrolase; O-succinyl-homoserine thiolyase) and a cystathionine-gamma-synthase; (2) has at least about 65% sequence identity to the Bacillus subtilis MetI amino acid sequence set forth as SEQ ID NO:2 comprising an O-acetyl-homoserine sulfhydrylase or an O-succinyl-homoserine sulfhydrylase that is substantially resistant to inhibition by methionine.

The term “manipulated microorganism” includes a microorganism that has been engineered (e.g., genetically engineered) or modified such that the microorganism has at least one enzyme of the methionine biosynthetic pathway modified in amount or structure such that methionine production is increased. Modification or engineering of such microorganisms can be according to any methodology described herein including, but not limited to, deregulation of a biosynthetic pathway and/or overexpression of at least one biosynthetic enzyme. A “manipulated” enzyme (e.g., a “manipulated” biosynthetic enzyme) includes an enzyme, the expression, production, or activity of which has been altered or modified such that at least one upstream or downstream precursor, substrate or product of the enzyme is altered or modified (e.g., an altered or modified level, ratio, etc. of precursor, substrate and/or product), for example, as compared to a corresponding wild-type or naturally occurring enzyme. A “manipulated” enzyme also includes one where resistance to inhibition, e.g., feedback inhibition, by one or more products or intermediates has been enhanced. For example, an enzyme that is capable of enzymatically functioning efficiently in the presence of, e.g., methionine.

In some embodiments, genes encompassed by this invention are derived from Bacillus. The term “derived from Bacillus” or “Bacillus-derived” includes a gene which is naturally found in microorganisms of the genus Bacillus. In some embodiments, genes of the present invention are derived from a microorganism selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus anthracis, Bacillus halodurans, and other Group 1 Bacillus species, for example, as characterized by 16S rRNA type. In yet other embodiments, a gene is derived from Bacillus brevis or Bacillus stearothermophilus. In some embodiments, genes of the present invention are derived from a microorganism selected from the group consisting of Bacillus licheniformis, Bacillus amyloliquefaciens, Bacillus subtilis, and Bacillus pumilus. In some embodiments, the gene is derived from Bacillus subtilis (e.g., is Bacillus subtilis-derived). The terms “derived from Bacillus subtilis” and “Bacillus subtilis derived,” are used interchangeably herein and include a gene which is naturally found in the microorganism Bacillus subtilis. Included within the scope of the present invention are Bacillus-derived genes (e.g., B. subtilis-derived genes), for example, Bacillus or B. subtilis metI genes.

The term “gene,” as used herein, includes a nucleic acid molecule (e.g. a DNA molecule or segment thereof) that, in an organism, can be separated from another gene or other genes, by intergenic DNA (i.e., intervening or spacer DNA which naturally flanks the gene and/or separates genes in the chromosomal DNA of the organism). Alternatively, a gene may slightly overlap another gene (e.g., the 3′ end of a first gene overlapping the 5′ end of a second gene), the overlapping genes separated from other genes by intergenic DNA. A gene may direct synthesis of an enzyme or other protein molecule (e.g., may comprise coding sequences, for example, a contiguous open reading frame (ORF) which encodes a protein) or may itself be functional in the organism. A gene in an organism, may be clustered in an operon, as defined herein, said operon being separated from other genes and/or operons by the intergenic DNA. An “isolated gene,” as used herein, includes a gene which is essentially free of sequences which naturally flank the gene in the chromosomal DNA of the organism from which the gene is derived (i.e., is free of adjacent coding sequences that encode a second or distinct protein, adjacent structural sequences or the like) and optionally includes 5′ and 3′ regulatory sequences, for example promoter sequences and/or terminator sequences. In one embodiment, an isolated gene includes predominantly coding sequences for a protein (e.g., sequences which encode Bacillus proteins). In another embodiment, an isolated gene includes coding sequences for a protein (e.g., for a Bacillus protein) and adjacent 5′ and/or 3′ regulatory sequences from the chromosomal DNA of the organism from which the gene is derived (e.g., adjacent 5′ and/or 3′ Bacillus regulatory sequences). Preferably, an isolated gene contains less than about 10 kb, 5 kb, 2 kb, 1 kb, 0.5 kb, 0.2 kb, 0.1 kb, 50 bp, 25 bp or 10 bp of nucleotide sequences which naturally flank the gene in the chromosomal DNA of the organism from which the gene is derived.

The term “operon” includes at least two adjacent genes or ORFs, optionally overlapping in sequence at either the 5′ or 3′ end of at least one gene or ORF. The term “operon” includes a coordinated unit of gene expression that contains a promoter and possibly a regulatory element associated with one or more adjacent genes or ORFs (e.g., structural genes encoding enzymes, for example, biosynthetic enzymes). Expression of the genes can be coordinately regulated, for example, by regulatory proteins binding to the regulatory element or by anti-termination of transcription. The genes of an operon (e.g., structural genes) can be transcribed to give a single mRNA that encodes all of the proteins.

Various aspects of the invention are described in further detail in the following subsections.

### I. Methods and Microorganisms for the Increased Production of Methionine in Heterologous Microorganisms

C. glutamicum harbors two pathways for methionine synthesis, the direct sulfhydrylation pathway and the transulfuration pathway. (see FIG. 1). The pathways utilize O-acetyl-homoserine and yields homocysteine, a precursor to methionine. In the transulfuration pathway, O-acetyl-homoserine is converted to cystathione by MetB in the presence of cysteine. Cystathionine is subsequently cleaved to homocysteine and pyruvate, in a reaction catalyzed by MetC. In the direct sulfhydrylation pathway MetY catalyzes the direct addition of sulfide to O-acetyl-homoserine to form homocysteine. As described supra, Met Y activity is believed to be a rate limiting step in microorganisms that utilize the direct sulfhydrylation pathway. Table 1 depicts various enzymes in the methionine biosynthetic pathway.

TABLE II
Enzymes in the methionine biosynthetic
pathway and the genes encoding them

| Enzyme | Gene |
|---|---|
| Aspartate kinase | ask |
| Homoserine Dehydrogenase | hom |
| Homoserine Acetyltransferase | metX |
| Homoserine Succinyltransferase | metA |
| Cystathionine γ-synthetase | metB |
| Cystathionine β-lyase | metC |
| O-Acetylhomoserine sulfhydrylase | metY |
| O-Succinylhomoserine sulfhydrylase | metZ |
| Vitamin B₁₂-dependent methionine synthase | metH |
| Vitamin B₁₂-independent methionine synthase | metE |
| N^{5,10}-methylene-tetrahydrofolate reductase | metF |
| S-adenosylmethionine synthase | metK |

The present invention features the modification of microorganisms, for example, through the use of genetic engineering such that the modified microorganisms are capable of increased production of methionine. More specifically, in some embodiments, genetic engineering methods involve introduction of a heterologous gene or genes encoding enzymes that function within endogenous biosynthetic pathways such that the production of methionine is modified or increased. Preferably, the enzyme is resistant to methionine feedback inhibition. The phrase “resistant to methionine feedback inhibition,” as used herein, refers to an enzyme that is capable of functioning enzymatically with a significant activity in the presence of methionine. An enzyme that is resistant to methionine feedback inhibition may function significantly in the presence of, for example, 1-10 μM, 10-100 μM or 100 μM-1 mM methionine. In some embodiments of the present invention, an enzyme of interest is capable of functioning at concentrations of 1-10 mM, 10-100 mM or even higher concentrations of methionine. The present invention particularly encompasses methionine feedback resistant enzymes that are involved in the biosynthetic pathways or processes that result in the production of methionine.

The present invention features methods of producing increased levels of methionine from microorganisms. As used herein, the phrase “increased level of methionine production” refers to a level or amount of methionine greater (e.g. 5% greater, 10% greater, 15% greater, 20% greater, 30% greater, 40% greater, or more) than that produced by an unmodified microorganism or other suitable control microorganism. In exemplary embodiments, the level of methionine production is at least 50%, 60% or 70% greater than that produced by an unmodified microorganism or other suitable control microorganism. In yet other embodiments, the level of production is at least about 100% greater (i.e. 2-fold, 3-fold, 4-fold 5-fold or even 10-fold greater, or more) than that produced by an unmodified microorganism or other suitable control microorganism. Values and ranges included in and/or intermediate of the values set forth herein are also intended to be encompassed by the invention. In exemplary embodiments, increased levels of methionine production are also intended to encompass amounts produced above a basal level established by microorganisms that have not been genetically engineered to express a heterologous methionine resistant biosynthetic enzyme.

Accordingly, the present invention provides a method of producing methionine, comprising culturing a “methionine-producing microorganism”. A “methionine-producing microorganism” is any microorganism capable of producing methionine, e.g., bacteria, yeast, fungus, Archaea, etc. In one embodiment, the methionine producing microorganism belongs to the genus Corynebacterium or Brevibacterium. In another embodiment, the methionine producing microorganism is Corynebacterium glutanicum. In yet another embodiment, the methionine producing microorganism is selected from the group consisting of: Escherichia coli or related Enterobacteria, Bacillus subtilis or related Bacillus, Saccharomyces cerevisiae or related yeast strains

The present invention is based, at least in part, on the discovery that certain strains of C. glutamicum can be genetically engineered to express enzymes which are resistant to methionine feedback inhibition, bypassing and/or adding to endogenous methionine feedback sensitive enzymes, e.g., the product of the metY and/or the metZ gene. The heterologous genes introduced into microorganisms, include, for example, MetI, an enzyme having O-acetyl homoserine sulfhydrylase activity and cystathione-gamma synthase activity in vitro, or having O-succinyl homoserine sulfhydrylase activity and cystathione -gamma synthase activity, wherein the O-acetyl homoserine sulfhydrylase or O-succinyl homoserine sulfhydrylase activity is resistant to methionine feedback inhibition.

### II. Recombinant Microorganisms

The present invention features microorganisms for use in the production of fine chemicals. In one embodiment, a microorganism of the present invention is a Gram positive organism (e.g., a microorganism which retains basic dye, for example, crystal violet, due to the presence of a Gram-positive wall surrounding the microorganism). In some embodiments, the microorganism is a microorganism belonging to a genus selected from the group consisting of Bacillus, Brevibacterium, Cornyebacterium, Lactobacillus, Lactococci and Streptomyces. In yet other embodiments, the microorganism is of the genus Corynebacterium. Additionally, in some embodiments, the microorganism is selected from the group consisting of Corynebacterium glutamicum, Corynebacterium efficiens, Corynebacterium lilium, Corynebacterium diphtheriae, Corynebacterium pseudotuberculosis and Corynebacterium pyogenes.

Exemplary aspects of the invention feature recombinant microorganisms, in particular, recombinant microorganisms including vectors or genes (e.g., wild-type and/or mutated genes) as described herein. As used herein; the term “recombinant microorganism” includes a microorganism (e.g., bacteria, yeast cell, fungal cell etc.) that has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism from which it was derived. The genetic alterations described herein can be accomplished, for example, by in vitro manipulation of DNA sequences or by classical genetic methods of mating, transduction, transformation, etc.

In some embodiments, the microorganism is a Gram negative (excludes basic dye) organism. In other embodiments, the microorganism is a microorganism belonging to a genus selected from the group consisting of Salmonella, Escherichia, Klebsiella, Serratia, and Proteus. In yet other embodiments, the microorganism belongs to the genus Escherichia, for example, Escherichia coli. In some embodiments, the microorganism belongs to the genus Saccharomyces (e.g., S. cerevisiae).

In certain embodiments, a recombinant microorganism is modified or engineered such that at least one non-native methionine biosynthetic enzyme is expressed or overexpressed. The terms “overexpressed” and “overexpression” include expression of a gene product (e.g., a biosynthetic enzyme) constitutively or at a level greater than that expressed prior to modification or engineering of the microorganism or in a comparable microorganism that has not been manipulated. In some embodiments, the microorganism can be genetically designed or engineered to overexpress a level of gene product greater than that expressed in a comparable microorganism that has not been engineered.

In some embodiments, a microorganism can be physically or environmentally manipulated to overexpress a level of gene product greater than that expressed prior to manipulation of the microorganism or in a comparable microorganism which has not been manipulated. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

Genetic engineering can include, but is not limited to, altering or modifying regulatory sequences or sites associated with expression of a particular gene (e.g. by adding strong promoters, constitutive promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor or biosynthetic proteins and/or the use of mutator alleles, e.g., bacterial alleles that enhance genetic variability and accelerate, for example, adaptive mutation). Genetic engineering can also include deletion of a gene, for example, to block a pathway or to remove a repressor.

In certain embodiments, a microorganism of the invention is a “Campbell in” or “Campbell out” microorganism (or cell or transformant). As used herein, the phrase “Campbell in” transformant shall mean a transformant of an original host cell in which an entire circular double stranded DNA molecule (for example a plasmid) has integrated into a chromosome of the cell by a single homologous recombination event (a cross in event), and which effectively results in the insertion of a linearized version of the circular DNA molecule into a first DNA sequence of the chromosome that is homologous to a first DNA sequence of the circular DNA molecule. The phrase “Campbelled in” refers to the linearized DNA sequence that has been integrated into the chromosome of the “Campbell in” transformant. A “Campbell in” transformant contains a duplication of the first homologous DNA sequence, that includes and surrounds the homologous recombination crossover point.

“Campbell out” refers a cell descended from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of the linearized insert, the second recombination event resulting in the deletion (jettisoning) of a portion of the integrated DNA sequence, but, importantly, also resulting in a portion (this can be as little as a single base) of the integrated DNA sequence remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, and so on.

The homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and second homologous DNA sequence are usually separated by a region of partial non-homology, and it is this region of non-homology that remains deposited in the chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. A preferred length for the first and second homologous sequences is about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

### III. The MetI Gene and Homologs Thereof

In Bacillus subtilis, the metI and metC genes are located in the recently elucidated metIC operon (Auger et al., (2002) Microbiology 148:507-518). Formerly, the B. subtilis metI gene was designated as yjcI and metC designated as yjcJ. Transcription from the metIC operon in B. subtilis is regulated by the source of sulfur. When cysteine or sulfate is the sole sulfur source transcription is high, whereas, when the sole sulfur source is methionine its transcription is low.

By homology comparison of the protein sequences, the metI and MetC enzymes belong to the cystathionine gamma synthase family of proteins which includes cystathionine gamma-synthase, cystathionine beta-lyase, cystathionine gamma-lyase and O-acetylhomoserine sulfhydrylase. The family is distinguished by the amino acid motif [DQ]-[LIVMF]-X₃-[STAGC]-[STAGCI]-T-K-[FYWQ]-[L]-X-G-[HQ]-[SGNH] (SEQ ID NO: 76) which encompasses a lysine residue critical to binding of the common co-factor pyridoxal phosphate. The MetC enzyme has cystathionine beta-lyase activity, whereas, metI has both O-acetylhomoserine sulfhydrylase and cystathionine gamma synthase activity or O-succinylhomoserine sulfhydrylase and cystathionine gamma synthase activity.

The present invention pertains to enzymes having an O-acetylhomoserine sulfhydrylase activity and/or O-succinylhomoserine sulfhydrylase activity. The present invention also pertains to enzymes that have cystathione gamma synthetase activity. In certain embodiments, the invention comprises enzymes that have both O-acetylhomoserine sulfhydrylase activity and cystathione gamma synthetase activity. In other embodiments, the present invention encompasses enzymes which have O-succinyl homoserine sulfhydrylase activity. In yet other embodiments, the present invention comprises both O-succinyl homoserine sulfhydrylase and cystathione gamma synthetase activity.

The present invention encompasses enzymes having functional and structural homology to the metI enzyme of B. subtilis. By “functional homology” it is meant that e.g., the homologous enzyme has the capability of acting in an enzymatic fashion substantially similar to the metI enzyme i.e. as a methionine resistant mediator of the biochemical sulfhydrylation of O-acetylhomeserine to produce homocysteine or as a methionine resistant mediator of the biochemical sulfhydrylation of O-succinylhomoserine to produce homocysteine. In the sense used herein the terms “homology” and “homologous” are not limited to designate proteins having a theoretical common genetic ancestor, but includes proteins which may be genetically unrelated that have, none the less, evolved to perform similar functions and/or have similar structures. Functional homology to the metI enzyme of B. subtilis also encompasses enzymes that have the characteristic of acting as a cystathione gamma synthetase, wherein, cystathionine is produced from cysteine and O— succinylhomoserine or wherein cystathionine is produced from cysteine and O-acetylhomoserine. For proteins to have functional homology, it is not required that they have significant identity in their amino acid sequences, but, rather, proteins having functional homology are so defined by having similar or identical activities, e.g., enzymatic activities. Similarly, proteins with structural homology are defined as having primary (sequence) or analogous secondary, tertiary (or quaternary) structure, but do not necessarily require nucleic acid or amino acid identity. In certain circumstances, structural homologs may include proteins that maintain structural homology only at the active site or substrate binding site of the protein.

In addition to structural and functional homology, the present invention further encompasses proteins having at least partial nucleic acid or amino acid identity to the MetI enzyme of B. subtilis. To determine the percent of partial identity of two amino acid sequences or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence is occupied by the same amino acid residue or nucleotide as the corresponding position in the other, then the molecules are identical at that position. The percent identity between the two sequences is a function of the number of identical positions shared by the sequences (i.e., % identity y=# of identical positions/total # of positions multiplied by 100). Percent identity can also be determined by aligning two nucleotide sequences using the Basic Local Alignment Search Tool (BLAST™) program.

Accordingly, one aspect of the invention pertains to isolated nucleic acid molecules (e.g., cDNAs, DNAs, or RNAs) comprising a nucleotide sequence encoding a protein (or biologically active portions thereof) identical to the metI enzyme of B. subtilis. In some embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more identical to the nucleotide sequence of B. subtilis metI as set forth in SEQ ID NO: 1, or a portion thereof.

In some embodiments, the isolated nucleic acid molecule encodes a protein or portion thereof wherein the protein or portion thereof includes an amino acid sequence which is sufficiently similar or identical to the amino acid sequence of B. subtilis MetI such that the protein or portion thereof exhibits the activity of an O-acetylhomoserine sulfhydrylase and cystathionine gamma synthase or O-succinylhomoserine sulfhydrylase and cystathionine gamma synthase. Preferably, the protein or portion thereof encoded by the nucleic acid molecule is resistant or has reduced sensitivity to methionine feedback inhibition. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more identical to the amino acid sequence of B. subtilis metI as set forth in SEQ ID NO: 2, or a portion thereof.

The present invention also comprises techniques well known in the art useful for the genetic engineering of the proteins described herein to produce enzymes with improved or modified characteristics. For example, it is well within the teachings available in the art to modify a protein such that the protein has increased or decreased substrate binding affinity. It also may be advantageous, and within the teachings of the art, to design a protein that has increased or decreased enzymatic rates. Particularly for multifunctional enzymes, it may be useful to differentially fine tune the various activities of a protein to perform optimally under specified circumstances. Further the ability to modulate an enzyme's sensitivity to feedback inhibition (e.g. by methionine) may be accomplished through selective change of amino acids involved in coordination of methionine or other cofactors which may be involved in negative or positive feedback. Further, genetic engineering encompasses events associated with the regulation of expression at the levels of both transcription and translation. For example, when a complete or partial operon is used for cloning and expression, regulatory sequences e.g. promoter or enhancer sequences of the gene may be modified such that they yield desired levels of transcription. It has also been demonstrated that Bacillus contains transcriptional regulatory sequences, e.g., S-boxes, which are sensitive to down-stream products of the methionine biosynthetic pathway (e.g., S-adenosyl methionine). Similarly, these nucleic acid motifs may be modified to achieve desired levels of enzyme, e.g., metI expression.

### IV. Recombinant Nucleic Acid Molecules and Vectors

The present invention further features recombinant nucleic acid molecules (e.g., recombinant DNA molecules) that include genes described herein (e.g., isolated genes), preferably Bacillus genes, more preferably Bacillus subtilis genes, even more preferably Bacillus subtilis methionine biosynthetic genes. The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). Preferably, a recombinant nucleic acid molecule (e.g., a recombinant DNA molecule) includes an isolated gene of the present invention operably linked to regulatory sequences. The phrase “operably linked to regulatory sequence(s)” means that at least a portion (usually the protein coding portion plus or minus several base pairs, e.g., 2, 3, 4 or more base pairs) of the nucleotide sequence of the gene of interest is linked to the regulatory sequence(s) in a manner which allows for expression (e.g., enhanced, increased, constitutive, basal, attenuated, decreased or repressed expression) of the gene, preferably expression of a gene product encoded by the gene (e.g. when the recombinant nucleic acid molecule is included in a recombinant vector, as defined herein, and is introduced into a microorganism). The term “heterologous nucleic acid” is used herein to refer to nucleic acid sequences not typically present in a target organism. They may also comprise nucleic acid sequences already present in a wild type strain of a target organism, but not normally found in a particular genetic region of a target organism of interest. Similarly, the term “heterologous gene” refers to a gene or an arrangement of a gene not present in a wild type strain of a target organism. Heterologous nucleic acids and heterologous genes generally comprise recombinant nucleic acid molecules. The heterologous nucleic acid or heterologous gene may or may not comprise modifications (e.g., by addition, deletion or substitution of one or more nucleotides).

The term “regulatory sequence” includes nucleic acid sequences which affect (e.g., modulate or regulate) expression of other nucleic acid sequences (i.e., genes). In one embodiment, a regulatory sequence is included in a recombinant nucleic acid molecule in a similar or identical position and/or orientation relative to a particular gene of interest as is observed for the regulatory sequence and gene of interest as it appears in nature, e.g., in a native position and/or orientation. For example, a gene of interest can be included in a recombinant nucleic acid molecule operably linked to a regulatory sequence which accompanies or is adjacent to the gene of interest in the natural organism (e.g., operably linked to “native” regulatory sequences (e.g. to the “native” promoter). Alternatively, a gene of interest can be included in a recombinant nucleic acid molecule operably linked to a regulatory sequence that accompanies or is adjacent to another (e.g., a different) gene from the natural organism. Alternatively, a gene of interest can be included in a recombinant nucleic acid molecule operably linked to a regulatory sequence from a different, potentially only distantly related, organism. For example, regulatory sequences from other microbes (e.g., bacterial regulatory sequences from other species, bacteriophage regulatory sequences and the like) can be operably linked to a particular gene of interest.

In some embodiments, a regulatory sequence is a non-native or non-naturally-occurring sequence (e.g., a sequence which has been modified, mutated, substituted, derivatized, or deleted, including sequences which are chemically synthesized). Exemplary regulatory sequences include promoters, enhancers, termination signals, anti-termination signals and other expression control elements (e.g., sequences to which RNA polymerase, repressors or inducers bind and/or binding sites for transcriptional and/or translational regulatory proteins, including for example, sequences in the transcribed mRNA). Such regulatory sequences are well known in the art, and are described, for example, in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Regulatory sequences include those which direct constitutive expression of a nucleotide sequence in a microorganism (e.g., constitutive promoters and strong constitutive promoters), those which direct inducible expression of a nucleotide sequence in a microorganism (e.g., inducible promoters, for example, xylose inducible promoters) and those which attenuate or repress expression of a nucleotide sequence in a microorganism (e.g., attenuation signals or repressor sequences). It is also within the scope of the present invention to regulate expression of a gene of interest by removing or deleting regulatory sequences. For example, sequences involved in the negative regulation of transcription can be removed such that expression of a gene of interest is enhanced.

In some embodiments, a recombinant nucleic acid molecule of the present invention includes a nucleic acid sequence or gene that encodes at least one bacterial gene product (e.g., a methionine biosynthetic enzyme) operably linked to a promoter or promoter sequence. Exemplary promoters of the present invention include Corynebacterium promoters and/or bacteriophage promoters (e.g., bacteriophage which infect Corynebacterium). In one embodiment, a promoter is a Corynebacterium promoter, preferably a strong, Corynebacterium promoter (e.g., a promoter associated with a biochemical housekeeping gene, e.g., a relatively highly expressed housekeeping gene in Corynebacterium). In another embodiment, a promoter is a bacteriophage promoter. In some embodiments, the promoter is from the B. subtilis bacteriophage SP01 or the E. coli bacteriophage λ. In some embodiments, a promoter is selected from a P₁₅ or P₄₉₇ promoter having for example, the following respective sequences: (SEQ ID NO:3), and (SEQ ID NO:4). Additional promoters include tef (the translational elongation factor (TEF) promoter), the sod (superoxide dismutase) promoter, and pyc (the pyruvate carboxylase (PYC) promoter), which promote high level expression in Corynebacterium (e.g., Corynebacterium glutamicum). Additional examples of promoters, for example, for use in Gram positive microorganisms include, but are not limited to, amy and SP01 promoters. Additionally, for use in both Gram negative and Gram positive microorganisms, promoters including, but are not limited to, cos, tac, trp, tet, trp-tet, ipp, lac, lpp-lac, lacIQ, T7, T5, 13, gal, trc, ara, SP6, λ-PR or λ-PL, can be used.

In another embodiment, a recombinant nucleic acid molecule of the present invention includes a terminator sequence or terminator sequences (e.g., transcription terminator sequences). The term “terminator sequences” includes regulatory sequences that serve to terminate transcription of mRNA. Terminator sequences (or tandem transcription terminators) can further serve to stabilize mRNA (e.g., by adding structure to mRNA), for example, against nucleases.

In yet another embodiment, a recombinant nucleic acid molecule of the present invention includes sequences that allow for detection of the vector containing said sequences (i.e., detectable and/or selectable markers), for example, genes that encode antibiotic resistance sequences or that overcome auxotrophic mutations, for example, trpC, drug markers, fluorescent markers, and/or calorimetric markers (e.g., lacZ/β-galactosidase).

In yet another embodiment, a recombinant nucleic acid molecule of the present invention includes a native (found associated with the wild type gene) or an artificial or hybrid or composite ribosome binding site (RBS) or a sequence that is transcribed into an artificial RBS. The term “artificial ribosome binding site (RBS)” includes a site within an mRNA molecule (e.g., coded within DNA) to which a ribosome binds (e.g. to initiate translation) which differs from a native RBS (e.g., a RBS found in a naturally-occurring gene) by at least one nucleotide. In some embodiments, artificial RBSs include about 5-6, 7-8, 9-10, 11-12, 13-14, 15-16, 17-18, 19-20, 21-22, 23-24, 25-26, 27-28, 29-30 or more nucleotides of which about 1-2, 3-4, 5-6, 7-8, 9-10, 11-12, 13-15 or more differ from the native RBS. In some embodiments, RBS sequences include RBSI, (SEQ ID NO: 5 tctagaAGGAGGAGAAAACatg) and RBS 1284 (SEQ ID NO: 6: tctagaCCAGGAGGACATACAgtg) as described and used in the vectors of the present invention. (See Table III).

TABLE III
Plasmids designed to express B. subtilis
metI integrated at crtEb in
C. glutamicum.

| | | | |
|---|---|---|---|
| Plasmid name | Promoter | RBS | RBS sequence |

| | | | |
|---|---|---|---|
| pOM281 | P₄₉₇ | RBS1 | tctagaAGGAGGAGAAAACatg |
| (SEQ ID NO: 10) | | | (SEQ ID NO: 5) |
| pOM283 | ″ | eftU (1284) | tctagaCCAGGAGGACATACAgtg |
| (SEQ ID NO: 11) | | | (SEQ ID NO: 6) |
| pOM284 | P₁₅ | RBS1 | tctagaAGGAGGAGAAAACatg |
| (SEQ ID NO: 12) | | | (SEQ ID NO: 5) |
| pOM286 | ″ | eftU(1284) | tctagaCCAGGAGGACATACAgtg |
| (SEQ ID NO: 13) | | | (SEQ ID NO: 6) |

The present invention further features vectors (e.g., recombinant vectors) that include nucleic acid molecules (e.g., heterologous genes, heterologous nucleic acid sequences or recombinant nucleic acid molecules comprising said genes) as described herein. The term “recombinant vector” includes a vector (e.g., plasmid, phage, phagemid, virus, cosmid or other purified nucleic acid vector) that has been altered, modified or engineered such that it contains greater, fewer or different nucleic acid sequences than those included in the native or natural nucleic acid molecule from which the recombinant vector was derived. In some embodiments, the recombinant vector includes a biosynthetic enzyme-encoding gene or recombinant nucleic acid molecule including said gene, operably linked to regulatory sequences, for example, promoter sequences, terminator sequences and/or artificial ribosome binding sites (RBSs), as defined herein. In another embodiment, a recombinant vector of the present invention includes sequences that enhance replication in bacteria (e.g., origin of replication sequences). In one embodiment, replication-enhancing sequences function in E. coli. In another embodiment, replication-enhancing sequences are derived from pBR322.

In yet another embodiment, a recombinant vector of the present invention includes antibiotic resistance sequences. The term “antibiotic resistance sequences” includes sequences which promote or confer resistance to antibiotics on the host organism (e.g., Corynebacterium). In one embodiment, the antibiotic resistance sequences are selected from the group consisting of cat (chloramphenicol resistance) sequences, tet (tetracycline resistance) sequences, erm (erythromycin resistance) sequences, neo (neomycin resistance) sequences, kan (kanamycin resistance) sequences, amp (β-lactam antibiotic resistance sequences), and spec (spectinomycin resistance) sequences. Recombinant vectors of the present invention can further include homologous recombination sequences (e.g., sequences designed to allow recombination of the gene of interest into the chromosome of the host organism). For example, bioAD, bioB, or crtEb sequences can be used as homology targets for recombination into the host chromosome. It will further be appreciated by one of skill in the art that the design of a vector can be tailored depending on such factors as the choice of microorganism to be genetically engineered, the level of expression of gene product desired and the like.

### V. Carotenoid Biosynthesis and the Carotenoid Operon

Carotenoids are the general name for a group of fat-soluble, aliphatic hydrocarbons, that may also contain one or more oxygen atoms, consisting of a modified polyisoprene backbone that can act to cause pigmentation. They arise by way of the general isoprenoid biosynthetic pathways and are synthesized by plants, algae, some fungi and bacteria. Presently, more than 600 carotenoids are known to occur naturally. Carotenoids perform diverse functions besides providing characteristic coloration. Carotenoids can provide antioxidative protection, for example, protection against the effects of singlet oxygen and radicals. During photosynthesis, carotenoids can transfer absorbed radiant energy to chlorophyll molecules in a light harvesting function, dissipate excess energy via xanthophylls cycle in higher plants and certain algae, and quench excited-state-chlorophylls directly. Carotenoids might also provide protection against harmful radiation such as ultraviolet light. Recently, the structural role of carotenoids as the molecular glue of certain photosynthetic pigment-protein complexes has become evident. β-carotene and structurally related compounds serve as the precursor for Vitamin A, retina, and retinoic acid in mammals, thereby playing essential roles in nutrition, vision, and cellular differentiation, respectively. (Krubasik, P. et al, (2001) Eur. J. Biochem. 268:3702-3708; Armstrong G. A., (1994) J. Bacteriol. 176:4795-4802)

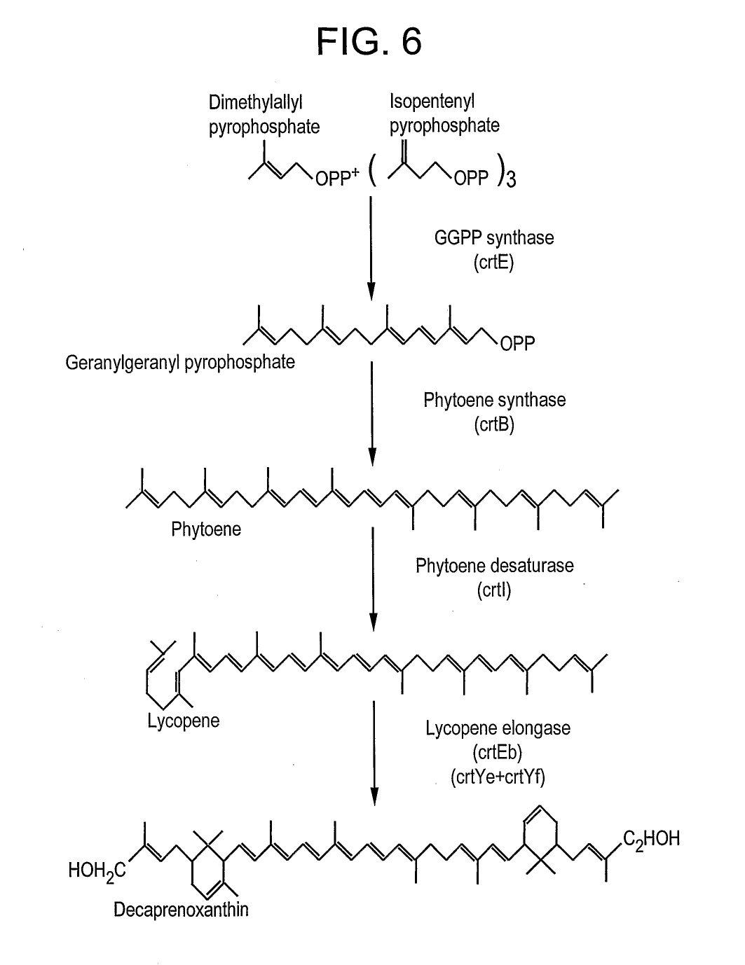

Many carotenoids contain a linear C40 hydrocarbon backbone that includes several, usually between 3-15, conjugated double bonds. In certain bacteria, however, C45 and C50 carotenoids are also produced. Decaprenoxanthin produced in C. glutamicum is one example of a C50 carotenoid (Krubasik, ibid). The number and arrangement of double bonds present largely determines the spectral properties of a given carotenoid, which typically absorbs light between 400 and 500 nm. The first step unique to the carotenoid branch of isoprenoid biosynthesis is the tail-to-tail condensation of two molecules of the C20 intermediate geranylgeranyl pyrophosphate (GGPP) to form phytoene (see FIG. 6). This acyclic hydrocarbon is the first C40 carotenoid produced and is common to all C40 carotegenic organisms. Depending upon the organism, phytoene is then converted to neurosporene or lycopene. Following this intermediate, biosynthetic pathways in carotegenic organisms diverge, yielding the variety of carotenoids present in nature. (Armstgrong, G. A. et al (1996) FASEB J. 10, 228-237)

Carotenoid synthesis is achieved through the progressive action of several enzymes functioning in a coordinated fashion to yield intermediate and final molecules. In e.g. C. glutamicum five enzymes function to produce the carotenoid decaprenoxanthin (see FIG. 6). The carotenoid operon is an attractive candidate for genetic engineering techniques for several reasons. The production of carotenoids is industrially significant because the utility of molecules such as lutein, astaxanthin, lycopene and beta carotene, etc. have long been known and there is increasing potential for the molecules as nutritional additives or supplements. For example, the use of lycopene as an antioxidant and anticancer agent has been the object of recent research. The operon may be easily manipulated to produce carotenoids of various structures based on providing and/or regulating the production of enzymes responsible for the steps in the carotenoid biosynthetic pathway of an organism. Further, the operon or organism may be manipulated to increase production of enzymes useful for the production of a desired carotenoid.

In addition, the operon may be used as a vehicle for the introduction of exogenous nucleic acid sequences through the use of integration cassettes. Such integration cassettes comprise nucleic acid sequences homologous to endogenous sequences of the operon. Through recombinative events the integration cassette inserts the exogenous sequence into the carotenoid operon of the target organism. The nucleic acid sequence may encode a protein of interest or it may contain non-coding sequence used to e.g. alter, disrupt or augment the functioning of the carotenoid operon.

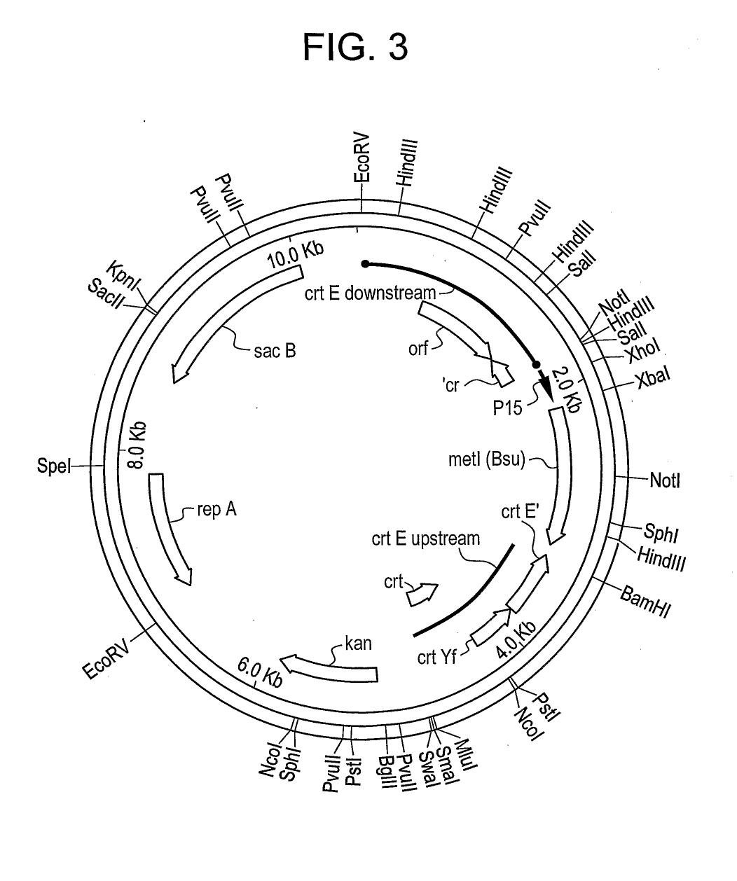

The present invention further relates to recombinant expression vectors that can integrate at the carotenoid operon (see FIG. 3) of Corynebacterium. The carotenoid operon is a genetic unit comprising several genes and gene regulatory elements responsible for the production of carotenoids. In particular, the inventors have developed expression vectors comprising integration cassettes that are useful for the introduction of heterologous nucleic acids or heterologous genes in the carotenoid operon. The inventors have designed the integration cassettes such that specific genes or regulatory sequences of the carotenoid operon may be targeted for disruption. Disruption of specific genes or regulatory sequences of the carotenoid operon yield different phenotypic results depending upon which step of the carotenoid pathway is disrupted or altered. C. glutamicum normally gives yellow colored colonies due to synthesis of decaprenoxanthin. For example, a block early in the pathway yields white colonies, and a block at lycopene elongase (encoded at the crtEb locus) yields pink colonies. Here the pink color is a result of the accumulation of lycopene instead of decaprenoxanthin. Finally, an insertion in marR, which encodes a putative negative regulator of the carotenoid operon, yields higher levels of total carotenoids, resulting in colonies darker or more intense in color. The inventors further demonstrate herein that the disruption of both the lycopene elongase (crtEb) locus and the marR locus yield significantly increased production of lycopene.

Taken together, the discoveries described herein provide for the generation of recombinant microorganisms that simultaneously produce increased levels of both methionine and lycopene or another carotenoid compound. This provides a distinct advantage due to the economy of using one organism for the increased production of two industrially significant compounds. The carotenoid may be obtained, without or with further purification from the cell mass left over from the fermentation.

Furthermore, vectors of the invention are useful in facilitating genetic engineering of microorganisms, because the color changes that accompany various engineering steps can help to identify the desired molecular events.

### IV. Culturing and Fermenting Recombinant Microorganisms

Microorganisms of the invention are particularly suitable for the production of fine chemicals, e.g., sulfur containing fine chemicals. Microorganisms as well as fermentation processes featuring such microorganisms, are preferably designed for the improved or enhanced production of fine chemicals, e.g., sulfur containing fine chemicals.

Process improvements can relate to methods regarding technical aspects of the fermentation, such as for example, stirring and oxygen supply, or due to the nutrient media composition, such as for example, sugar concentration during fermentation or to isolation techniques used in purifying the product, for example by ion exchange chromatography.

Means for improving the production of desired substances, e.g. sulfur-containing fine chemicals, include intrinsically improving the production titer or yield of a microorganism through, e.g., genetic engineering. Output of a desired substance (e.g. sulfur-containing fine chemicals) may be increased by modifying expression levels of an enzyme (or enzymes) involved in biosynthesis of the substance of interest. This may be achieved by, for example, modifying promoter or enhancer sequences responsible for driving expression of the biosynthetically important enzyme. Additionally, foreign promoter or enhancer sequences may be recombinantly introduced and confer preferred levels of expression of an endogenous enzyme or protein. In some instances the inserted regulatory sequences allow for constitutive or inducible expression of a target protein. Production of increased levels of a desired substance may also be achieved through the introduction of recombinantly modified genes that express proteins with improved characteristics. In certain instances, the genes coding native proteins are engineered such that the resultant proteins have desired characteristics, for example, higher affinity for substrate or faster reaction rate. Yet another way of achieving increased or improved production of a desired substance is through recombinantly introducing heterologous genes. Insertion of heterologous genes may have the benefit of supplementing or supplanting a native enzyme and thereby effecting the production of a particularly desired product of a biochemical pathway. In certain circumstances it may be advantageous to knock-out the expression of a native gene and introduce a heterologous gene, thus improving the production of a desired substance. Heterologous genes may also be introduced such that the production of a substance novel to the target microorganism is produced.

Of particular interest in improving the production of desired substances in microorganisms is the development of novel genetic engineering techniques for facilitating modification of a target organism. Generally, heterologous nucleic acid sequences are inserted into target organisms through the use of recombinant nucleic acid vectors. These vectors may be autonomously replicating and exist episomally or they may be designed such that the heterologous sequence is inserted into the host cells genome. Further, it is possible, and advantageous in certain circumstances, to design vectors that integrate site specifically. Integration vectors such as these may perform a two-fold function: They insert a desired heterologous gene and simultaneously ablate the function of a native, target gene sequence. The further development of vectors such as these provide means for facilitating the generation of recombinant microorganisms useful for the production of desired substances such as sulfur-containing fine chemicals.

The term “culturing” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured in solid media or semi-solid media. In some embodiments, a microorganism of the invention is cultured in a medium (e.g., a sterile, liquid medium) comprising nutrients essential or beneficial to the maintenance and/or growth of the microorganism (e.g., carbon sources or carbon substrate, for example carbohydrate, hydrocarbons, oils, fats, fatty acids, organic acids, and alcohols; nitrogen sources, for example, peptone, yeast extracts, meat extracts, malt extracts, urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, molybdenum, and/or cobalt salts; as well as growth factors such as amino acids, vitamins, growth promoters and the like).

Preferably, microorganisms of the present invention are cultured under controlled pH. The term “controlled pH” includes any pH that results in production of the desired product (e.g., methionine and/or lycopene). In one embodiment microorganisms are cultured at a pH of about 7. In another embodiment, microorganisms are cultured at a pH of between 6.0 and 8.5. The desired pH may be maintained by any number of methods known to those skilled in the art.

In some embodiments, microorganisms of the present invention are cultured under controlled aeration. The term “controlled aeration” includes sufficient aeration (e.g., supply of oxygen) to result in production of the desired product (e.g., methionine and/or lycopene). In one embodiment, aeration is controlled by regulating oxygen levels in the culture, for example, by regulating the amount of oxygen dissolved in culture media. For example, in some embodiments, aeration of the culture is controlled at least partially by agitating the culture. Agitation may be provided by a propeller or similar mechanical agitation equipment, by revolving or shaking the culture vessel (e.g. tube or flask) or by various pumping equipment. Aeration may be further controlled by the passage of sterile air or oxygen through the medium (e.g., through the fermentation mixture). Also microorganisms of the present invention are preferably cultured without excess foaming (e.g., via addition of antifoaming agents).

Moreover, microorganisms of the present invention can be cultured under controlled temperatures. The term “controlled temperature” includes any temperature which results in production of the desired product (e.g., methionine and/or carotenoid). In one embodiment, controlled temperatures include temperatures between 15° C. and 95° C. In another embodiment, controlled temperatures include temperatures between 15° C. and 70° C. In some embodiments, temperatures are between 20° C. and 55° C., more preferably between 28° C. and 44° C.

Microorganisms can be cultured (e.g., maintained and/or grown) in liquid media and preferably are cultured, either continuously or intermittently, by conventional culturing methods such as standing culture, test tube culture, shaking culture (e.g. rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g. a fermentation process). Fermentation processes of the present invention include, but are not limited to, batch, fed-batch and continuous processes or methods of fermentation. The phrase “batch process” or “batch fermentation” refers to a system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the additional provision that one or more substrates or supplements are added (e.g. added in increments or continuously) as the fermentation progresses. The phrase “continuous process” or “continuous fermentation” refers to a system in which a defined fermentation media is added continuously to a fermentor and an equal amount of used or “conditioned” media is simultaneously removed, preferably for recovery of the desired product (e.g., methionine and/or carotenoid). A variety of such processes has been developed and are well known in the art.

The phrase “culturing under conditions such that a desired compound is produced” includes maintaining and/or growing microorganisms under conditions (e.g., temperature, pressure, pH, duration, etc.) appropriate or sufficient to obtain production of the desired compound or to obtain desired yields of the particular compound being produced. For example, culturing is continued for a time sufficient to produce the desired amount of a compound (e.g., methionine and/or carotenoid). Preferably, culturing is continued for a time sufficient to substantially reach suitable production of the compound (e.g., a time sufficient to reach a suitable concentration of methionine and/or carotenoid). In one embodiment, culturing is continued for about 12 to 24 hours. In another embodiment, culturing is continued for about 24 to 36 hours, 36 to 48 hours, 48 to 72 hours, 72 to 96 hours, 96 to 120 hours, 120 to 144 hours, or greater than 144 hours. In yet other embodiments, microorganisms are cultured under conditions such that at least about 1 to 5 g/L or 5 to 10 g/L of compound are produced in about 48 hours, or at least about 10 to 20 g/L compound in about 72 hours. In yet another embodiment, microorganisms are cultured under conditions such that at least about 5 to 20 g/L of compound are produced in about 36 hours, at least about 20 to 30 μL compound are produced in about 48 hours, or at least about 30 to 50 or 60 g/L compound in about 72 hours.

The methodology of the present invention can further include a step of recovering a desired compound (e.g., methionine and/or carotenoid). The term “recovering” a desired compound includes extracting, harvesting, isolating or purifying the compound from culture media or cell mass. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), dialysis, filtration, concentration, crystallization, recrystallization, pH adjustment, lyophilization and the like.

In some cases, it is preferable that a desired compound of the present invention is “extracted”, “isolated” or “purified” such that the resulting preparation is substantially free of other media components (e.g., free of media components and/or fermentation byproducts). The language “substantially free of other media components” includes preparations of the desired compound in which the compound is separated from media components or fermentation byproducts of the culture from which it is produced. In one embodiment, the preparation has greater than about 80% (by dry weight) of the desired compound (e.g., less than about 20% of other media components or fermentation byproducts), more preferably greater than about 90% of the desired compound (e.g., less than about 10% of other media components or fermentation byproducts), still more preferably greater than about 95% of the desired compound (e.g., less than about 5% of other media components or fermentation byproducts), and most preferably greater than about 98-99% desired compound (e.g., less than about 1-2% other media components or fermentation byproducts).

In an alternative embodiment, the desired compound is not purified from the culture medium or microorganism, for example, when the microorganism is biologically non-hazardous (e.g., safe). For example, the entire culture (or culture supernatant) or cell mass can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized. In yet another embodiment the cell mass (after separation from the culture supernatant) is dried, lyophilized, or used directly, for example as a feed additive. The product obtained by the present invention can include in addition to sulfur-containing fine chemical, e.g., methionine, other components of the fermentation broth and cell mass, e.g. phosphates, carbonates, remaining carbohydrates, biomass, complex media components, carotenoids, etc.

In some embodiments, a production method of the present invention results in production of the desired compound at a significantly high yield. The phrase “significantly high yield” includes a level of production or yield which is sufficiently elevated or above what is usual for comparable production methods, for example, which is elevated to a level sufficient for commercial production of the desired product (e.g., production of the product at a commercially feasible cost). In one embodiment, the invention features a production method that includes culturing a recombinant microorganism under conditions such that the desired product (e.g. methionine and/or carotenoid ) is produced at a level greater than 2 g/L for a soluble product such as methionine, or greater than 0.1 mg/L for a poorly soluble product (e.g. a carotenoid). In another embodiment, the invention features a production method that includes culturing a recombinant microorganism under conditions such that the desired product (e.g., methionine) is produced at a level greater than 10 g/L, and when present, the carotenoid compound at a level of 1 mg/L or greater. In another embodiment, the invention features a production method that includes culturing a recombinant microorganism under conditions such that the desired product (methionine) is produced at a level greater than 20 g/L. In yet another embodiment, the invention features a production method that includes culturing a recombinant microorganism under conditions such that the desired product (methionine) is produced at a level greater than 30 g/L. In yet another embodiment, the invention features a production method that includes culturing a recombinant microorganism under conditions such that the desired product (e.g., methionine) is produced at a level greater than 40 g/L. In yet another embodiment, the invention features a production method that includes culturing a recombinant microorganism under conditions such that the desired product (e.g., methionine) is produced at a level greater than 50 g/L. In yet another embodiment, the invention features a production method that includes culturing a recombinant microorganism under conditions such that the desired product (e.g., methionine) is produced at a level greater than 60 g/L. The invention further features a production method for producing the desired compound that involves culturing a recombinant microorganism under conditions such that a sufficiently elevated level of compound is produced within a commercially desirable period of time.

Depending on the biosynthetic enzyme or combination of biosynthetic enzymes manipulated, it may be desirable or necessary to provide (e.g., feed) microorganisms of the present invention at least one biosynthetic precursor such that the desired compound or compounds are produced. The terms “biosynthetic precursor” and “precursor” include an agent or compound which, when provided to, brought into contact with, or included in the culture medium of a microorganism, serves to enhance or increase biosynthesis of the desired product.

Another aspect of the present invention includes biotransformation processes which feature the recombinant microorganisms described herein. The term “biotransformation process”, also referred to herein as “bioconversion processes”, includes biological processes which results in the production (e.g., transformation or conversion) of appropriate substrates and/or intermediate compounds into a desired product (e.g., methionine and/or carotenoid).

The microorganism(s) and/or enzymes used in the biotransformation reactions are in a form allowing them to perform their intended function (e.g., producing a desired compound). The microorganisms can be whole cells, or can be only those portions of the cells necessary to obtain the desired end result. The microorganisms can be suspended (e.g., in an appropriate solution such as buffered solutions or media), rinsed (e.g., rinsed free of media from culturing the microorganism), acetone-dried, immobilized (e.g., with polyacrylamide gel or k-carrageenan or on synthetic supports, for example, beads, matrices and the like), fixed, cross-linked or permeablized (e.g., have permeablized membranes and/or walls such that compounds, for example, substrates, intermediates or products can more easily pass through said membrane or wall).

This invention is further illustrated by the following examples which should not be construed as limiting. The contents of all references, patents and published patent applications cited throughout this application are incorporated herein by reference.

### Example 1

### Installation of the Bacillus subtilis metI Gene into C. glutamicum Strains

A clone of the B. subtilis metI gene was obtained by polymerase chain reaction and expressed in various C. glutamicum methionine producing strains. After amplifying metI by PCR, four different plasmids were constructed to constitutively express metI following integration at the crtEb locus (see Example 3). Two promoters, P₄₉₇ and P₁₅, were combined with two ribosome binding sites, RBS1, and RBS 1284, to give four combinations, which are listed in Table 2. One representative plasmid from this set, pOM284, is illustrated in FIG. 3. All of the plasmids complemented an E. coli metB mutant.

All four plasmids were transformed into OM99 (described in co-pending patent application, 60/700,699 “Methionine Producing Recombinant Microorganisms,” filed on Jul. 18, 2005). Four isolates of each of the Campbell-in strains were assayed for methionine production in shake flasks using a molasses based medium (Table IV). All four plasmids lead to an increase in methionine production. The largest improvement came from pOM284, which contains metI expressed from P₁₅ and RBS1. In this case, methionine production increased from about 1.6 g/l to about 2.2 g/l, or about 37%. This increase was interpreted to be due to either an increase in specific activity of the MetY-like activity, the MetB-like activity, or to feedback resistance, or to some combination of these. O-acetyl-homoserine sulfhydrylase enzyme assays in crude extracts of E. coli metB⁻ containing pOM284 showed that metI was, in fact, resistant to inhibition by methionine at concentrations up to 10 mM (see Example 2).

TABLE IV
Methionine production by derivatives of OM99 containing Campbelled-in
metI plasmids-grown for 48 hours in shake flasks in molasses medium.
All titers are given in grams per liter.

| | | | | O-Ac- | | |
|---|---|---|---|---|---|---|
| Strain | | Promoter | RBS | Hse | Met | Lys |
| OM99/pCLIK | -1 | — | — | 2.0 | 1.6 | 2.4 |
| (empty vector) | -2 | — | — | 1.7 | 1.5 | 2.3 |
| OM99/pOM281 | -1 | P₄₉₇ | RBS1 | 1.0 | 1.9 | 3.0 |
| | -2 | ″ | ″ | 0.9 | 2.0 | 3.2 |
| | -3 | ″ | ″ | 0.8 | 1.9 | 2.9 |
| | -4 | ″ | ″ | 0.8 | 1.9 | 2.8 |
| OM99/pOM283 | -1 | P₄₉₇ | 1284 | 0.8 | 2.0 | 2.6 |
| | -2 | ″ | ″ | 0.9 | 2.0 | 3.1 |
| | -3 | ″ | ″ | 1.1 | 1.9 | 2.7 |
| | -4 | ″ | ″ | 0.9 | 1.9 | 2.7 |
| OM99/pOM284 | -1 | P₁₅ | RBS1 | 0.5 | 2.3 | 3.0 |
| | -2 | ″ | ″ | 0.6 | 2.3 | 2.9 |
| | -3 | ″ | ″ | 0.5 | 2.2 | 2.7 |
| | -4 | ″ | ″ | 0.5 | 2.2 | 2.8 |
| OM99/pOM286 | -1 | P₁₅ | 1284 | 0.7 | 2.2 | 2.7 |
| | -2 | ″ | ″ | 0.5 | 2.1 | 2.7 |
| | -3 | ″ | ″ | 0.5 | 2.1 | 2.8 |
| | -4 | ″ | ″ | 0.5 | 2.3 | 3.1 |

The derivative of OM99 transformed with pOM284 was Campbelled-out to give a new strain named OM134C. In shake flasks, OM134C gave a 40% increase in methionine production relative to OM99, which was similar to that of the Campbelled-in intermediate, OM99/pOM284 (Table V). The O-acetyl-homoserine titer of OM134C was down from about 1.2 g/l to about 0.3 g/l, which is consistent with the presence of a more active O-acetyl-homoserine sulfhydrylase and/or a more active cystathionine synthase.

TABLE V
Methionine production by OM134C, a Campbelled-out derivative
of OM99 containing P₁₅ RBS1 metI integrated at crtEb,
grown for 48 hours in shake flasks in molasses medium.

| | | | O-Ac- | | |
|---|---|---|---|---|---|
| Strain | Promoter | RBS | Hse | Met | Lys |
| OM99 | — | — | 1.1 | 1.7 | 3.3 |
| ″ | — | — | 1.2 | 1.8 | 3.3 |
| OM134C-7 | P₁₅ | RBS1 | 0.3 | 2.5 | 3.3 |
| ″ | ″ | ″ | 0.3 | 2.4 | 3.2 |

All titers are given in grams per liter.

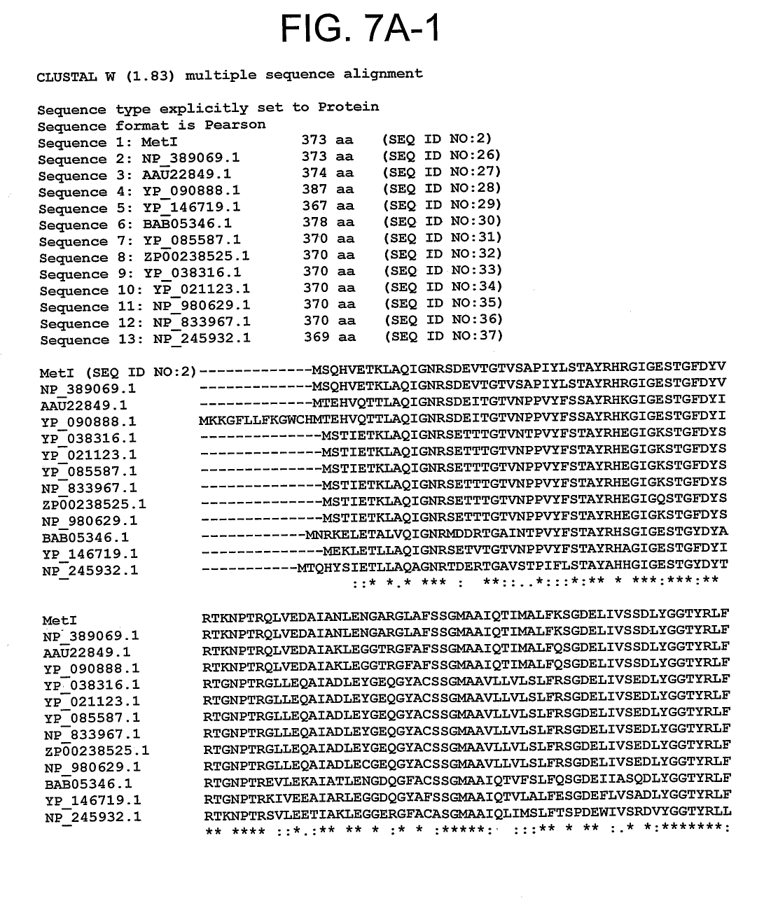

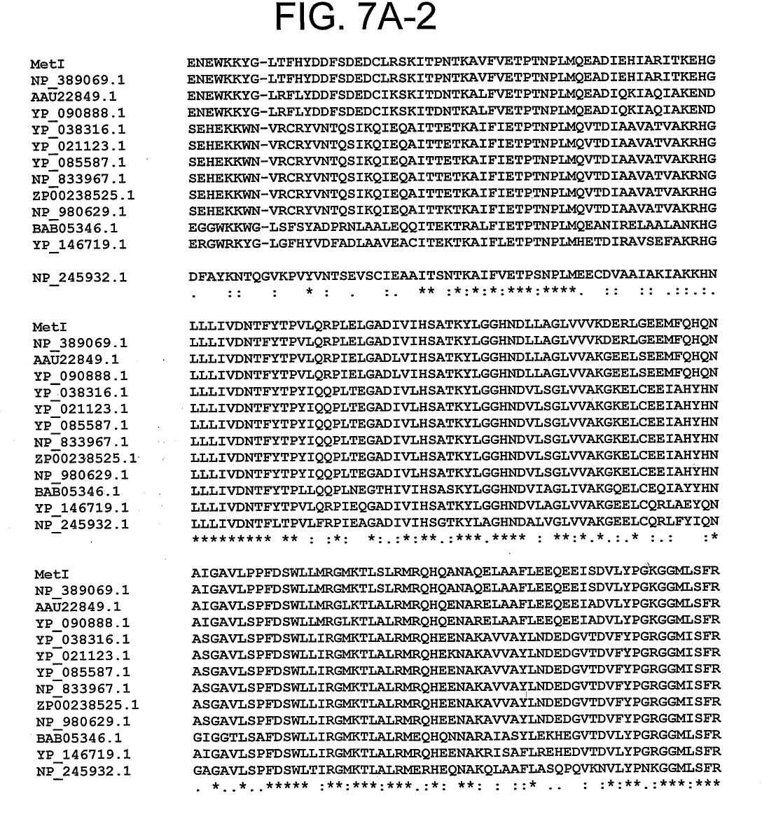

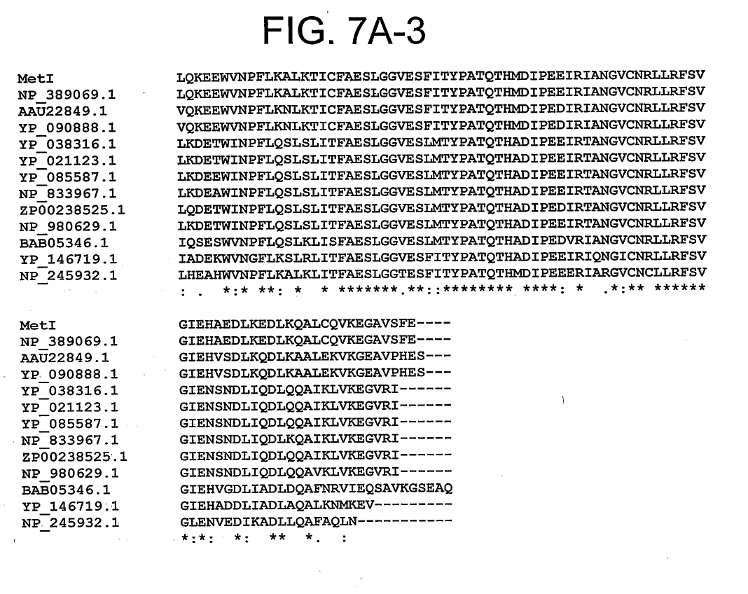

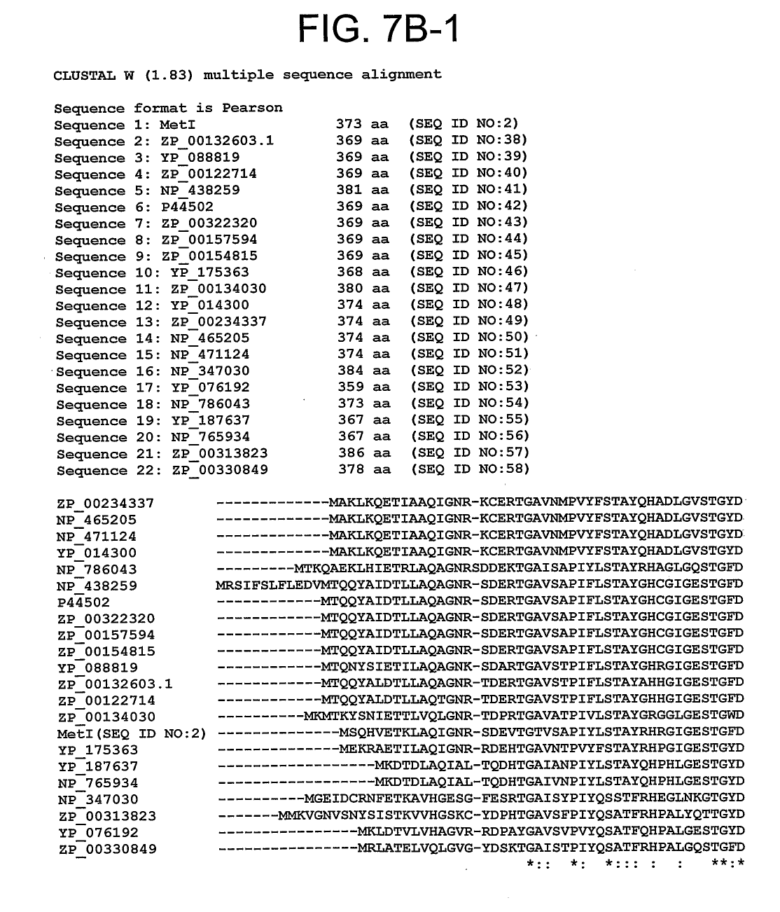

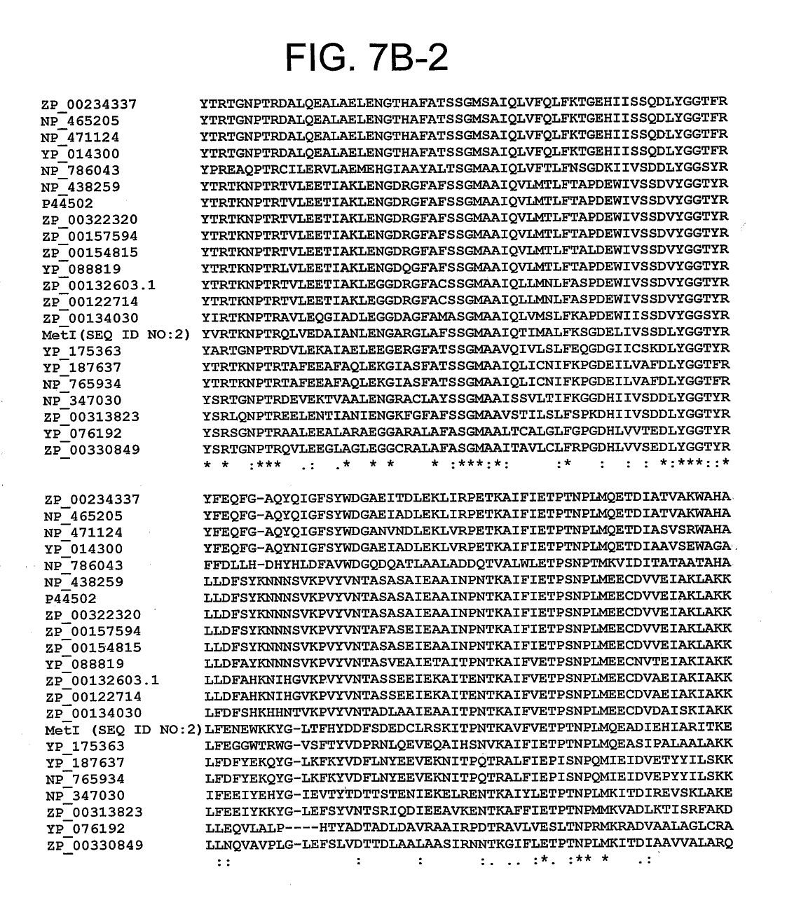

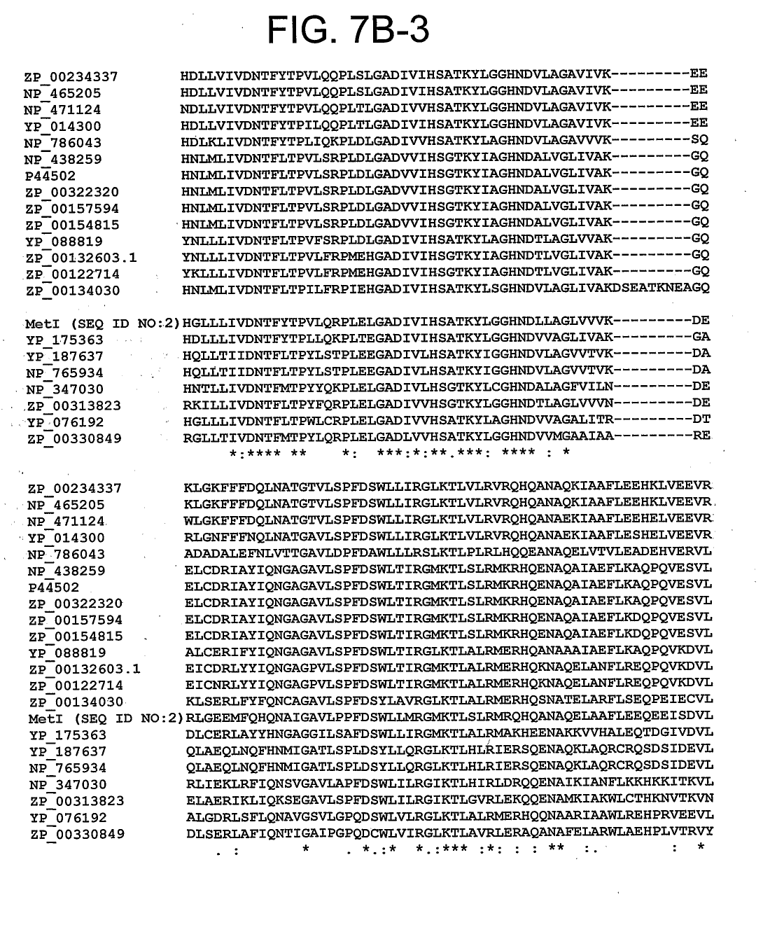

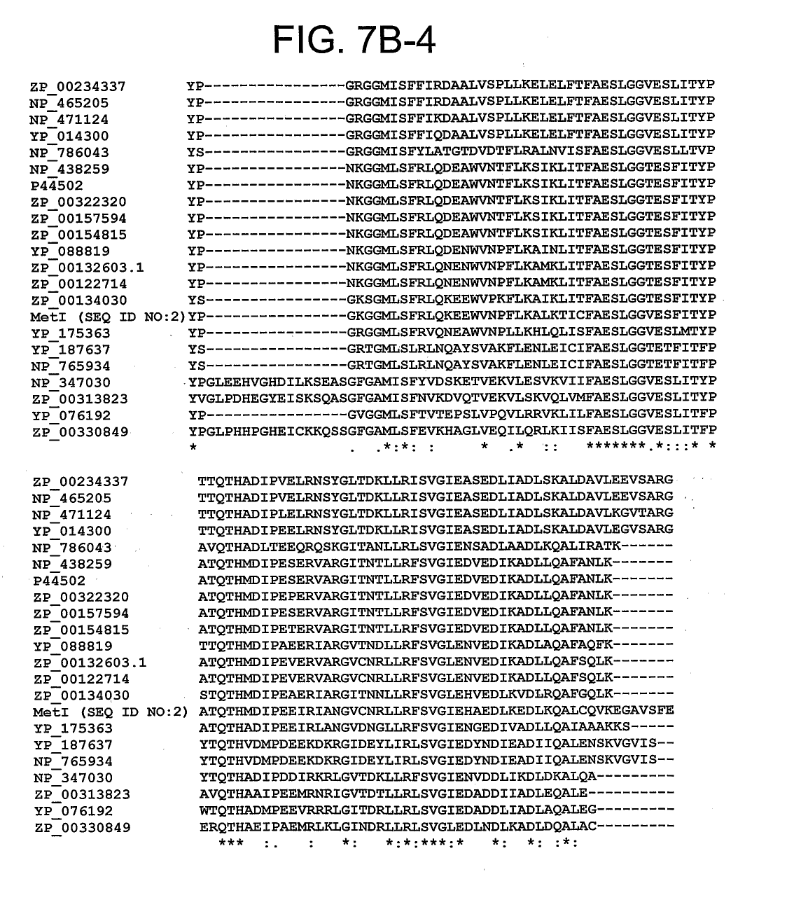

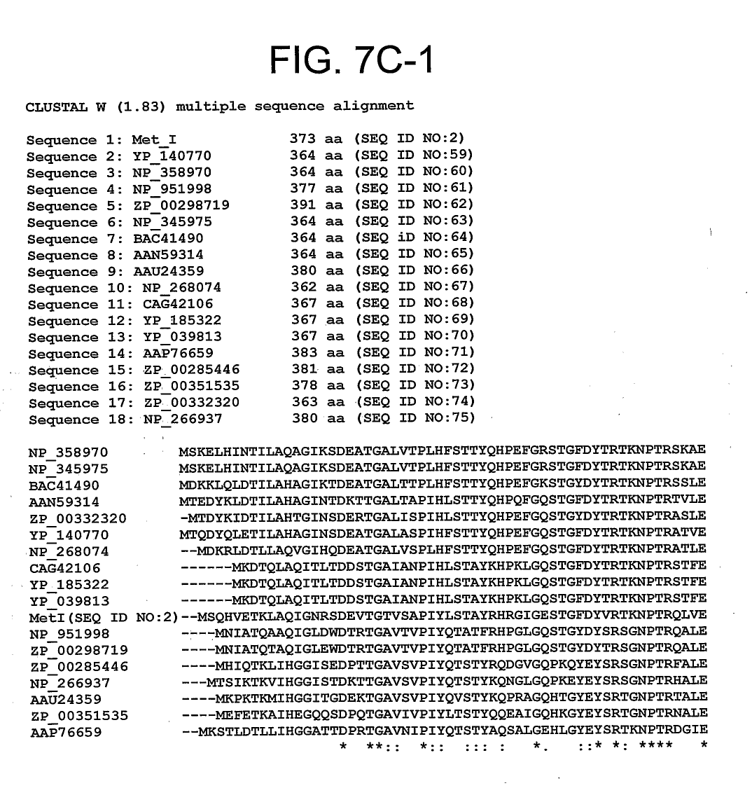

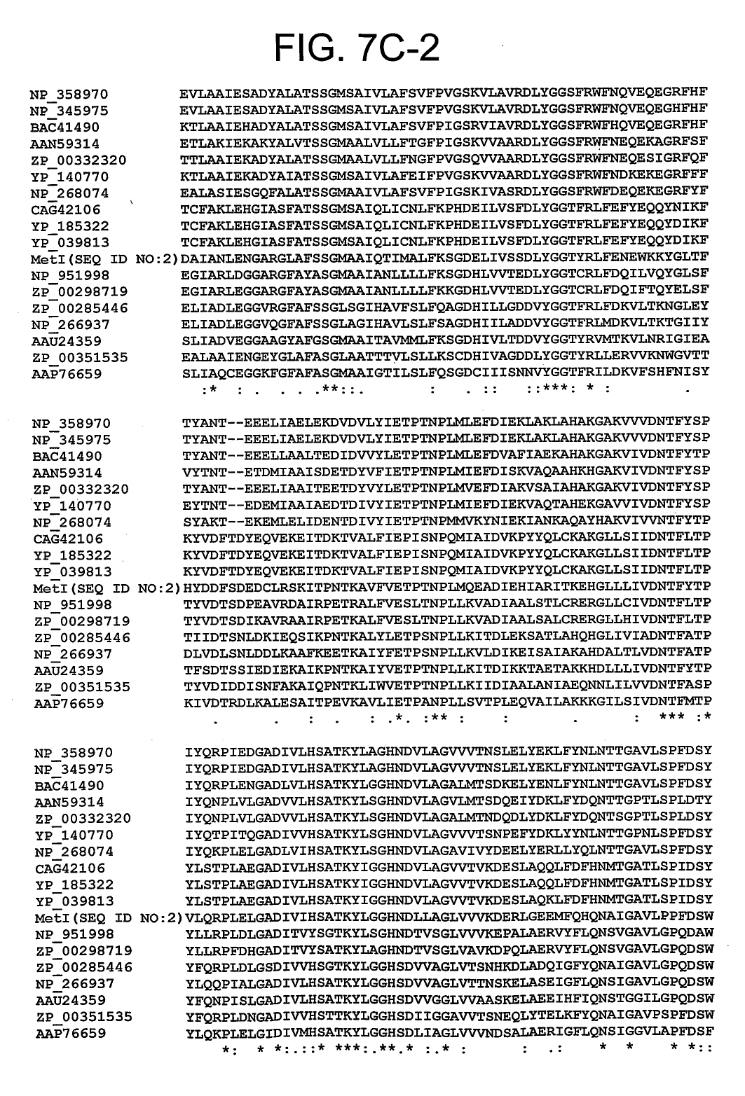

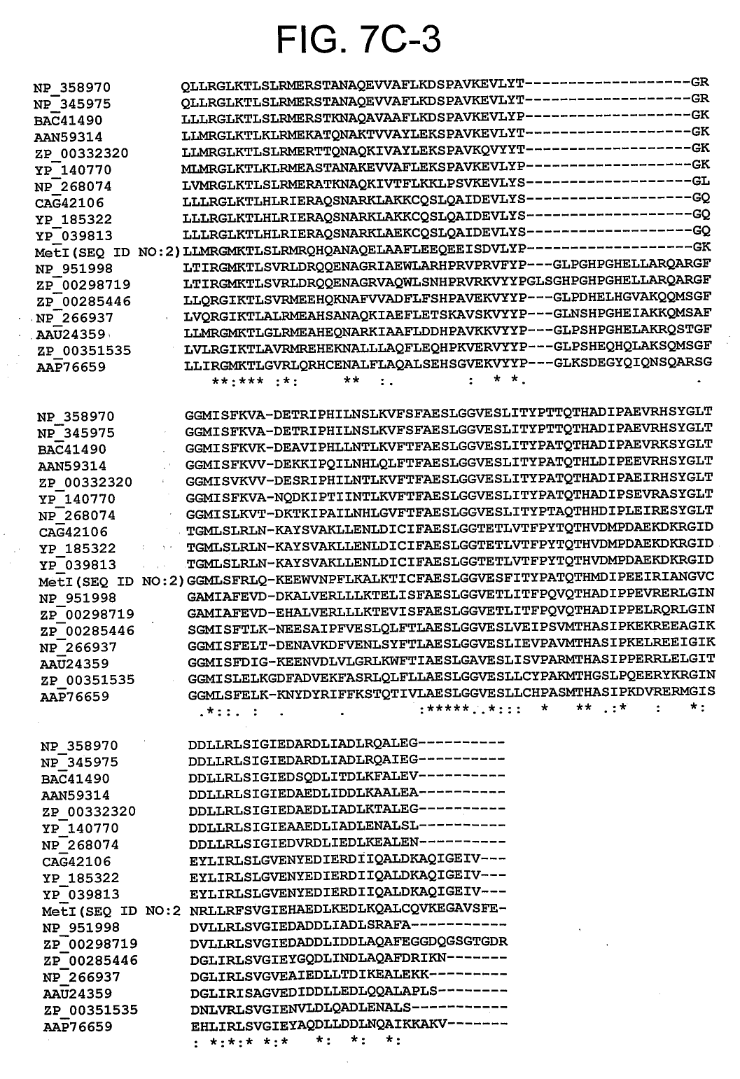

Sequences of various promoters useful in the construction of strains of the present invention are set forth in SEQ ID NO:16 (promoter P1284); SEQ ID NO: 17 (promoter P3119); SEQ ID NO:18 (promoterphage lambda P_{R}); and SEQ ID NO:19 (promoter phage lamdda P_{L}). Additionally, the amino acid sequence of the Bacillus subtilis metI protein was used to search for the closest known sequences. FIG. 7A-C depicts multiple sequence alignments between the B. subtilis metI protein (SEQ ID NO:2) and fifty closest sequences (SEQ ID NOs:26-75), by way of sequence identity, found in NCBI's GENBANK® database.

### Example 2

### Determination of O-Acetyl-Homoserine Sulfhydrylase Enzyme Activity of MetY from Corynebacterium glutamicum and metI from Bacillus subtilis as a Function of Methionine Concentration

The metI gene coded on the E. coli-C. glutamicum plasmid shuttle vector pOM284 (SEQ ID:12), and the metY gene coded on the E. coli-C. glutamicum plasmid shuttle vector pH357 (SEQ ID:15), were transformed by standard transformation technology into the metB deficient E. coli strain CGSC4896 from the Coli Genetic Stock Center (Yale University, USA) and were selected by growth on LB plus 25 mg/l kanamycin. The transformed E. coli strain containing pOM284 grew on minimal glucose medium lacking methionine, demonstrating that metI can utilize O-succinylhomoserine as a substrate.

E. coli strains carrying the metI or metY gene were grown in liquid LB medium with 25 mg/l kanamycin. Cells were harvested and cell lysates from pellets were obtained using the Ribolyzer protocol and machine (Hybaid, UK). Cell extracts were centrifuged to obtain a soluble supernatant fraction of cytosolic protein. The method to determine the O-acetyl-homoserine sulfhydrylase activity in cell extracts was performed essentially as described in Yamagata, Methods in Enzymology, 1987, Vol. 143 pp 479-480. Cell extracts were added to a buffer of 100 mM KH₂PO₄ (pH 7.2) containing 5 mM O-acetyl-homoserine and 200 μM pyridoxal phosphate. For the analysis of the effect of methionine on the enzymatic activity, L-methionine was added to the indicated final mM concentrations. The reaction was initiated by addition of Na-sulphide solution to a final concentration of 4 mM. After a 15 minute incubation at 30° C., the reaction was terminated and acidified by addition of 1/10 volume of 30% trichloroacetic acid. After centrifugation (5 minutes at 13,000 rpm) to remove precipitated protein, incubation at reduced atmospheric pressure in a Speed-Vac evaporator was performed to deplete residual H₂S. The sulphide depleted solution was reacted with cyanide and nitroprusside as described in Yamagata supra. Absorption at 520 nm was determined and background corrected.

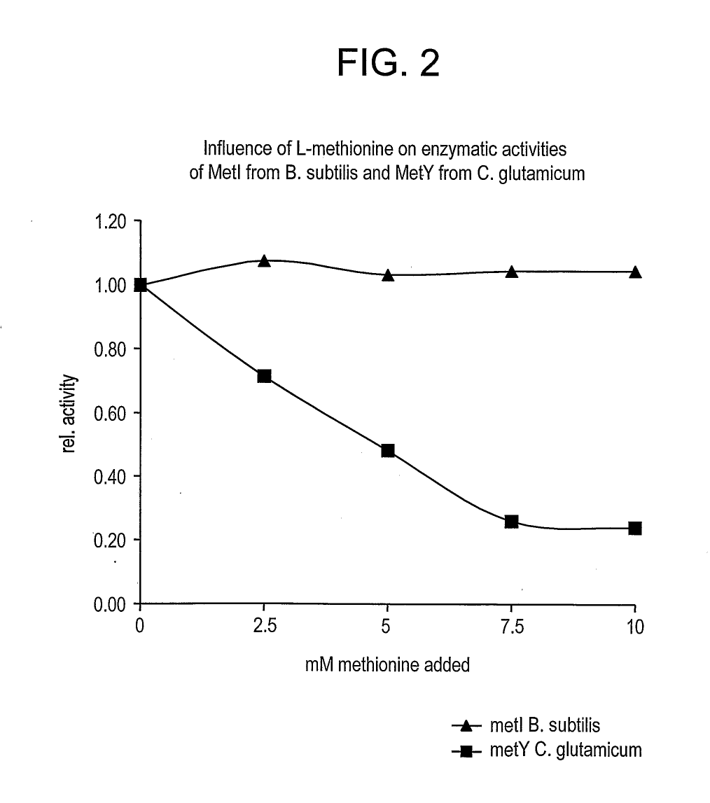

Enzymatic activities in the presence of methionine are expressed as relative values compared to the activities in the absence of added methionine, which is set at 1 (see FIG. 2). The E. coli strain CGSC4896 without addition of plasmid DNA showed no measurable enzymatic O-acetyl-homoserine sulfhydrylase activity.

It is clear from the results depicted in FIG. 2 that the O-acetyl-homoserine sulfhydrylase activity of the Bacillus subtilis metI enzyme is resistant to inhibition by methionine up to at least 10 mM methionine, while the O-acetyl-homoserine sulfhydrylase activity of the C. glutamicum MetY enzyme is inhibited by methionine in the range of 2.5 to 10 mM, with a 50% inhibition at about 5 mM. 5 mM is a methionine concentration that is likely to exist in the cytoplasm of cells that are engineered to overproduce methionine.

### Example 3

### Improvement of the In Vivo O-Acetylhomoserine Sulfhydrylase and O-Succinylhomoserine Sulfhydrylase Activity of metI Enzyme

Although metI from B. subtilis has O-acetylhomoserine sulfhydrylase activity in an in vitro enzyme assay, as depicted in examples 1 and 2 above, the in vivo activity of MetI was not sufficient to support growth of an E. coli or a C. glutamicum strain that lacked the transsulfuration pathway.

Plasmid pOM150 (SEQ ID NO:20) was constructed by substituting the P₁₅metI cassette from pOM284 (SEQ ID NO:12) for the P₄₉₇metY cassette of pH357 (SEQ ID NO:15).

E. coli strain MW001 (metB, metC162::Tn10) was constructed by P1vir transduction of the metC162::Tn10 allele from E. coli strain CGSC 7435 into CGSC 4896 (metB) and selecting for tetracycline resistance. MW001 lacks both the transsulfuration pathway and the direct sulfhydrylation pathway for methionine synthesis.

C. glutamicum strain OM175 was constructed by deleting portions of metB, metC, and metY from OM99, using serial Campbelling in and Campbelling out of plasmids pH216 (SEQ ID NO: 21), pOM115 (SEQ ID NO: 22), and pH215 (SEQ ID NO: 23), respectively. OM175 lacks both the transsulfuration pathway and the direct sulfhydrylation pathway for methionine synthesis.

MW001 and OM175 were each transformed with pOM150, selecting for kanamycin resistance at 25 mg/l. The transformants were streaked on Petri plates containing methionine free medium, as (described in U.S. Provisional Patent Application 60/700,557, filed Jul. 18, 2005, incorporated by reference herein. Neither transformant grew on methionine free medium, even though the in vitro sulfhydrylation activity of metI suggested that the transformants should have been endowed with the direct sulfhydrylation pathway by MetI.

In order to increase the in vivo direct sulfhydrylation activity of Met, MW001/pOM150 strain was subjected to ultraviolet mutagenesis and selection for growth on methionine free plates. Mutant strains that grew well were isolated. Plasmid DNA was isolated from several independent mutants and the purified plasmid DNAs were retransformed into naïve MW001 and OM175. Plasmids isolated from several different mutants gave transformants in both species (MW001 and OM175) that grew on methionine free medium, and the MW001 transformants of those plasmids grew at the same rate as the original mutant isolates, showing that the mutation that conferred growth was plasmid borne.

Two of the new mutant plasmids were named pOM150*-2 and pOM150*-14, respectively. The DNA sequence of the metI region of both plasmids was determined, and both contained the same single base mutation that changed the serine codon (AGC) at amino acid position 308 of metI (counting the ATG start codon as amino acid number one) to an asparagine codon (AAC). It is worth noting that the MetY, which has direct sulfhydrylation activity, contains asparagine at the homologous amino acid position, as a result, the mutation identified in the pOM150* plasmids rendered the MetI sequence more MetY-like.

A plasmid named pOM148*-1 (SEQ ID NO: 24) is a relative of pOM150*-14 that contains the same P₁₅metI (S308N) cassette as pOM150*-14, but no metX gene. Unlike pOM150*-2, which was isolated in MW001, pOM148*-1 was originally isolated in OM175 after ultraviolet mutagenesis, selection on methionine free plates, isolation of the plasmid, and transformation into naïve OM175 and MW001. E. coli strain MW001/pOM148*-1, which presumably produces O-succinylhomoserine, but no O-acetylhomoserine, still grows well on methionine free medium.

Taken all together, these results led to the conclusion that the novel mutant version of metI (S308N) has increased O-acetylhomoserine sulfhydrylase and O-succinylhomoserine sulfhydrylase activity in vivo in both C. glutamicum and E. coli, which is useful for enhancing methionine biosynthesis.

### Example 4

### Development of Vectors for Integrating Gene Expression Cassettes at the Carotenoid Biosynthetic Operon of C. glutamicum

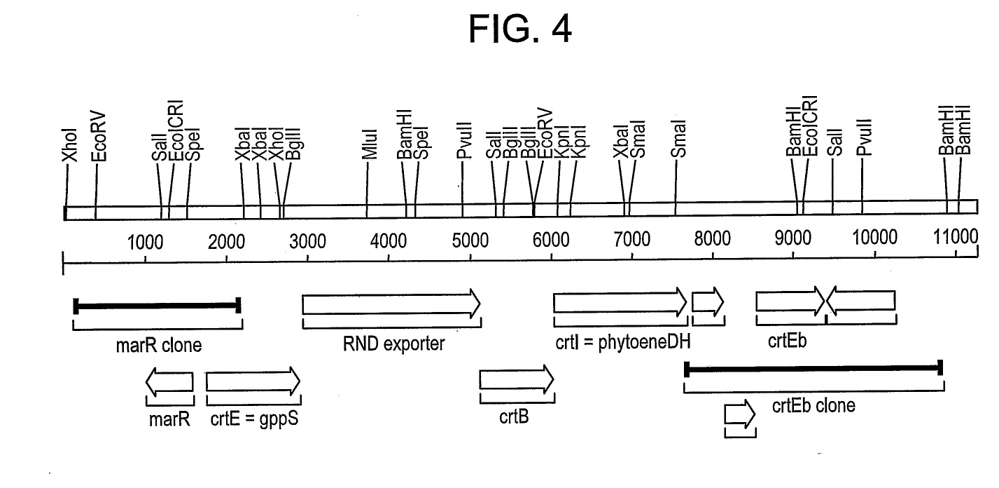

C. glutamicum colonies typically become yellow in color after 48 hours on minimal or rich plates. This yellow color is reported to be due to accumulation of the C50 carotenoid, decaprenoxanthin (Krubasik et al., 2001, Eur. J. Biochem. 268:3702-8). The enzymes which catalyze the biosynthesis of decaprenoxanthin from the isoprenoid precursors are encoded by a single operon that was characterized by transposon mutagenesis, cloning, and sequencing (Krubasik et al., ibid). We predicted that this operon (FIG. 4) was not essential for C. glutamicum, so it would be a convenient, and potentially useful, locus for insertion of gene expression cassettes. In particular, insertions at specific places in the operon would alter the carotenoid pathway, which in turn would lead to color changes in the colonies. For example, a block early in the pathway would lead to white colonies, and a block at lycopene elongase would lead to accumulation of lycopene instead of decaprenoxanthin, which would make the colonies pink instead of yellow. Finally, an insertion in marR, which encodes a putative negative regulator of the carotenoid operon, would lead to higher levels of carotenoids, which would make the colonies darker or more intense in color.

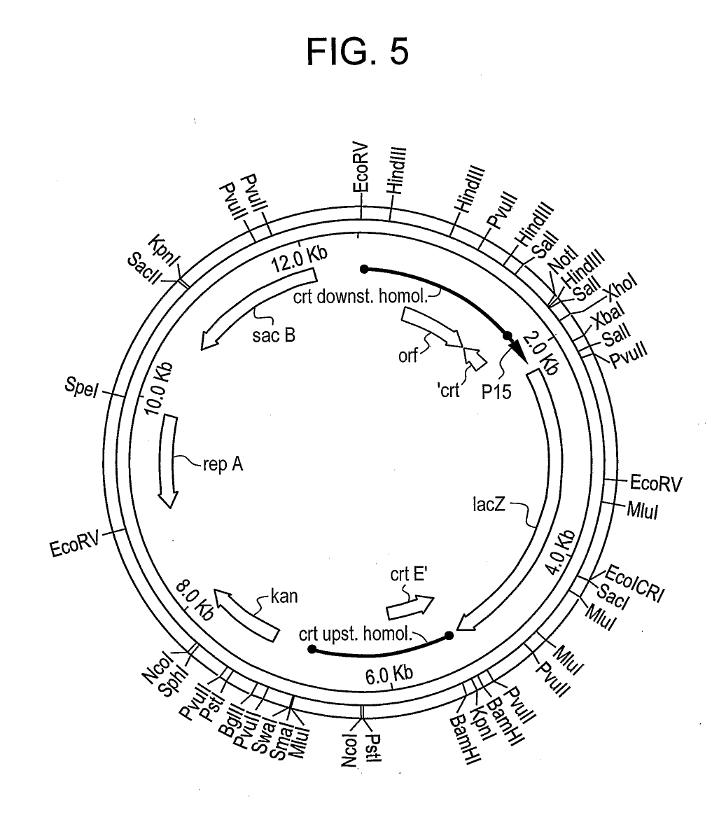

Two sets of integration vectors were designed to integrate cassettes at either crtEb (lycopene elongase) or marR (negative regulator). One member of each set contained a P₄₉₇lacZ expression module, and the other contained a P₁₅-lacZ expression module. One representative of these vectors, pOM246 (P₁₅-lacZ at crtEb) is shown in FIG. 5 (SEQ ID NO:14). The set of four vectors is summarized in Table VL Integration of the cassettes at crtEb produced pink colonies, which made it more efficient to pick “Campbell outs” that retained the desired insert.

Inserts at marR produced colonies that had a deeper yellow color than the parent. A combination of insertion at marR and insertion at crtEb leads to an increase in lycopene production.

### Example 5

### Co-Production of a Non-Carotenoid Compound and a Carotenoid Compound

As discussed herein, the plasmids and strains described herein, in addition to being useful in strain construction, can be used in methods for increasing the commercial value of a fermentation process by co-producing an amino acid, or other non-carotenoid compound of commercial interest, together with a carotenoid compound. Thus, for example, strain OM134C (see Example 1) produces both methionine and lycopene. The methionine is secreted into the medium of a liquid culture, while the lycopene remains bound to the cell mass. Upon centrifugation, the cells form a pink pellet, and the lycopene contained therein can be extracted, for example by suspending the cells in a mixture of methanol:chloroform (1:1 by volume). For some applications, for example, astaxanthin for salmon feed, the cell mass can be simply dried into a solid or powder and mixed with the feed to provide a source of carotenoid, protein, and vitamins.

Carotenoids (for example, but not limited to, lycopene, astaxanthin, β-carotene, lutein, zeaxanthin, canthaxanthin, decaprenoxanthin, and bixin, etc.) can accordingly be obtained from the spent cell mass from C. glutamicum or other fermentations where the first product is an amino acid or other non-carotenoid compound, thus saving the cost of a fermentation dedicated only to carotenoid production. Insertions described here lead to an increase in carotenoid levels, which make the carotenoid economically attractive to harvest as a byproduct. Carotenoids other than lycopene and decaprenoxanthin can also be produced by introduction of the appropriate biosynthetic genes, from sources well known in the art, using techniques well known in the art, for example, genes for astaxanthin and beta-carotene biosynthesis can be obtained by PCR from Phaffia rhodozyma or Xanthophyllomyces dendrorhous (Verdoes et al. (2003) Appl. Env. Microbiol. 69:3728-3738, or from Erwinia uredovora and Agrobacterium aurantiacum (Miura et al. (1998) Appl. Env. Microbiol. 64:1226-1229). The necessary genes to convert lycopene to beta-carotene, astaxanthin, etc. can be obtained from the above mentioned sources, or other appropriate sources, and expressed singly in C. glutamicum as described herein for metI or as an operon, or as part of an operon.

Without wishing to be bound by theory, it is contemplated that methods described herein can be extended to the production of amino acids other than methionine, or compounds other than amino acids, or non-carotenoid compounds and carotenoids other than decaprenoxanthin and lycopene, and using other organisms in addition to C. glutamicum. Additionally, methods encompassed by this invention can be used for the co-production of an amino acid or other non-carotenoid compound and a carotenoid compound in a single fermentation reaction. Examples of other amino acids include, but are not limited to, lysine, glutamic acid, threonine, isoleucine, leucine, alanine, phenylalanine, tyrosine, tryptophan, cysteine, homoserine, homocysteine, and salts thereof. Examples of other carotenoids include, but are not limited to, β-carotene, astaxanthin, lutein, zeaxanthin, canthaxanthin, and bixin. Any organism that can be engineered to overproduce an amino acid can be also engineered to co-produce a carotenoid. In general, the titer of the amino acid will be higher than that of the carotenoid, and the amount of carbon flux into the carotenoid will be small enough so that a major impact on the amino acid titer will not be obtained. Also, in some cases the production or overproduction of a carotenoid will actually enhance the titer of the amino acid being produced, since the carotenoid will give some protection to the producing organism against oxidative damage. Examples of organisms other than C. glutamicum that can be engineered to co-produce a non-carotenoid compound together with a carotenoid compound include other genera and species of bacteria, yeasts, filamentous fungi, archaea, and plants. The only requirement is that the organism is able to be engineered to produce the two compounds at commercially attractive levels.

In addition, increasing the value of a fermentation by co-producing a carotenoid (a second compound) can be extended to organisms and fermentations where the first compound of interest is a compound other than an amino acid. Such compounds include, for example, but are not limited to, methane, hydrogen, lactic acid, 1,2-propane diol, 1,3-propane diol, ethanol, methanol, propanol, acetone, butanol, acetic acid, propionic acid, citric acid, itaconic acid, glucosamine, glycerol, sugars, vitamins, therapeutic enzymes, research and industrial enzymes, therapeutic proteins, research and industrial proteins, and various salts of any of the above listed compounds. It is well known in the art that such compounds can be produced by fermentation, and that organisms can be engineered, selected, or screened to overproduce such compounds at commercially attractive levels. Further V value can be added to the fermentation process by co-producing a carotenoid that binds to cell mass or to a material that can be separated from soluble material after cell disruption. In many, but not all, cases, the first compound of interest will be water soluble to at least 0.5 g/l and secreted into the culture supernant, and the second compound of interest, for example a carotenoid, will be poorly soluble in water and will remain bound to the cell mass or to material concentratable from the culture or from disrupted cells by centrifugation or other means (for example evaporation, filtration, ultrafiltration, etc.). In some cases, the first compound will be a gas such as methane or hydrogen that can be easily separated from the carotenoid.

### Example 6

### Further Increasing the Production of Carotenoids

As discussed above in Example 4, carotenoid production can be increased by creating a non-functional allele (for example an insertion, deletion, or point mutation) in a gene that encodes a negative regulator of carotenoid biosynthesis, such as the marR gene in C. glutamicum. This approach leads to constitutive transcription of a carotenoid biosynthetic gene or operon. However, an even further increase in the level of carotenoid synthesis can be obtained by installing a promoter that is stronger than the native promoter (even in its derepressed state) upstream of the carotenoid gene or operon. Plasmid pOM163 (SEQ ID NO:25) is an example of a plasmid that can be used to install the strong constitutive P₁₅ promoter (SEQ ID NO:3) in a way that functionally couples the promoter to the carotenoid biosynthesis operon of C. glutamicum. Integration of the functional portion of pOM163 into a C. glutamicum strain by Campbelling in and Campbelling out also removes the native, MarR repressable, crt operon promoter and a portion of the marR gene, and installs a P₄₉₇ specR cassette that confers resistance to spectinomycin in C. glutamicum transformants.

Plasmid pOM163 was integrated into strain OM469 (see related US Patent Application BGI 180) to give strain OM609K. In shake flasks using molasses medium, as described in U.S. Provisional Patent Applications 60/714,042 and 60/700,699, incorporated by reference herein, OM469 and OM609K produced about 2.1 and 2.0 grams of methionine per liter, respectively, and an estimated 0.6 and 4.3 mg of decaprenoxanthin per gram dry weight of cells, respectively, after an extraction of the cell pellet with methanol:chloroform (1:1 by volume).

Plasmid pOM163 was integrated into strain OMI 82, which is a strain similar to OM134C described above, in that it is a derivative of M2014 (see related U.S. Provisional Patent Applications 60/714,042 and 60/700,699) that contains a disruption of the crtEb gene and therefore produces lycopene instead of decaprenoxanthin. The resulting strain is referred to as OM610K. In shake flasks using molasses medium (as described in U.S. Provisional Patent Applications 60/714,042 and 60/700,699), OM182 and OM610K produced about 1.1 and 0.9 grams of methionine per liter, respectively, and an estimated 0.3 and 5.7 mg of lycopene per gram dry weight of cells, respectively, after an extraction of the cell pellet with methanol:chloroform (1:1 by volume).

TABLE VI
Summary of vectors designed to integrate
at the C. glutamicum carotenoid operon

| | Promoter driving | Integration | |
|---|---|---|---|
| Vector | inserted gene | site | Color change |
| pOM245 (SEQ ID | P₄₉₇ | crtEb | yellow to pink |
| NoO.8) | | | |
| pOM246 (SEQ ID | P₁₅ | crtEb | yellow to pink |
| NO: o.14) | | | |
| pOM235F (SEQ ID | P₄₉₇ | marR | yellow to darker |
| NoO: .7) | | | yellow |
| pOM254 (SEQ ID | P₁₅ | marR | yellow to darker |
| NO: o.9) | | | yellow |
| pOM163 ((SEQ ID | P₄₉₇ | marR-crtE | yellow to darker |
| NOo: .25) | | junction | yellow or pink to |
| | | | darker pink |

The specification is most thoroughly understood in light of the teachings of the references cited within the specification which are hereby incorporated by reference. The embodiments within the specification provide an illustration of embodiments in this disclosure and should not be construed to limit its scope. The skilled artisan readily recognizes that many other embodiments are encompassed by this disclosure. All publications and patents cited and sequences identified by accession or database reference numbers in this disclosure are incorporated by reference in their entirety. To the extent the material incorporated by reference contradicts or is inconsistent with the present specification, the present specification will supercede any such material. The citation of any references herein is not an admission that such references are prior art to the present disclosure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A recombinant methionine producing microorganism, wherein said microorganism expresses a heterologous metI gene.

2. The microorganism of claim 1, wherein the metI gene is derived from the genus Bacillus.

3. The microorganism of claim 1 wherein the met gene is Bacillus subtilis metI.

4. The microorganism of claim 1, wherein the microorganism belongs to the genus Corynebacterium.

5. The microorganism of claim 4, wherein the microorganism is Corynebacterium glutamicum.

6. The microorganism of claim 1, wherein the microorganism comprises a deregulated MetI.

7. The microorganism of claim 6, wherein deregulation of metI is achieved by constitutive expression of a metI gene from a promoter and/or ribosome binding site that is not naturally associated with said metI gene.

8. A metI expression cassette, comprising metI operatively linked to a heterologous promoter and, optionally a ribosomal binding site.

9. The metI expression cassette of claim 8, wherein the promoter is P₁₅, P₄₉₇, P₁₂₈₄, P₃₁₁₉, λP_{R}, or λP_{L}.

10. A vector comprising the cassette of any one of claims 8-9.

11. A microorganism comprising the cassette of any one of claims 8-9.

12. A microorganism comprising the vector of claim 10.

13. A method for producing methionine, comprising culturing the microorganism of any one of claims 1 or 7 under conditions such that methionine is produced.

14. The method of claim 13, further comprising at least partially purifying the methionine.

15. A method for increasing methionine production capacity in a methionine-producing microorganism, comprising expressing a heterologous metI in said microorganism, such that methionine production capacity is increased.

16. A method for increasing methionine production capacity in a microorganism in which one or more methionine biosynthetic steps are subject to methionine feedback inhibition, comprising expressing a heterologous metI in said microorganism to alleviate methionine feedback inhibition, thereby increasing methionine production capacity.

17. The method of claim 16, wherein methionine production capacity is increased by at least 20% relative to a control microorganism.

18. The method of claim 16, wherein methionine production capacity is increased by at least 30% relative to a control microorganism.

19. The method of claim 16, wherein methionine production capacity is increased by at least 40% relative to a control microorganism.

20. The method of any one of claims 17-19, wherein the control microorganism does not comprise metI enzyme.

21. A DNA sequence that is capable of integrating at the Corynebacterium glutamicum crtEb locus (a crtEb integration cassette) comprising:
(a) a first DNA sequence;
(b) a second DNA sequence, and
(c) a third heterologous DNA sequence located between the first and the second DNA sequences,
wherein the first and the second DNA sequences are each homologous to a different portion of the C. glutamicum carotenoid biosynthetic operon, and wherein the third DNA sequence has an ability to disrupt a crtEb gene of a C. glutamicum strain by “Campbelling in” and “Campbelling out” derivatives of said strain.

22. The DNA sequence of claim 21, wherein the heterologous DNA sequence comprises an expression cassette comprising a metI gene.

23. A vector comprising the DNA sequence of any one of claims 21-22.

24. A microorganism comprising a vector of claim 23 or a portion of said vector.

25. A method for producing lycopene, comprising culturing a microorganism transformed with the integration cassette of claim 21 under conditions such that lycopene is produced.

26. A DNA sequence capable of integrating at the Corynebacterium glutamicum marR gene of the carotenoid biosynthetic locus comprising:
(a) a first DNA sequence;
(b) a second DNA sequence; and
(c) a third heterologous DNA sequence located between the first and the second DNA sequences, wherein the first and the second DNA sequences are each homologous to a different portion of the C. glutamicum carotenoid biosynthetic operon, and said DNA sequence has an ability to disrupt a marR gene of a C. glutamicum strain by “Campbelling in” and “Campbelling out” derivatives of said strain.

27. The DNA sequence of claim 26 wherein the heterologous DNA sequence comprises a metI gene.

28. A vector comprising the DNA sequence of any one of claims 26-27.

29. A microorganism comprising the vector of claim 28 or a portion of said vector.

30. A method for producing increased levels of a desired carotenoid, comprising culturing a microorganism transformed with the DNA sequence of claim 26 under conditions such that increased levels of the desired carotenoid are produced.

31. The method of claim 30, wherein the desired carotenoid is lycopene.

32. The method of claim 25 or 30, wherein the microorganism is a Corynebacterium.

33. A vector comprising an integration cassette chosen from a marR integration cassette and a crtEb integration cassette.

34. A microorganism comprising the vector of claim 33.

35. A method for producing at least two compounds in a fermentation process, in which the first compound that is produced is not a carotenoid, and the second compound that is produced comprises a carotenoid.

36. The method of claim 35, wherein the first compound is an amino acid.

37. The method of claim 36, wherein the amino acid is selected from the group consisting of methionine, lysine, glutamic acid, threonine, isoleucine, phenylalanine, tyrosine, tryptophan, alanine, cysteine, homoserine, homocysteine, and leucine.

38. The method of claim 35, wherein said first compound is a water soluble compound.

39. The method of claim 38, wherein said first compound is selected from the group consisting of lactic acid, 1,2-propane diol, 1,3-propane diol, ethanol, methanol, propanol, acetone, butanol, acetic acid, propionic acid, citric acid, itaconic acid, glucosamine, glycerol, sugar, vitamin, a therapeutic protein, a research protein, an industrial eprotein, a therapeutic enzyme, a research enzyme, an industrial enzyme, and a salt thereof.

40. The method of claim 35, wherein said the first compound is a gas.

41. The method of claim 40, wherein the gas is methane or hydrogen.

42. A method for producing a carotenoid compound which is a byproduct of an amino acid-producing fermentation process, comprising culturing a microorganism engineered to produce both increased levels of the amino acid and the carotenoid compound.

43. The method of claim 42, wherein culturing the microorganism comprises separating the culture into at least two components, one of which is enriched for the amino acid and one of which is enriched for the carotenoid.

44. The method of claim 42 or 43, wherein the amino acid is chosen from methionine, lysine, glutamic acid, threonine, isoleucine, phenylalanine, tyrosine, tryptophan, alanine, cysteine, homoserine, homocysteine and leucine.

45. The method of any of claims 42, wherein the carotenoid is chosen from decaprenoxanthin, lycopene, β-carotene, lutein, astaxanthin, canthaxanthin, bixin, and zeaxanthin.

46. A microorganism engineered to overproduce a first compound which is not a carotenoid, and a second compound which comprises a carotenoid compound.

47. The microorganism of claim 46, wherein said the first compound is an amino acid.

48. The microorganism of claim 46, wherein the first compound is an amino acid chosen from methionine, lysine, glutamic acid, threonine, isoleucine, phenylalanine, tyrosine, tryptophan, alanine, cysteine and leucine, and the second compound is chosen from decaprenoxanthin, lycopene, β-carotene, lutein, astaxanthin, canthaxanthin, bixin, and zeaxanthin.

49. The microorganism of claim 46, wherein said the first compound is chosen from methane, hydrogen, lactic acid, 1,2-propane diol, 1,3-propane diol, ethanol, methanol, propanol, acetone, butanol, acetic acid, propionic acid, citric acid, itaconic acid, glucosamine, glycerol, sugars, vitamins, therapeutic enzymes and proteins, research enzymes and proteins, industrial enzymes and proteins, and salts thereof and the second compound is chosen from decaprenoxanthin, lycopene, β-carotene, lutein, astaxanthin, canthaxanthin, bixin, and zeaxanthin.

50. A recombinant microorganism capable of producing a sulfur-containing fine chemical, comprising a heterologous metI gene.

51. A method for producing a sulfur-containing fine chemical comprising culturing the microorganism of claim 1 or claim 7 under conditions such that the sulfur containing fine-chemical is produced.

52. The DNA sequence of claim 26, wherein said third DNA sequence comprises a constitutive promoter that is functionally coupled to the first gene of said carotenoid biosynthetic operon, such that after integration into the genome of said C. glutamicum strain, said carotenoid biosynthetic operon is transcribed from said constitutive promoter.
